(19) United States
(12) Patent Application Publication
(10) Pub. No.: US 2024/0182910 A1
(43) Pub. Date: Jun. 6, 2024

(54) DRUG-REGULATABLE TRANSCRIPTION FACTORS

(71) Applicant: Senti Biosciences, Inc., South San Francisco, CA (US)

(72) Inventors: Michelle Elizabeth Hung, South San Francisco, CA (US); Russell Morrison Gordley, San Francisco, CA (US)

(21) Appl. No.: 18/489,693

(22) Filed: Oct. 18, 2023

Related U.S. Application Data
(63) Continuation of application No. PCT/US2022/025366, filed on Apr. 19, 2022.
(60) Provisional application No. 63/176,801, filed on Apr. 19, 2021.
(60) Provisional application No. 63/176,848, filed on Apr. 19, 2021.

(51) Int. Cl. C12N 15/63

(52) U.S. Cl. CPC C12N 15/635; C12N 2830/002

(57) ABSTRACT

Provided herein are synthetic inducible transcription factors that can be activated by a cognate ligand (e.g., caffeine, a cannabinoid, nicotine, rapamycin, or mifepristone) and isolated cells expressing the inducible transcription factors. Also provided are methods of modulating transcription of a gene of interest using an inducible transcription factor.

### CROSS REFERENCE TO RELATED APPLICATIONS

This application is a continuation of International Application No. PCT/US2022/025366, filed Apr. 19, 2022, which claims the benefit of and priority to U.S. Provisional Application No. 63/176,801, filed Apr. 19, 2021, and to U.S. Provisional Application No. 63/176,848, filed Apr. 19, 2021, each of which is hereby incorporated by reference in its entirety for all purposes.

### SEQUENCE LISTING

The instant application contains a Sequence Listing which has been submitted via patent Center and is hereby incorporated by reference in its entirety. Said XML copy, created on Nov. 6, 2023, is named STB-030WOC1.xml, and is 121,013 bytes in size.

### FIELD

The present application relates to synthetic inducible transcription factors that can be activated by a cognate ligand (e.g., caffeine, a cannabinoid, nicotine, rapamycin, or mifepristone) and isolated cells expressing the inducible transcription factors. Also provided are methods of modulating transcription of a gene of interest using an inducible transcription factor.

### BACKGROUND

Currently available cell and gene therapy products can lack expression control, causing safety concerns such as toxicity in subjects that receive such therapies. Thus, additional methods of expression control and regulation for these therapies are needed.

### SUMMARY

Provided herein, in one aspect, is an engineered cell comprising an inducible transcription factor (ITF), wherein the ITF comprises: a first monomer, wherein the first monomer comprises a DNA binding domain and a first ligand binding domain; and a second monomer, wherein the second monomer comprises a transcriptional effector domain and a second ligand binding domain, wherein each monomer is oligomerizable via a cognate ligand that binds to each ligand binding domain, wherein ligand-induced oligomerization forms the ITF, wherein the cognate ligand is selected from the group consisting of: caffeine, a cannabidiol (CBD), a phytocannabinoid, and nicotine, optionally wherein the DNA binding domain comprises a zinc finger (ZF) protein domain, optionally wherein each ligand binding domain comprises a single-domain antibody, wherein the single-domain antibody comprises a V_{H}H, and optionally wherein the ITF is capable of modulating transcription of a gene of interest operably linked to an ITF-responsive promoter.

In some embodiments,
a. the V_{H}H is an anti-caffeine V_{H}H (ac V_{H}H), optionally wherein the anti-caffeine V_{H}H (ac V_{H}H) comprises the amino acid sequence of SEQ ID NO: 52, and/or optionally wherein each of the first ligand binding domain and the second ligand binding domain comprises the ac V_{H}H; or
b. the cognate ligand is a cannabidiol or a phytocannabinoid, and the V_{H}H comprises an amino acid sequence selected from the group consisting of SEQ ID NO: 53, SEQ ID NO: 54, SEQ ID NO: 55, SEQ ID NO: 56, and SEQ ID NO: 57 (CA-14, DB-6, DB-11, DB-18, and DB-21, respectively), optionally wherein one of the first or the second ligand binding domain comprises a V_{H}H comprising the amino acid sequence of SEQ ID NO: 53 (CA-14) and the other of the first or the second binding domain comprises a V_{H}H comprising an amino acid sequence selected from the group consisting of: SEQ ID NO: 54, SEQ ID NO: 55, SEQ ID NO: 56, and SEQ ID NO: 57 (DB-6, DB-11, DB-18, and DB-21, respectively), optionally wherein one of the first or the second ligand binding domain comprises a V_{H}H comprising the amino acid sequence of SEQ ID NO: 53 (CA-14) and the other of the first or the second binding domain comprises a V_{H}H comprising an amino acid sequence selected from the group consisting of: SEQ ID NO: 54, SEQ ID NO: 55, SEQ ID NO: 56, and SEQ ID NO: 57 (DB-6, DB-11, DB-18, and DB-21, respectively), optionally wherein one of the first or the second ligand binding domain comprises a V_{H}H comprising the amino acid sequence of SEQ ID NO: 53 (CA-14) and the other of the first or the second binding domain comprises a V_{H}H comprising the amino acid sequence of SEQ ID NO: 57 (DB-21); or
c. each ligand binding domain comprises an anti-nicotine antibody heavy chain variable domain (anti-Nic VH) or an anti-nicotine antibody light chain variable domain (anti-Nic VL), optionally wherein the anti-Nic VH comprises the amino acid sequence of SEQ ID NO: 58 and the anti-Nic VL comprises the amino acid sequence of SEQ ID NO: 59, optionally wherein one of the first or the second ligand binding domain comprises the anti-Nic VH and the other of the first or the second ligand binding domain comprises the anti-Nic VL.

In another aspect, provided herein is an engineered cell comprising an inducible transcription factor (ITF), wherein the ITF comprises: a first monomer, wherein the first monomer comprises a DNA binding domain and a first ligand binding domain; and a second monomer, wherein the second monomer comprises a transcriptional effector domain and a second ligand binding domain, wherein each monomer is oligomerizable via binding of a cognate ligand that binds to each ligand binding domain, wherein ligand-induced oligomerization forms the ITF, wherein the DNA binding domain comprises a zinc finger (ZF) protein domain that is modular and comprises an array of six ZF motifs, wherein the cognate ligand is selected from the group consisting of: rapamycin, AP1903, AP20187, FK1012, derivatives thereof, and analogs thereof, optionally wherein the ITF is capable of modulating transcription of a gene of interest operably linked to an ITF-responsive promoter.

In some embodiments,
a. one of the first or the second ligand binding domain comprises an FKBP domain, and the other of the first or the second ligand binding domain comprises an FRB domain, optionally wherein the FKBP comprises the amino acid sequence of SEQ ID NO: 60, optionally wherein the FRB domain comprises the amino acid sequence of SEQ ID NO: 61; or
b. the first ligand binding domain comprises the FKBP domain and the second ligand binding domain comprises the FRB domain, optionally wherein the FKBP comprises the amino acid sequence of SEQ ID NO: 60, optionally wherein the FRB domain comprises the amino acid sequence of SEQ ID NO: 61; or
c. one of the first or the second ligand binding domain comprises the FRB domain, and the other of the first or the second ligand binding domain comprises the FKBP domain, optionally wherein the FKBP comprises the amino acid sequence of SEQ ID NO: 60, optionally wherein the FRB domain comprises the amino acid sequence of SEQ ID NO: 61; or
d. the first ligand binding domain comprises the FRB domain and the second ligand binding domain comprises the FKBP domain, optionally wherein the FKBP comprises the amino acid sequence of SEQ ID NO: 60, optionally wherein the FRB domain comprises the amino acid sequence of SEQ ID NO: 61.

In some embodiments,
a. the first monomer and/or the second monomer further comprises a nuclear localization signal (NLS),
   optionally wherein the NLS comprises an amino acid sequence selected from the group consisting of: SEQ ID NO: 2, SEQ ID NO: 3, and SEQ ID NO: 4; and/or
b. the second monomer further comprises a nuclear export signal (NES), optionally wherein the NES comprises an amino acid sequence selected from the group consisting of: SEQ ID NO: 5, SEQ ID NO: 6, SEQ ID NO: 7 SEQ ID NO: 8, SEQ ID NO: 9, SEQ ID NO: 10, SEQ ID NO: 11, SEQ ID NO: 12, SEQ ID NO: 13, and SEQ ID NO: 14.

In another aspect, provided herein is an engineered cell comprising an inducible transcription factor (ITF), wherein the ITF comprises a DNA binding domain, a transcriptional effector domain, and a ligand binding domain, wherein the ITF undergoes nuclear localization upon binding of the ligand binding domain to a cognate ligand, wherein when localized to a cell nucleus, the ITF is capable of modulating transcription of a gene of interest operably linked to an ITF-responsive promoter, wherein the cognate ligand is mifepristone or a derivative thereof, optionally wherein the ligand binding domain comprises a progesterone receptor domain and the progesterone receptor domain comprises the amino acid sequence of SEQ ID NO: 68, optionally wherein the DNA binding domain comprises a zinc finger (ZF) protein domain, optionally wherein the ITF further comprises a peptide linker between the DNA binding domain and the transcriptional effector domain, optionally wherein the DNA binding domain binds to the ITF-responsive promoter, and optionally wherein the cell further comprises an expression cassette comprising the ITF-responsive promoter operably linked to an exogenous polynucleotide sequence encoding the gene of interest, optionally wherein the ITF-responsive promoter comprises an ITF-binding domain sequence and a core promoter sequence, optionally wherein the core promoter sequence comprises a minimal promoter and the minimal promoter is selected from the group consisting of: minP, minCMV, YB_TATA, minTATA, and minTK, or wherein the core promoter sequence is derived from a constitutive promoter and the core promoter sequence is derived from a constitutive promoter selected from the group consisting of: CMV, EFS, SFFV, SV40, MND, PGK, UbC, EF1a, hCAGG, hACTb, heIF4A1, hGAPDH, hGRP78, hGRP94, hHSP70, hKINb, and hUBIb.

In some embodiments,
a. the ZF protein domain is modular in design and is composed of one or more zinc finger arrays (ZFA);
b. the ZF protein domain comprises an array of one to ten zinc finger motifs; and/or
c. the ZF protein domain comprises an array of six zinc finger motifs.

In some embodiments,
a. the transcriptional effector domain comprises a transcriptional activation domain, optionally wherein the transcriptional activation domain is selected from the group consisting of: a Herpes Simplex Virus Protein 16 (VP16) activation domain; an activation domain comprising four tandem copies of VP16; a VP64 activation domain; a p65 activation domain of NFκB; an Epstein-Barr virus R transactivator (Rta) activation domain; a tripartite activator comprising the VP64, the p65, and the Rta activation domains (VPR activation domain); and a histone acetyltransferase (HAT) core domain of the human E1A-associated protein p300 (p300 HAT core activation domain); or
b. the transcriptional effector domain comprises a transcriptional repressor domain, optionally wherein the transcriptional repressor domain is selected from the group consisting of: a Krüppel associated box (KRAB) repression domain; a truncated Krüppel associated box (KRAB) repression domain; a Histone Deacetylase 4 (HDAC4) repressor domain; a Scleraxis (SCX) HLH domain, an Inhibitor of DNA binding 1 (ID1) HLH domain, a HECT domain and RCC1-like domain-containing protein 2 (HERC2) Cyt-b5 domain, a Twist-related protein 1 (TWST1) HLH domain, an Homeobox protein Nkx-2.2 (NKX22) homeodomain, an Inhibitor of DNA binding 1 (ID3) HLH domain, and a Twist-related protein 2 (TWST2) HLH domain, and EED repressor domain; a Repressor Element Silencing Transcription Factor (REST) repression domain; a WRPW motif (SEQ ID NO: 76) of the hairy-related basic helix-loop-helix repressor proteins, the motif is known as a WRPW (SEQ ID NO: 76) repression domain; a DNA (cytosine-5)-methyltransferase 3B (DNMT3B) repression domain; and an HP1 alpha chromoshadow repression domain.

In another aspect, provided herein is an expression system comprising: a first expression cassette comprising a first promoter and an exogenous polynucleotide sequence encoding a first monomer, wherein the first monomer comprises a DNA binding domain and a first ligand binding domain; and a second expression cassette comprising a second promoter and an exogenous polynucleotide sequence encoding a second monomer, wherein the second monomer comprises a transcription effector domain and a second ligand binding domain, wherein each monomer is oligomerizable via a cognate ligand that binds to each ligand binding domain, wherein ligand-induced oligomerization of the first and the second monomer forms an inducible transcription factor (ITF), and wherein the cognate ligand is selected from the group consisting of: caffeine, a cannabidiol (CBD), a phytocannabinoid, and nicotine, optionally wherein the DNA binding domain comprises a zinc finger (ZF) protein domain, optionally wherein the ZF protein domain is modular in design and is composed of one or more zinc finger arrays (ZFA), optionally wherein the ZF protein domain comprises an array of one to ten zinc finger motifs, and optionally wherein the expression system further comprises a third expression cassette comprising an ITF-responsive promoter operably linked to a gene of interest, wherein the ITF-responsive promoter comprises a core promoter sequence and a sequence that binds to the DNA binding domain of the first engineered polypeptide.

In another aspect, provided herein is an expression system comprising: a first expression cassette comprising a first promoter and an exogenous polynucleotide sequence encoding a first monomer, wherein the first monomer comprises a DNA binding domain and a first ligand binding domain; and a second expression cassette comprising a second promoter and an exogenous polynucleotide sequence encoding a second monomer, wherein the second monomer comprises a transcriptional effector domain and a second ligand binding domain, wherein each monomer is oligomerizable via binding of a cognate ligand that binds to each ligand binding domain, wherein ligand-induced oligomerization of the first and the second monomer forms an inducible transcription factor (ITF), wherein the DNA binding domain comprises a zinc finger protein domain that is modular and comprises ten zinc finger arrays (ZFA), wherein the cognate ligand is selected from the group consisting of: rapamycin, AP1903, AP20187, FK1012, derivatives thereof, and analogs thereof, optionally wherein each ligand binding domain comprises an FKBP domain or an FRB domain, optionally wherein the first monomer and/or the second monomer further comprises a nuclear localization signal (NLS), optionally wherein the second monomer further comprises a nuclear export signal (NES), optionally wherein the ITF is capable of modulating transcription of a gene of interest operably linked to an ITF-responsive promoter, and optionally wherein the expression system further comprises a third expression cassette comprising an ITF-responsive promoter operably linked to a gene of interest, wherein the third promoter comprises a core promoter sequence and a sequence that binds to the DNA binding domain of the first engineered polypeptide.

In another aspect, provided herein is an expression system comprising a first expression cassette comprising a first promoter and an exogenous polynucleotide sequence encoding an inducible transcription factor (ITF), wherein the ITF comprises a DNA binding domain, a transcriptional effector domain, and a ligand binding domain, wherein the ITF undergoes nuclear localization upon binding of the ligand binding domain to a cognate ligand, wherein when localized to a cell nucleus, the ITF is capable of modulating transcription of a gene of interest operably linked to an ITF-responsive promoter, wherein the cognate ligand is mifepristone or a derivative thereof, optionally wherein the ligand binding domain comprises a progesterone receptor domain, optionally wherein the DNA binding domain comprises a zinc finger (ZF) protein domain, optionally wherein the ZF protein domain is modular in design and is composed of one or more zinc finger arrays (ZFA), optionally wherein the ZF protein domain comprises one to ten zinc finger motifs, optionally wherein the ITF further comprises a linker localized between the DNA binding domain and the transcriptional effector domain, and optionally wherein the expression system further comprises an expression cassette comprising the ITF-responsive promoter operably linked to an exogenous polynucleotide sequence encoding the gene of interest.

In another aspect, provided herein is an engineered cell comprising the expression system of any of the above aspects or embodiments.

In certain embodiments,
a. the cell comprises a human cell; and/or
b. the cell comprises a stem cell; and/or
c. the cell comprises an immune cell; and/or
d. the cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an erythrocyte, a platelet cell, a human embryonic stem cell (ESC), an ESC-derived cell, a pluripotent stem cell, a mesenchymal stromal cell (MSC), an induced pluripotent stem cell (iPSC), and an iPSC-derived cell.

In another aspect, provided herein is a genetic switch for modulating transcription of a gene of interest, comprising:
a. the engineered cell of any one of the above aspects or embodiments and a cognate ligand selected from the group consisting of: caffeine, a cannabidiol (CBD), a phytocannabinoid, and nicotine, to induce formation of the inducible transcription factor (ITF); or
b. the engineered cell of any one of the above aspects or embodiments and a cognate ligand selected from the group consisting of: rapamycin, AP1903, AP20187, FK1012, derivatives thereof, and analogs thereof; or
c. the engineered cell of any one of the above aspects or embodiments and mifepristone, or a derivative thereof.

In another aspect, provided herein is a method of modulating transcription of a gene of interest, comprising:
a. contacting the engineered cell of any one of the above aspects or embodiments with a cognate ligand selected from the group consisting of: caffeine, a cannabidiol (CBD), a phytocannabinoid, and nicotine, to induce formation of the inducible transcription factor (ITF), optionally further comprising culturing the engineered cell for a time period sufficient to allow the ITF to modulate transcription of a gene operably linked to an ITF-responsive promoter, optionally wherein the contacting is performed in a human or animal, and optionally wherein contacting the transformed cell with the cognate ligand comprises administering a pharmacological dose of the cognate ligand to the human or animal; or
b. contacting the engineered cell of any one of the above aspects or embodiments with a cognate ligand selected from the group consisting of: rapamycin, AP1903, AP20187, FK1012, derivatives thereof, and analogs thereof to induce formation of the inducible transcription factor (ITF), optionally further comprising culturing the engineered cell for a time period sufficient to allow the ITF to modulate transcription of a gene operably linked to an ITF-responsive promoter, optionally wherein the engineered cell is in a human or animal, and wherein contacting the engineered cell with the cognate ligand comprises administering a pharmacological dose of the rapamycin to the human or animal, optionally wherein the transcription effector domain of the second monomer comprises a transcriptional activation domain and modulating transcription comprises activating transcription of the gene of interest, or wherein the transcription effector domain of the second monomer comprises a transcriptional repressor domain and modulating transcription comprises repressing transcription of the gene of interest; or
c. contacting the engineered cell of any one of the above aspects or embodiments with mifepristone, or a derivative thereof, to induce nuclear localization of the inducible transcription factor (ITF), optionally further comprising culturing the engineered cell for a time period sufficient to allow the ITF to modulate transcription of a gene operably linked to an ITF-responsive promoter, optionally wherein the engineered cell is in a human or animal, and wherein contacting the engineered cell with the mifepristone comprises administering a pharmacological dose of the mifepristone to the human or animal, optionally wherein the transcriptional effector domain comprises a transcriptional activation domain and modulating transcription comprises activating transcription of the gene of interest, or wherein the transcriptional effector domain comprises a transcriptional repressor domain and modulating transcription comprises repressing transcription of the gene of interest.

Also provided herein are inducible transcription factors that can be activated by a cognate ligand selected from caffeine, a cannabinoid, nicotine, and rapamycin.

The inducible transcription factors described herein are activatable by ligands that can be used in vivo, such as for controlling gene expression in a therapeutic cell.

In some aspects, the present disclosure provides an engineered cell comprising an inducible transcription factor (ITF), wherein the ITF comprises: (i) a first monomer, wherein the first monomer comprises a DNA binding domain and a first ligand binding domain; and (II) a second monomer, wherein the second monomer comprises a transcriptional effector domain and a second ligand binding domain, wherein each monomer is oligomerizable via a cognate ligand that binds to each ligand binding domain, wherein ligand-induced oligomerization forms the ITF, and wherein the cognate ligand is selected from the group consisting of: caffeine, a cannabidiol (CBD), a phytocannabinoid, and nicotine.

In some aspects, the DNA binding domain comprises a zinc finger (ZF) protein domain. In some aspects, the ZF protein domain is modular in design and is composed of one or more zinc finger arrays (ZFA). In some aspects, the ZF protein domain comprises an array of one to ten ZF motifs. In some aspects, the ZF protein domain comprises six ZF motifs.

In some aspects, the first ligand binding domain and the second ligand binding domain bind a distinct epitope of the cognate ligand.

In some aspects, each ligand binding domain comprises a single-domain antibody.

In some aspects, the single-domain antibody comprises a V_{H}H. In some aspects, the V_{H}H is an anti-caffeine V_{H}H (ac V_{H}H). In some aspects, the anti-caffeine V_{H}H (acV_{H}H) comprises the amino acid sequence of SEQ ID NO: 52. In some aspects, each of the first ligand binding domain and the second ligand binding domain comprises the ac V_{H}H.

In some aspects, the cognate ligand is a cannabidiol or a phytocannabinoid, and the V_{H}H comprises an amino acid sequence selected from the group consisting of SEQ ID NO: 53-57 (CA14, DB6, DB11, DB18, and DB21).

In some aspects, one of the first or the second ligand binding domain comprises a V_{H}H comprising the amino acid sequence of SEQ ID NO: 53 (CA-14) and the other of the first or the second binding domain comprises a V_{H}H comprising an amino acid sequence selected from the group consisting of: 54-57 (DB6, DB11, DB18, and DB21).

In some aspects, one of the first or the second ligand binding domain comprises a V_{H}H comprising the amino acid sequence of SEQ ID NO: 53 (CA-14) and the other of the first or the second binding domain comprises a V_{H}H comprising the amino acid sequence of SEQ ID NO: 54 (DB-21).

In some aspects, each ligand binding domain comprises an anti-nicotine antibody heavy chain variable domain (anti-Nic VH) or an anti-nicotine antibody light chain variable domain (anti-Nic VL).

In some aspects, the anti-Nic VH comprises the amino acid sequence of SEQ ID NO: 58.

In some aspects, the anti-Nic VL comprises the amino acid sequence of SEQ ID NO: 59.

In some aspects, one of the first or the second ligand binding domain comprises the anti-Nic VH and the other of the first or the second ligand binding domain comprises the anti-Nic VL.

In some aspects, the present disclosure provides an engineered cell comprising an inducible transcription factor (ITF), wherein the ITF comprises: (i) a first monomer, wherein the first monomer comprises a DNA binding domain and a first ligand binding domain; and (ii) a second monomer, wherein the second monomer comprises a transcriptional effector domain and a second ligand binding domain, wherein each monomer is oligomerizable via binding of a cognate ligand that binds to each ligand binding domain, wherein ligand-induced oligomerization forms the ITF, wherein the DNA binding domain comprises a zinc finger (ZF) protein domain that is modular and comprises an array of six ZF motifs, and wherein the cognate ligand is selected from the group consisting of: rapamycin, AP1903, AP20187, FK1012, derivatives thereof, or analogs thereof.

In some aspects, one of the first or the second ligand binding domain comprises an FKBP domain, and the other of the first or the second ligand binding domain comprises an FRB domain.

In some aspects, the FKBP comprises the amino acid sequence of SEQ ID NO: 60.

In some aspects, the FRB domain comprises the amino acid sequence of SEQ ID NO: 61.

In some aspects, the first ligand binding domain comprises the FKBP domain and the second ligand binding domain comprises the FRB domain.

In some aspects, the one of the first or the second ligand binding domain comprises the FRB domain, and the other of the first or the second ligand binding domain comprises the FKBP domain.

In some aspects, the first monomer and/or the second monomer further comprises a nuclear localization signal (NLS).

In some aspects, the NLS comprises an amino acid sequence selected from the group consisting of: SEQ ID NO: 2, SEQ ID NO: 3, and SEQ ID NO: 4.

In some aspects, the second monomer further comprises a nuclear export signal (NES).

In some aspects, the NES comprises an amino acid sequence selected from the group consisting of: SEQ ID NO: 5, SEQ ID NO: 6, SEQ ID NO: 7 SEQ ID NO: 8, SEQ ID NO: 9, SEQ ID NO: 10, SEQ ID NO: 11, SEQ ID NO: 12, SEQ ID NO: 13, and SEQ ID NO: 14.

In some aspects, the ITF is capable of modulating transcription of a gene of interest operably linked to an ITF-responsive promoter.

In some aspects, the present disclosure provides an expression system comprising: (i) a first expression cassette comprising a first promoter and an exogenous polynucleotide sequence encoding a first monomer, wherein the first monomer comprises a DNA binding domain and a first ligand binding domain; and (ii) a second expression cassette comprising a second promoter and an exogenous polynucleotide sequence encoding a second monomer, wherein the second monomer comprises a transcription effector domain and a second ligand binding domain, wherein each monomer is oligomerizable via a cognate ligand that binds to each ligand binding domain, wherein ligand-induced oligomerization of the first and the second monomer forms an inducible transcription factor (ITF), and wherein the cognate ligand is selected from the group consisting of: caffeine, a cannabidiol (CBD), a phytocannabinoid, and nicotine.

In some aspects, the DNA binding domain comprises a zinc finger (ZF) protein domain.

In some aspects, the ZF protein domain is modular in design and is composed of one or more zinc finger arrays (ZFA).

In some aspects, the ZF protein domain comprises an array of one to ten zinc finger motifs.

In some aspects, the ZF protein domain comprises an array of six zinc finger motifs.

In some aspects, each ligand binding domain comprises a single-domain antibody.

In some aspects, the single-domain antibody comprises a V_{H}H.

In some aspects, the V_{H}H is an anti-caffeine V_{H}H (acV_{H}H).

In some aspects, the anti-caffeine V_{H}H (acV_{H}H) comprises the amino acid sequence of SEQ ID NO: 52. In some aspects, each of the first ligand binding domain and the second ligand binding domain comprises the ac V_{H}H.

In some aspects, the first monomer and second monomer are capable of forming a homodimer upon binding to caffeine.

In some aspects, the cognate ligand is a cannabidiol or a phytocannabinoid, and the V_{H}H comprises an amino acid sequence selected from the group consisting of SEQ ID NO: 53-57 (CA14, DB6, DB11, DB18, and DB21).

In some aspects, the first ligand binding domain comprises a V_{H}H comprising the amino acid sequence of SEQ ID NO: 53 (CA-14) and the second binding domain comprises a V_{H}H comprising an amino acid sequence selected from the group consisting of: 54-57 (DB6, DB11, DB18, and DB21).

In some aspects, the first ligand binding domain comprises a V_{H}H comprising the amino acid sequence of SEQ ID NO: 53 (CA-14) and the second binding domain comprises a V_{H}H comprising the amino acid sequence of SEQ ID NO: 54-57 (DB-21).

In some aspects, the first monomer and second monomer are capable of forming a heterodimer upon binding to a cannabidiol or a phytocannabinoid.

In some aspects, each ligand binding domain comprises an anti-nicotine antibody heavy chain variable domain (anti-Nic VH) or an anti-nicotine antibody light chain variable domain (anti-Nic VL).

In some aspects, the anti-Nic VH comprises the amino acid sequence of SEQ ID NO: 58.

In some aspects, the anti-Nic VL comprises the amino acid sequence of SEQ ID NO: 59.

In some aspects, the first ligand binding domain comprises the anti-Nic VH and the second binding domain comprises the anti-Nic VL.

In some aspects, the first ligand binding domain comprises the anti-Nic VL and the second ligand binding domain comprises the anti-Nic VH.

In some aspects, the first monomer and second monomer are capable of forming a heterodimer upon binding to nicotine.

In some aspects, the expression system further comprises a third expression cassette comprising an ITF-responsive promoter operably linked to a gene of interest, wherein the ITF-responsive promoter comprises a core promoter sequence and a sequence that binds to the DNA binding domain of the first engineered polypeptide.

In some aspects, the core promoter sequence comprises a minimal promoter. In some aspects, the minimal promoter is selected from the group consisting of: minP, minCMV, YB_TATA, minTATA, and minTK.

In some aspects, the core promoter sequence is derived from a constitutive promoter. In some aspects, the core promoter sequence is derived from a constitutive promoter selected from the group consisting of: CMV, EFS, SFFV, SV40, MND, PGK, UbC, EF1a, hCAGG, hACTb, heIF4A1, hGAPDH, hGRP78, hGRP94, hHSP70, hKINb, and hUBIb.

In some aspects, the DNA binding domain comprises a zinc finger (ZF) protein domain, and the sequence that binds to the DNA binding domain comprises one or more ZF binding sites.

In some aspects, the present disclosure provides an expression system comprising: (i) a first expression cassette comprising a first promoter and an exogenous polynucleotide sequence encoding a first monomer, wherein the first monomer comprises a DNA binding domain and a first ligand binding domain; and (ii) a second expression cassette comprising a second promoter and an exogenous polynucleotide sequence encoding a second monomer, wherein the second monomer comprises a transcriptional effector domain and a second ligand binding domain, wherein each monomer is oligomerizable via binding of a cognate ligand that binds to each ligand binding domain, wherein ligand-induced oligomerization of the first and the second monomer forms an inducible transcription factor (ITF), wherein the DNA binding domain comprises a zinc finger protein domain that is modular and comprises six zinc finger motifs, and wherein the cognate ligand is selected from the group consisting of: rapamycin, AP1903, AP20187, FK1012, derivatives thereof, or analogs thereof.

In some aspects, each ligand binding domain comprises an FKBP domain or an FRB domain. In some aspects, the FKBP comprises the amino acid sequence of SEQ ID NO: 60.

In some aspects, the FRB domain comprises the amino acid sequence of SEQ ID NO: 61. In some aspects, the first ligand binding domain comprises the FKBP domain and the second ligand binding domain comprises the FRB domain. In some aspects, the first ligand binding domain comprises the FRB domain and the second ligand binding domain comprises the FKBP domain.

In some aspects, the first monomer and the second monomer are capable of forming a heterodimer upon binding to a ligand selected from the group consisting of: rapamycin, AP1903, AP20187, FK1012, derivatives thereof, or analogs thereof.

In some aspects, the first monomer and/or the second monomer further comprises a nuclear localization signal (NLS). In some aspects, the NLS comprises an amino acid sequence selected from the group consisting of: SEQ ID NO: 2, SEQ ID NO: 3, and SEQ ID NO: 4.

In some aspects, the second monomer further comprises a nuclear export signal (NES). In some aspects, the NES comprises an amino acid sequence selected from the group consisting of: SEQ ID NO: 5, SEQ ID NO: 6, SEQ ID NO: 7 SEQ ID NO: 8, SEQ ID NO: 9, SEQ ID NO: 10, SEQ ID NO: 11, SEQ ID NO: 12, SEQ ID NO: 13, and SEQ ID NO: 14.

In some aspects, the ITF is capable of modulating transcription of a gene of interest operably linked to an ITF-responsive promoter.

In some aspects, the first and/or second promoter is a constitutive promoter or a regulatable promoter.

In some aspects, the first and/or the second promoter is a synthetic promoter.

In some aspects, the first promoter, the second promoter, or both the first and second promoters is a constitutive promoter selected from the group consisting of: CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, and hUBIb.

In some aspects, the expression system further comprises a third expression cassette comprising an ITF-responsive promoter operably linked to a gene of interest, wherein the third promoter comprises a core promoter sequence and a sequence that binds to the DNA binding domain of the first engineered polypeptide. In some aspects, the core promoter sequence comprises a minimal promoter.

In some aspects, the transcriptional effector domain comprises a transcriptional activation domain. In some aspects, the transcriptional activation domain is selected from the group consisting of: a Herpes Simplex Virus Protein 16 (VP16) activation domain; an activation domain comprising four tandem copies of VP16; a VP64 activation domain; a p65 activation domain of NFκB; an Epstein-Barr virus R transactivator (Rta) activation domain; a tripartite activator comprising the VP64, the p65, and the Rta activation domains (VPR activation domain); and a histone acetyltransferase (HAT) core domain of the human E1A-associated protein p300 (p300 HAT core activation domain).

In some aspects, the transcriptional effector domain comprises a transcriptional repressor domain. In some aspects, the transcriptional repressor domain is selected from the group consisting of: a Krüppel associated box (KRAB) repression domain; a Histone Deacetylase 4 (HDAC4) repressor domain; a Scleraxis (SCX) HLH domain, an Inhibitor of DNA binding 1 (ID1) HLH domain, a HECT domain and RCC1-like domain-containing protein 2 (HERC2) Cyt-b5 domain, a Twist-related protein 1 (TWST1) HLH domain, an Homeobox protein Nkx-2.2 (NKX22) homeodomain, an Inhibitor of DNA binding 1 (ID3) HLH domain, and a Twist-related protein 2 (TWST2) HLH domain, an EED repressor domain; a Repressor Element Silencing Transcription Factor (REST) repression domain; a WRPW motif (SEQ ID NO: 76) of the hairy-related basic helix-loop-helix repressor proteins, the motif is known as a WRPW (SEQ ID NO: 76) repression domain; a DNA (cytosine-5)-methyltransferase 3B (DNMT3B) repression domain; and an HP1 alpha chromoshadow repression domain.

In some aspects, the DNA binding domain binds to the ITF-responsive promoter.

In some aspects, the first promoter is a constitutive promoter, a regulatable promoter, or a synthetic promoter.

In some aspects, the first promoter is a constitutive promoter selected from the group consisting of: CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, and hUBIb.

In some aspects, the expression system further comprises an expression cassette comprising the ITF-responsive promoter operably linked to an exogenous polynucleotide sequence encoding the gene of interest.

In some aspects, the ITF-responsive promoter comprises an ITF-binding domain sequence and a promoter sequence.

In some aspects, the promoter sequence is derived from a promoter selected from the group consisting of: minP, NFκB response element, CREB response element, NFAT response element, SRF response element 1, SRF response element 2, AP1 response element, TCF-LEF response element promoter fusion, Hypoxia responsive element, SMAD binding element, STAT3 binding site, minCMV, YB_TATA, minTATA, minTK, inducer molecule-responsive promoters, and tandem repeats thereof.

In some aspects, the ITF-responsive promoter comprises a minimal promoter.

In some aspects, the present disclosure provides an engineered cell comprising an expression system as described herein.

In some aspects, the engineered cell is a human cell.

In some aspects, the engineered cell is a stem cell.

In some aspects, the engineered cell is an immune cell.

In some aspects, the engineered cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an erythrocyte, a platelet cell, a human embryonic stem cell (ESC), an ESC-derived cell, a pluripotent stem cell, a mesenchymal stromal cell (MSC), an induced pluripotent stem cell (iPSC), and an iPSC-derived cell.

In some aspects, the present disclosure provides a genetic switch for modulating transcription of a gene of interest, comprising: an engineered cell comprising a caffeine-ITF; and an effective amount of caffeine.

In some aspects, the present disclosure provides a genetic switch for modulating transcription of a gene of interest, comprising: the engineered cell comprising a phytocannabinoid-ITF; and an effective amount of cannabidiol (CBD) or a phytocannabinoid.

In some aspects, the present disclosure provides a genetic switch for modulating transcription of a gene of interest, comprising: an engineered cell comprising a nicotine-ITF; and an effective amount of nicotine.

In some aspects, the present disclosure provides a genetic switch for modulating transcription of a gene of interest, comprising: the engineered cell a rapamycin-ITF, and effective amount of rapamycin.

In some aspects, the present disclosure provides a method of modulating transcription of a gene of interest, comprising: contacting an engineered cell comprising an ITF inducible by caffeine, a phytocannabinoid or nicotine with a cognate ligand selected from the group consisting of: caffeine, a cannabidiol (CBD), a phytocannabinoid, and nicotine, to induce formation of the inducible transcription factor (ITF).

In some aspects, the method further comprises culturing the engineered cell for a time period sufficient to allow the ITF to modulate transcription of a gene operably linked to an ITF-responsive promoter.

In some aspects, the contacting is performed in a human or animal.

In some aspects, contacting the transformed cell with the cognate ligand comprises administering a pharmacological dose of the cognate ligand to the human or animal.

In some aspects, the cognate ligand comprises caffeine.

In some aspects, the cognate ligand is cannabidiol (CBD)

In some aspects, the cognate ligand is nicotine.

In some aspects, the present disclosure provides a method of modulating transcription of a gene of interest, comprising: contacting an engineered cell comprising a rapamycin-ITF with rapamycin to induce formation of the inducible transcription factor (ITF).

In some aspects, the method further comprises culturing the engineered cell for a time period sufficient to allow the ITF to modulate transcription of a gene operably linked to an ITF-responsive promoter.

In some aspects, the engineered cell is in a human or animal, and wherein contacting the engineered cell with the cognate ligand comprises administering a pharmacological dose of the rapamycin to the human or animal.

In some aspects, the transcription effector domain of the second monomer comprises a transcriptional activation domain, and wherein modulating transcription comprises activating transcription of the gene of interest.

In some aspects, the transcription effector domain of the second monomer comprises a transcriptional repressor domain, wherein modulating transcription comprises repressing transcription of the gene of interest.

Also provided herein are inducible transcription factors that can be activated by mifepristone.

The inducible transcription factors described herein can be used in vivo, such as for controlling gene expression in therapeutic cell.

In some aspects, the present disclosure provides an engineered cell comprising an inducible transcription factor (ITF), wherein the ITF comprises a DNA binding domain, a transcriptional effector domain, and a ligand binding domain, wherein the ITF undergoes nuclear localization upon binding of the ligand binding domain to a cognate ligand, wherein when localized to a cell nucleus, the ITF is capable of modulating transcription of a gene of interest operably linked to an ITF-responsive promoter, and wherein the cognate ligand is mifepristone or a derivative thereof. In some aspects, the ligand binding domain comprises a progesterone receptor domain. In some aspects, the progesterone receptor domain comprises the amino acid sequence of SEQ ID NO: 68.

In some aspects, the DNA binding domain comprises a zinc finger (ZF) protein domain. In some aspects, the ZF protein domain is modular in design and is composed of one or more zinc finger arrays (ZFA). In some aspects, the ZF protein domain comprises one to ten zinc finger motifs. In some aspects, the ZF protein domain comprises six zinc finger motifs.

In some aspects, the ITF further comprises a peptide linker between the DNA binding domain and the transcriptional effector domain.

In some aspects, the DNA binding domain binds to the ITF-responsive promoter. In some aspects, the ITF-responsive promoter comprises an ITF-binding domain sequence and a core promoter sequence. In some aspects, the core promoter sequence comprises a minimal promoter. In some aspects, the minimal promoter is selected from the group consisting of: minP, minCMV, YB_TATA, minTATA, and minTK. In some aspects, the core promoter sequence is derived from a constitutive promoter. In some aspects, the core promoter sequence is derived from a constitutive promoter selected from the group consisting of: CMV, EFS, SFFV, SV40, MND, PGK, UbC, EF1a, hCAGG, hACTb, heIF4A1, hGAPDH, hGRP78, hGRP94, hHSP70, hKINb, and hUBIb.

In some aspects, the cell further comprises an expression cassette comprising the ITF-responsive promoter operably linked to an exogenous polynucleotide sequence encoding the gene of interest.

In some aspects, the transcriptional effector domain comprises a transcriptional activation domain. In some aspects, the transcriptional activation domain is selected from the group consisting of: a Herpes Simplex Virus Protein 16 (VP16) activation domain; an activation domain comprising four tandem copies of VP16; a VP64 activation domain; a p65 activation domain of NFκB; an Epstein-Barr virus R transactivator (Rta) activation domain; a tripartite activator comprising the VP64, the p65, and the Rta activation domains (VPR activation domain); and a histone acetyltransferase (HAT) core domain of the human E1A-associated protein p300 (p300 HAT core activation domain).

In some aspects, the transcriptional effector domain comprises a transcriptional repressor domain. In some aspects, the transcriptional repressor domain is selected from the group consisting of: a Krüppel associated box (KRAB) repression domain; a truncated Krüppel associated box (KRAB) repression domain; a Histone Deacetylase 4 (HDAC4) repressor domain; a Scleraxis (SCX) HLH domain, an Inhibitor of DNA binding 1 (ID1) HLH domain, a HECT domain and RCC1-like domain-containing protein 2 (HERC2) Cyt-b5 domain, a Twist-related protein 1 (TWST1) HLH domain, an Homeobox protein Nkx-2.2 (NKX22) homeodomain, an Inhibitor of DNA binding 1 (ID3) HLH domain, and a Twist-related protein 2 (TWST2) HLH domain, and EED repressor domain; a Repressor Element Silencing Transcription Factor (REST) repression domain; a WRPW motif (SEQ ID NO: 76) of the hairy-related basic helix-loop-helix repressor proteins, the motif is known as a WRPW (SEQ ID NO: 76) repression domain; a DNA (cytosine-5)-methyltransferase 3B (DNMT3B) repression domain; and an HP1 alpha chromoshadow repression domain.

In some aspects, the present disclosure provides an expression system comprising a first expression cassette comprising a first promoter and an exogenous polynucleotide sequence encoding an inducible transcription factor (ITF), wherein the ITF comprises a DNA binding domain, a transcriptional effector domain, and a ligand binding domain, wherein the ITF undergoes nuclear localization upon binding of the ligand binding domain to a cognate ligand, wherein when localized to a cell nucleus, the ITF is capable of modulating transcription of a gene of interest operably linked to an ITF-responsive promoter, and wherein the cognate ligand is mifepristone or a derivative thereof. In some aspects, the DNA binding domain binds to the ITF-responsive promoter. In some aspects, the first promoter is a constitutive promoter, a regulatable promoter, or a synthetic promoter. In some aspects, the first promoter is a constitutive promoter selected from the group consisting of: CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, and hUBIb.

In some aspects, the expression system further comprises an expression cassette comprising the ITF-responsive promoter operably linked to an exogenous polynucleotide sequence encoding the gene of interest. In some aspects, the ITF-responsive promoter comprises an ITF-binding domain sequence and a core promoter sequence. In some aspects, the core promoter sequence comprises a minimal promoter. In some aspects, the minimal promoter is selected from the group consisting of: minP, minCMV, YB_TATA, minTATA, and minTK. In some aspects, the core promoter sequence is derived from a constitutive promoter. In some aspects, the core promoter sequence is derived from a constitutive promoter selected from the group consisting of: CMV, EFS, SFFV, SV40, MND, PGK, UbC, EF1a, hCAGG, hACTb, heIF4A1, hGAPDH, hGRP78, hGRP94, hHSP70, hKINb, and hUBIb.

In some aspects, the present disclosure provides an engineered cell comprising any of the expression systems as described herein.

In some aspects, the engineered cell comprises a human cell. In some aspects, the engineered cell comprises a stem cell. In some aspects, the engineered cell comprises an immune cell. In some aspects, the cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an erythrocyte, a platelet cell, a human embryonic stem cell (ESC), an ESC-derived cell, a pluripotent stem cell, a mesenchymal stromal cell (MSC), an induced pluripotent stem cell (iPSC), and an iPSC-derived cell.

In some aspects, the present disclosure provides a genetic switch for modulating transcription of a gene of interest, comprising: any engineered cell as described herein; and an effective amount of mifepristone.

In some aspects, the present disclosure provides a method of modulating transcription of a polypeptide of interest, comprising contacting any engineered cell as described herein with mifepristone to induce nuclear localization of the inducible transcription factor (ITF). In some aspects, the method further comprises culturing the engineered cell for a time period sufficient to allow the ITF to modulate transcription of a gene operably linked to an ITF-responsive promoter. In some aspects, the engineered cell is in a human or animal, and wherein contacting the engineered cell with the mifepristone comprises administering a pharmacological dose of the mifepristone to the human or animal. In some aspects, the transcriptional effector domain comprises a transcriptional activation domain, and wherein modulating transcription comprises activating transcription of the gene of interest. In some aspects, the transcriptional effector domain comprises a transcriptional repressor domain, and wherein modulating transcription comprises repressing transcription of the gene of interest.

### BRIEF DESCRIPTION OF THE SEVERAL VIEWS OF THE DRAWINGS

These and other features, aspects, and advantages of the present disclosure will become better understood with regard to the following description, and accompanying drawings.

FIG. 1 shows the fold change in reporter expression following treatment with various concentrations of cognate ligand, for cells expressing inducible transcription factors.

FIG. 2 shows the percentage of cells positive for reporter expression following treatment with various concentrations of cognate ligand, for cells expressing caffeine-, nicotine-, or cannabinoid-inducible transcription factors with the first and the second monomers each including a nuclear localization signal.

FIG. 3 shows the mean fluorescent intensity of reporter expression following treatment with various concentrations of cognate ligand, for cells expressing caffeine-, nicotine-, or cannabinoid-inducible transcription factors with the first and the second monomers each including a nuclear localization signal.

FIG. 4 shows the fold change in reporter expression following treatment with various concentrations of mifepristone, for cells expressing a mifepristone-inducible transcription factor.

### DETAILED DESCRIPTION

Terms used in the claims and specification are defined as set forth below unless otherwise specified.

The term “in vivo” refers to processes that occur in a living organism.

The term “mammal” as used herein includes both humans and non-humans and include but is not limited to humans, non-human primates, canines, felines, murines, bovines, equines, and porcines.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

The term “sufficient amount” means an amount sufficient to produce a desired effect, e.g., an amount sufficient to inhibit transcriptional repression in a cell.

It must be noted that, as used in the specification and the appended claims, the singular forms “a,” “an” and “the” include plural referents unless the context clearly dictates otherwise.

### Inducible Transcription Factors

Provided herein are inducible transcription factors (ITF) with activity that can be induced by a cognate ligand. Also provided herein are inducible transcription factors with activity that can be induced by mifepristone.

In some embodiments, the inducible transcription factor includes a first monomer, the first monomer including a DNA binding domain and a first ligand binding domain; and a second monomer, the second monomer including a transcriptional effector domain and a second ligand binding domain. Upon binding of a cognate ligand to the first monomer and the second monomer, the first and second monomers oligomerize (e.g., dimerize), and ligand-induced oligomerization of the first and second monomer forms the ITF. In some embodiments, the first monomer is a first antibody domain and the second monomer is a second antibody domain, and the first antibody domain and the second antibody domain are oligomerizable. In some embodiments, the inducible transcription factor is a polypeptide including a DNA binding domain, a transcriptional effector domain, and a ligand binding domain. Upon binding of a cognate ligand to the ligand binding domain, ITF undergoes nuclear localization, where the ITF is capable of modulating transcription of a gene of interest operably linked to an ITF-responsive promoter.

“Ligand binding domain” refers to a polypeptide domain that specifically binds to a cognate ligand. As used herein, the terms “specifically binds,” and “specifically recognizes” are analogous and refer binding of a polypeptide molecule (e.g., a ligand binding domain) to a ligand (e.g., caffeine, a cannabinoid, nicotine, rapamycin, or mifepristone) as such binding is understood by one skilled in the art. For example, a ligand binding domain that specifically binds to a cognate ligand may bind to other ligands with lower affinity as determined by, e.g., immunoassays, BIAcore®, KinExA 3000 instrument (Sapidyne Instruments, Boise, Id.), or other assays known in the art. In a specific embodiment, a ligand binding domain that specifically binds to a cognate ligand is capable of binding to the cognate ligand a KA that is at least 2-fold, 5-fold, 10-fold, 20-fold, 25-fold, 30-fold, or greater than the KA when the ligand binding domain bind non-specifically to another ligand.

“DNA binding domain” as used herein refers to a protein domain that contains at least one structural motif that recognizes double- or single-stranded DNA. A DNA binding domain can recognize a specific DNA sequence (a recognition sequence) or have a general affinity to DNA. An example of a DNA binding domain that is capable of specific sequence recognition is a zinc finger (ZF) protein domain.

“Zinc finger (ZF) protein domain” as used herein refers to a protein at least one motif that contains multiple finger-like protrusions that make tandem contacts with DNA. “Zinc finger array (ZFA)” as used herein refers to multiple zinc finger protein motifs that are linked together. Each zinc finger motif binds to a different nucleic acid motif. This results in a ZFA with specificity to any desired nucleic acid sequence. The ZF motifs can be directly adjacent to each other, or separated by a flexible linker sequence. In some embodiments, a ZFA is an array, string, or chain of ZF motifs arranged in tandem. A ZFA can have 1, 2, 3, 4, 5, 6, 7, 8, 9, 10, 11, 12, 13, 14, or 15 zinc finger motifs. The ZFA can have from 1-10, 1-15, 1-2, 1-3, 1-4, 1-5, 1-6, 1-7, 1-8, 1-9, 2-3, 2-4, 2-5, 2-6, 2-7, 2-8, 2-9, 2-10, 3-4, 3-5 3-6, 3-7, 3-8, 3-9, 3-10, 4-5, 4-6, 4-7, 4-8, 4-9, 4-10, 5-6, 5-7, 5-8, 5-9, 5-10, or 5-15 zinc finger motifs. In some embodiments, the ZF protein domain includes 1-10 zinc finger motifs. In some embodiments, the ZF protein domain includes six zinc finger motifs. An exemplary zinc finger (ZF) protein domain that includes six zinc finger motifs is provided as SEQ ID NO: 1. The ZF protein domain can have 1, 2, 3, 4, 5, 6, 7, 8, 9, 10, 11, 12, 13, 14, 15, or more ZFAs. The ZF domain can have from 1-10, 1-15, 1-2, 1-3, 1-4, 1-5, 1-6, 1-7, 1-8, 1-9, 2-3, 2-4, 2-5, 2-6, 2-7, 2-8, 2-9, 2-10, 3-4, 3-5 3-6, 3-7, 3-8, 3-9, 3-10, 4-5, 4-6, 4-7, 4-8, 4-9, 4-10, 5-6, 5-7, 5-8, 5-9, 5-10, 5-15, 10-15, 10-20, or 10-25 ZFAs.

“Transcriptional effector domain” as used herein refers to a polypeptide domain that, when targeted to a promoter region of a gene, is capable of modulating the transcription of the gene. In some embodiments, a DNA binding domain present within the same polypeptide molecule as the transcriptional effector domain, or a DNA binding domain present within the same quaternary structure (e.g., oligomerized monomers) as the transcriptional effector domain may allow for targeting to the promoter region of the gene.

A transcriptional effector domain can include a transcriptional activator domain. “Transcriptional activator domain” as used herein refers to a polypeptide domain that, when targeted to a promoter region of a gene, is capable of activating or increasing the level of transcription of the gene. Examples of transcriptional activators include a Herpes Simplex Virus Protein 16 (VP16) activation domain; an activation domain comprising four tandem copies of VP16; a VP64 activation domain; a p65 activation domain of NFκB (“p65”); an Epstein-Barr virus R transactivator (Rta) activation domain; a tripartite activator comprising the VP64, the p65, and the Rta activation domains (VPR activation domain); a tripartite activator comprising the VP64, the p65, and the HSF1 activation domains (VPH activation domain); and a histone acetyltransferase (HAT) core domain of the human E1A-associated protein p300 (p300 HAT core activation domain). In some embodiments, a transcriptional activator domain, when targeted to a promoter region of a gene, is capable of increasing the transcription level of the gene by at least 10%, at least 20%, at least 30%, at least 40%, at least 50%, at least 60%, at least 70%, at least 80%, at least 90%, at least 100%, at least 250%, at least 300%, at least 350%, at least 400%, at least at 450%, or at least 500%, as compared to the transcription level of the gene in the absence of the transcriptional modulator domain. In some embodiments, a transcriptional activator domain, when targeted to a promoter region of a gene, is capable of increasing the transcription level of the gene by 10%-50%, 10%-100%, 10%-100%, 10%-200%, 10%-300%, 10%-400%, 10%-500%, 50%-500%, 100%-500%, 200%-500%, as compared to the transcription level of the gene in the absence of the transcriptional modulator domain.

A transcriptional effector domain can include a transcriptional repressor domain. “Transcriptional repressor domain” as used herein refers to a polypeptide domain that, when targeted to a promoter region of a gene, is capably of inhibiting or decreasing the level of the transcription of the gene. Examples of transcriptional repressors include a Krüppel associated box (KRAB) repression domain; a Histone Deacetylase 4 (HDAC4) repressor domain; a Scleraxis (SCX) HLH domain, an Inhibitor of DNA binding 1 (ID1) HLH domain, a HECT domain and RCC1-like domain-containing protein 2 (HERC2) Cyt-b5 domain, a Twist-related protein 1 (TWST1) HLH domain, an Homeobox protein Nkx-2.2 (NKX22) homeodomain, an Inhibitor of DNA binding 1 (ID3) HLH domain, and a Twist-related protein 2 (TWST2) HLH domain, an EED repressor domain; a Repressor Element Silencing Transcription Factor (REST) repression domain; a WRPW motif (SEQ ID NO: 76) of the hairy-related basic helix-loop-helix repressor proteins, the motif is known as a WRPW (SEQ ID NO: 76) repression domain; a DNA (cytosine-5)-methyltransferase 3B (DNMT3B) repression domain; and an HP1 alpha chromoshadow repression domain.

In some embodiments, a transcriptional repressor domain, when targeted to a promoter region of a gene, is capable of decreasing the transcription level of the gene by at least 10%, at least 20%, at least 30%, at least 40%, at least 50%, at least 60%, at least 70%, at least 80%, at least 90%, at least 100%, at least 250%, at least 300%, at least 350%, at least 400%, at least at 450%, or at least 500%, as compared to the transcription level of the gene in the absence of the transcriptional repressor domain. In some embodiments, a transcriptional repressor domain, when targeted to a promoter region of a gene, is capable of decreasing the transcription level of the gene by 10%-50%, 10%-100%, 10%-100%, 10%-200%, 10%-300%, 10%-400%, 10%-500%, 50%-500%, 100%-500%, 200%-500%, as compared to the transcription level of the gene in the absence of the transcriptional repressor domain.

“Nuclear localization signal” or “NLS” as used herein refers to an amino acid sequence motif present in a protein sequence, which signals to a cell to import the protein into the nucleus by nuclear transport. An NLS typically includes a surface-exposed, short amino acid sequence that is rich in positively charged residues (e.g., lysine or arginine). Examples of NLS include the amino acid sequences of SEQ ID NO: 2-4.

“Nuclear export signal” or “NES” as used herein refers to an amino acid sequence motif, typically about 15 residues long and including 4 hydrophobic residues, that targets the protein for export from the cell nucleus to the cytoplasm through the nuclear pore complex using nuclear transport. Example of NES include the amino acid sequences of SEQ ID NO: 5-14.

The ITFs described herein may include a peptide linker separating two domains. In general, any two domains can be linked together with a peptide linker, and ITFs featuring more than two domains can include more than one linker. Selection of an appropriate linker sequence linking two domains is within the skill of an artisan. A peptide linker can be any polypeptide sequence that separates two polypeptide domains (e.g., an inducible transcription factor and a degron) without interfering with the function of the polypeptide domains. Examples of peptide linkers include GSGSGSGS (SEQ ID NO: 15), KEGS (SEQ ID NO: 16), EGK, EAAAK (SEQ ID NO: 17), AAPAKQE (SEQ ID NO: 18), GSGSGSGSGGAEAAAKEAAAKEAAAKA (SEQ ID NO: 19, referred to herein as “Concatenated Max Jen linker” or “ConMJ”), AAPAKQEAAAPAKQEAAAPAKQEAAAPAPAAKAEAPAAAPAAKA (SEQ ID NO: 20, referred to herein as “ecpd”), and AEAAAKEAAAKEAAAKA (SEQ ID NO: 21).

The term “expression cassette” refers to a polynucleotide generated recombinantly or synthetically, with a series of nucleic acid elements that permit transcription of a particular polynucleotide in a target cell. The expression cassettes described herein can be incorporated into a plasmid, chromosome, mitochondrial DNA, plastid DNA, virus, or nucleic acid fragment. Typically, the expression cassette portion of an expression vector includes, among other sequences, a nucleic acid sequence to be transcribed and a promoter.

As used herein, a “promoter” refers to a control region of a nucleic acid sequence at which initiation and rate of transcription of the remainder of a nucleic acid sequence are controlled. A promoter may also contain sub-regions at which regulatory proteins and molecules may bind, such as RNA polymerase and other transcription factors. Promoters may be constitutive, inducible, repressible, tissue-specific or any combination thereof. A promoter drives expression or drives transcription of the nucleic acid sequence that it regulates. Herein, a promoter is considered to be “operably linked” when it is in a correct functional location and orientation in relation to a nucleic acid sequence it regulates to control (“drive”) transcriptional initiation and/or expression of that sequence.

A promoter may be one naturally associated with a gene or sequence, as may be obtained by isolating the 5′ non-coding sequences located upstream of the coding segment of a given gene or sequence. Such a promoter can be referred to as “endogenous.” In some embodiments, a coding nucleic acid sequence may be positioned under the control of a recombinant or heterologous promoter, which refers to a promoter that is not normally associated with the encoded sequence in its natural environment. Such promoters may include promoters of other genes; promoters isolated from any other cell; and synthetic promoters or enhancers that are not “naturally occurring” such as, for example, those that contain different elements of different transcriptional regulatory regions and/or mutations that alter expression through methods of genetic engineering that are known in the art. In addition to producing nucleic acid sequences of promoters and enhancers synthetically, sequences may be produced using recombinant cloning and/or nucleic acid amplification technology, including polymerase chain reaction (PCR) (see, e.g., U.S. Pat. Nos. 4,683,202 and 5,928,906).

As used herein, a “regulatable promoter” refers to a promoter whose ability to initiate/activate or inhibit/repress transcriptional activity is regulated by a signal. The signal may be an endogenous or a normally exogenous condition (e.g., light), a compound (e.g., a chemical or non-chemical compound) or a protein (e.g., an ITF as described herein) that contacts a regulatable promoter in such a way as to modulate (e.g., induce or repress) promoter activity. For example, an ITF-responsive promoter is a regulatable promoter that can be regulated by an ITF.

As used herein, an “inducible promoter” refers to a promoter whose ability to initiate or activate transcriptional activity is regulated by a signal. The signal may be endogenous or a normally exogenous condition (e.g., light), a compound (e.g., a chemical or non-chemical compound) or a protein (e.g., an ITF including a transcriptional activation domain as described herein) that contacts an inducible promoter in such a way as to activate transcriptional activity from the inducible promoter. For example, an ITF-responsive promoter can be an inducible promoter that is activatable by an ITF.

As used herein, a “repressible promoter” refers to a promoter whose ability to inhibit or repress transcriptional activity is regulated by a signal. The signal may be endogenous or a normally exogenous condition (e.g., light), a compound (e.g., a chemical or non-chemical compound) or a protein (e.g., an ITF including a transcriptional repressor domain as described herein) that contacts a repressible promoter in such a way as to be active in repressing transcriptional activity from the repressible promoter. For example, an ITF-responsive promoter can be a repressible promoter that is repressible by an ITF.

As used herein, a promoter is “responsive to” or “modulated by” or “regulated by” a signal if in the presence of that signal, transcription from the promoter is activated, deactivated, increased, or decreased. In some embodiments, the promoter comprises a response element. A “response element” is a short sequence of DNA within a promoter region that binds specific molecules (e.g., transcription factors) that modulate (regulate) gene expression from the promoter. Response elements that may be used in accordance with the present disclosure include, without limitation, a phloretin-adjustable control element (PEACE), a zinc-finger DNA binding domain (DBD), an interferon-gamma-activated sequence (GAS) (Decker, T. et al. J Interferon Cytokine Res. 1997 March; 17(3): 121-34, incorporated herein by reference), an interferon-stimulated response element (ISRE) (Han, K. J. et al. J Biol Chem. 2004 Apr. 9; 279(15): 15652-61, incorporated herein by reference), a NF-kappaB response element (Wang, V. et al. Cell Reports. 2012; 2(4): 824-839, incorporated herein by reference), and a STAT3 response element (Zhang, D. et al. J of Biol Chem. 1996; 271: 9503-9509, incorporated herein by reference). Other response elements are encompassed herein. Response elements can also contain tandem repeats (e.g., consecutive repeats of the same nucleotide sequence encoding the response element) to generally increase sensitivity of the response element to its cognate binding molecule. Tandem repeats can be labeled 2×, 3×, 4×, 5×, etc. to denote the number of repeats present.

Non-limiting examples of regulatable promoters (e.g., inducible promoters and repressible promoters) are listed in Table 1, which shows the design of the promoter and transcription factor, as well as the effect of the inducer molecule towards the transcription factor (TF) and transgene transcription (T) is shown (B, binding; D, dissociation; n.d., not determined) (A, activation; DA, deactivation; DR, derepression) (see Horner, M. & Weber, W. FEBS Letters 586 (2012) 20784-2096m, and references cited therein). Non-limiting examples of components of regulatable promoters include those shown in Table 2.

TABLE 1

| | | | | Response to | |
|---|---|---|---|---|---|
| | Promoter and | Transcription | Inducer | inducer | |
| System | operator | factor (TF) | molecule | TF | T |

Transcriptional activator-responsive promoters

| | | | | | |
|---|---|---|---|---|---|
| AIR | PAIR (OalcA- | AlcR | Acetaldehyde | n.d. | A |
| | PhCMVmin) | | | | |
| ART | PART (OARG- | ArgR-VP16 | 1-Arginine | B | A |
| | PhCMVmin) | | | | |
| BIT | PBIT3 (OBirA3- | BIT (BirA- | Biotin | B | A |
| | PhCMVmin) | VP16) | | | |
| Cumate - | PCR5 (OCuO6- | cTA (CymR- | Cumate | D | DA |
| activator | PhCMVmin) | VP16) | | | |
| Cumate - | PCR5 (OCuO6- | rcTA (rCymR- | Cumate | B | A |
| reverse activator | PhCMVmin) | VP16) | | | |
| E-OFF | PETR (OETR- | ET (E-VP16) | Erythromycin | D | DA |
| | PhCMVmin) | | | | |
| NICE-OFF | PNIC (ONIC- | NT (HdnoR- | 6-Hydroxy- | D | DA |
| | PhCMVmin) | VP16) | nicotine | | |
| PEACE | PTtgR1 (OTtgR- | TtgA1 (TtgR- | Phloretin | D | DA |
| | PhCMVmin) | VP16) | | | |
| PIP-OFF | PPIR (OPIR- | PIT (PIP- | Pristinamycin I | D | DA |
| | Phsp70 min) | VP16) | | | |
| QuoRex | PSCA (OscbR- | SCA (ScbR- | SCB1 | D | DA |
| | PhCMVmin)PSPA | VP16) | | | |
| | (OpapRI- | | | | |
| | PhCMVmin) | | | | |
| Redox | PROP (OROP- | REDOX | NADH | D | DA |
| | PhCMVmin) | (REX-VP16) | | | |
| TET-OFF | PhCMV*-1 | tTA (TetR- | Tetracycline | D | DA |
| | (OtetO7- | VP16) | | | |
| | PhCMVmin) | | | | |
| TET-ON | PhCMV*-1 | rtTA (rTetR- | Doxycycline | B | A |
| | (OtetO7- | VP16) | | | |
| | PhCMVmin) | | | | |
| TIGR | PCTA (OrheO- | CTA (RheA- | Heat | D | DA |
| | PhCMVmin) | VP16) | | | |
| TraR | O7x(tra box)- | p65-TraR | 3-Oxo-C8- | B | A |
| | PhCMVmin | | HSL | | |
| VAC-OFF | P1VanO2 | VanA1 (VanR- | Vanillic acid | D | DA |
| | (OVanO2- | VP16) | | | |
| | PhCMVmin) | | | | |

Transcriptional repressor-responsive promoters

| | | | | | |
|---|---|---|---|---|---|
| Cumate - | PCuO (PCMV5- | CymR | Cumate | D | DR |
| repressor | OCuO) | | | | |
| E-ON | PETRON8 | E-KRAB | Erythromycin | D | DR |
| | (PSV40-OETR8) | | | | |
| NICE-ON | PNIC (PSV40- | NS (HdnoR- | 6-Hydroxy- | D | DR |
| | ONIC8) | KRAB) | nicotine | | |
| PIP-ON | PPIRON (PSV40- | PIT3 (PIP- | Pristinamycin I | D | DR |
| | OPIR3) | KRAB) | | | |
| Q-ON | PSCAON8 | SCS (ScbR- | SCB1 | D | DR |
| | (PSV40-OscbR8) | KRAB) | | | |
| TET- | OtetO-PHPRT | tTS-H4 (TetR- | Doxycycline | D | DR |
| ON<comma> | | HDAC4) | | | |
| repressor-based | | | | | |
| T-REX | PTetO (PhCMV- | TetR | Tetracycline | D | DR |
| | OtetO2) | | | | |
| UREX | PUREX8 (PSV40- | mUTS | Uric acid | D | DR |
| | OhucO8) | (KRAB-HucR) | | | |
| VAC-ON | PVanON8 | VanA4 (VanR- | Vanillic acid | D | DR |
| | (PhCMV-OVanO8) | KRAB) | | | |

Hybrid promoters

| | | | | | |
|---|---|---|---|---|---|
| QuoRexPIP- | OscbR8-OPIR3- | SCAPIT3 | SCB1 | D | DA |
| ON(NOT IF | PhCMVmin | | Pristinamycin I | D | DR |
| gate) | | | | | |
| QuoRexE- | OscbR-OETR8- | SCAE-KRAB | SCB1 | D | DA |
| ON(NOT IF | PhCMVmin | | Erythromycin | D | DR |
| gate) | | | | | |
| TET-OFFE- | OtetO7-OETR8- | tTAE-KRAB | Tetracycline | D | DA |
| ON(NOT IF | PhCMVmin | | Erythromycin | D | DR |
| gate) | | | | | |
| TET-OFFPIP- | OtetO7-OPIR3- | tTAPIT3E- | Tetracycline | D | DA |
| ONE-ON | OETR8- | KRAB | Pristinamycin I | D | DR |
| | PhCMVmin | | Erythromycin | D | DR |

TABLE 2

| Name | DNA SEQUENCE | Source |
|---|---|---|
| minimal promoter; | AGAGGGTATATAATGGAAGCTCGA | EU581860.1 |
| minP | CTTCCAG (SEQ ID NO: 22) | (Promega) |
| NFKB response element | GGGAATTTCCGGGGACTTTCCGGGA | EU581860.1 |
| protein promoter; 5x | ATTTCCGGGGACTTTCCGGGAATTT | (Promega) |
| NFKB-RE | CC (SEQ ID NO: 23) | |
| CREB response element | CACCAGACAGTGACGTCAGCTGCC | DQ904461.1 |
| protein promoter; 4x | AGATCCCATGGCCGTCATACTGTGA | (Promega) |
| CRE | CGTCTTTCAGACACCCCATTGACGT | |
| | CAATGGGAGAA (SEQ ID NO: 24) | |
| NFAT response element | GGAGGAAAAACTGTTTCATACAGA | DQ904462.1 |
| protein promoter; 3x | AGGCGTGGAGGAAAAACTGTTTCA | (Promega) |
| NFAT binding sites | TACAGAAGGCGTGGAGGAAAAACT | |
| | GTTTCATACAGAAGGCGT (SEQ ID NO: 25) | |
| SRF response element | AGGATGTCCATATTAGGACATCTAG | FJ773212.1 |
| protein promoter; 5x | GATGTCCATATTAGGACATCTAGGA | (Promega) |
| SRE | TGTCCATATTAGGACATCTAGGATG | |
| | TCCATATTAGGACATCTAGGATGTC | |
| | CATATTAGGACATCT (SEQ ID NO: 26) | |
| SRF response element | AGTATGTCCATATTAGGACATCTAC | FJ773213.1 |
| protein promoter 2; 5x | CATGTCCATATTAGGACATCTACTA | (Promega) |
| SRF-RE | TGTCCATATTAGGACATCTTGTATG | |
| | TCCATATTAGGACATCTAAAATGTC | |
| | CATATTAGGACATCT (SEQ ID NO: 27) | |
| AP1 response element | TGAGTCAGTGACTCAGTGAGTCAGT | JQ858516.1 |
| protein promoter; 6x | GACTCAGTGAGTCAGTGACTCAG | (Promega) |
| AP1-RE | (SEQ ID NO: 28) | |
| TCF-LEF response | AGATCAAAGGGTTTAAGATCAAAG | JX099537.1 |
| element promoter; 8x | GGCTTAAGATCAAAGGGTATAAGA | (Promega) |
| TCF-LEF-RE | TCAAAGGGCCTAAGATCAAAGGGA | |
| | CTAAGATCAAAGGGTTTAAGATCA | |
| | AAGGGCTTAAGATCAAAGGGCCTA | |
| | (SEQ ID NO: 29) | |
| SBEx4 | GTCTAGACGTCTAGACGTCTAGACG | Addgene Cat No: |
| | TCTAGAC (SEQ ID NO: 30) | 16495 |
| SMAD2/3-CAGACA | CAGACACAGACACAGACACAGACA | Jonk et al. (J Biol |
| x4 | (SEQ ID NO: 31) | Chem. 1998 Aug |
| | | 14; 273(33): 21145-52. |
| STAT3 binding site | Ggatccggtactcgagatctgcgatctaagtaagcttggc | Addgene Sequencing |
| | attccggtactgttggtaaagccac (SEQ ID NO: | Result #211335 |
| | 32) | |

As used herein, a “constitutive promoter” refers to a promoter that generally allows for continual or un-regulated transcriptional activity.

Non-limiting examples of constitutive promoters include the cytomegalovirus (CMV) promoter, the elongation factor 1-alpha (EF1a) promoter and EF1a variants hEF1aV1 and hEF1aV2, the elongation factor (EFS) promoter, the MND promoter (a synthetic promoter that contains the U3 region of a modified MoMuLV LTR with myeloproliferative sarcoma virus enhancer), the phosphoglycerate kinase (PGK) promoter, the spleen focus-forming virus (SFFV) promoter, the simian virus 40 (SV40) promoter, and the ubiquitin C (UbC) promoter. Examples of constitutive promoter nucleic acid sequences are shown in Table 3.

TABLE 3

| Name | DNA SEQUENCE |
|---|---|
| CMV | GTTGACATTGATTATTGACTAGTTATTAATAGTAATCAATTACGGGGTCA |
| | TTAGTTCATAGCCCATATATGGAGTTCCGCGTTACATAACTTACGGTAA |
| | ATGGCCCGCCTGGCTGACCGCCCAACGACCCCCGCCCATTGACGTCAAT |
| | AATGACGTATGTTCCCATAGTAACGCCAATAGGGACTTTCCATTGACGT |
| | CAATGGGTGGAGTATTTACGGTAAACTGCCCACTTGGCAGTACATCAAG |
| | TGTATCATATGCCAAGTACGCCCCCTATTGACGTCAATGACGGTAAATG |
| | GCCCGCCTGGCATTATGCCCAGTACATGACCTTATGGGACTTTCCTACTT |
| | GGCAGTACATCTACGTATTAGTCATCGCTATTACCATGGTGATGCGGTTT |
| | TGGCAGTACATCAATGGGCGTGGATAGCGGTTTGACTCACGGGGATTTC |
| | CAAGTCTCCACCCCATTGACGTCAATGGGAGTTTGTTTTGGCACCAAAA |
| | TCAACGGGACTTTCCAAAATGTCGTAACAACTCCGCCCCATTGACGCAA |
| | ATGGGCGGTAGGCGTGTACGGTGGGAGGTCTATATAAGCAGAGCTC |
| | (SEQ ID NO: 33) |
| EF1a | GGCTCCGGTGCCCGTCAGTGGGCAGAGCGCACATCGCCCACAGTCCCCG |
| | AGAAGTTGGGGGGAGGGGTCGGCAATTGAACCGGTGCCTAGAGAAGGT |
| | GGCGCGGGGTAAACTGGGAAAGTGATGCCGTGTACTGGCTCCGCCTTTT |
| | TCCCGAGGGTGGGGGAGAACCGTATATAAGTGCAGTAGTCGCCGTGAA |
| | CGTTCTTTTTCGCAACGGGTTTGCCGCCAGAACACAGGTAAGTGCCGTG |
| | TGTGGTTCCCGCGGGCCTGGCCTCTTTACGGGTTATGGCCCTTGCGTGCC |
| | TTGAATTACTTCCACCTGGCTGCAGTACGTGATTCTTGATCCCGAGCTTC |
| | GGGTTGGAAGTGGGTGGGAGAGTTCGAGGCCTTGCGCTTAAGGAGCCC |
| | CTTCGCCTCGTGCTTGAGTTGAGGCCTGGCCTGGGCGCTGGGGCCGCCG |
| | CGTGCGAATCTGGTGGCACCTTCGCGCCTGTCTCGCTGCTTTCGATAAGT |
| | CTCTAGCCATTTAAAATTTTTGATGACCTGCTGCGACGCTTTTTTTCTGG |
| | CAAGATAGTCTTGTAAATGCGGGCCAAGATCTGCACACTGGTATTTCGG |
| | TTTTTGGGGCCGCGGGCGGCGACGGGGCCCGTGCGTCCCAGCGCACATG |
| | TTCGGCGAGGCGGGGCCTGCGAGCGCGACCACCGAGAATCGGACGGGG |
| | GTAGTCTCAAGCTGGCCGGCCTGCTCTGGTGCCTGTCCTCGCGCCGCCGT |
| | GTATCGCCCCGCCCCGGGCGGCAAGGCTGGCCCGGTCGGCACCAGTTGC |
| | GTGAGCGGAAAGATGGCCGCTTCCCGGTCCTGCTGCAGGGAGCTCAAA |
| | ATGGAGGACGCGGCGCTCGGGAGAGCGGGCGGGTGAGTCACCCACACA |
| | AAGGAAAAGGGCCTTTCCGTCCTCAGCCGTCGCTTCATGTGACTCCACG |
| | GAGTACCGGGCGCCGTCCAGGCACCTCGATTAGTTCTCGAGCTTTTGGA |
| | GTACGTCGTCTTTAGGTTGGGGGGAGGGGTTTTATGCGATGGAGTTTCC |
| | CCACACTGAGTGGGTGGAGACTGAAGTTAGGCCAGCTTGGCACTTGATG |
| | TAATTCTCCTTGGAATTTGCCCTTTTTGAGTTTGGATCTTGGTTCATTCTC |
| | AAGCCTCAGACAGTGGTTCAAAGTTTTTTTCTTCCATTTCAGGTGTCGTG |
| | A (SEQ ID NO: 34) |
| EFS | GGATCTGCGATCGCTCCGGTGCCCGTCAGTGGGCAGAGCGCACATCGCC |
| | CACAGTCCCCGAGAAGTTGGGGGGAGGGGTCGGCAATTGAACCGGTGC |
| | CTAGAGAAGGTGGCGCGGGGTAAACTGGGAAAGTGATGTCGTGTACTG |
| | GCTCCGCCTTTTTCCCGAGGGTGGGGGAGAACCGTATATAAGTGCAGTA |
| | GTCGCCGTGAACGTTCTTTTTCGCAACGGGTTTGCCGCCAGAACACAGC |
| | TGAAGCTTCGAGGGGCTCGCATCTCTCCTTCACGCGCCCGCCGCCCTAC |
| | CTGAGGCCGCCATCCACGCCGGTTGAGTCGCGTTCTGCCGCCTCCCGCC |
| | TGTGGTGCCTCCTGAACTGCGTCCGCCGTCTAGGTAAGTTTAAAGCTCA |
| | GGTCGAGACCGGGCCTTTGTCCGGCGCTCCCTTGGAGCCTACCTAGACT |
| | CAGCCGGCTCTCCACGCTTTGCCTGACCCTGCTTGCTCAACTCTACGTCT |
| | TTGTTTCGTTTTCTGTTCTGCGCCGTTACAGATCCAAGCTGTGACCGGCG |
| | CCTAC (SEQ ID NO: 35) |
| MND | TTTATTTAGTCTCCAGAAAAAGGGGGGAATGAAAGACCCCACCTGTAGG |
| | TTTGGCAAGCTAGGATCAAGGTTAGGAACAGAGAGACAGCAGAATATG |
| | GGCCAAACAGGATATCTGTGGTAAGCAGTTCCTGCCCCGGCTCAGGGCC |
| | AAGAACAGTTGGAACAGCAGAATATGGGCCAAACAGGATATCTGTGGT |
| | AAGCAGTTCCTGCCCCGGCTCAGGGCCAAGAACAGATGGTCCCCAGATG |
| | CGGTCCCGCCCTCAGCAGTTTCTAGAGAACCATCAGATGTTTCCAGGGT |
| | GCCCCAAGGACCTGAAATGACCCTGTGCCTTATTTGAACTAACCAATCA |
| | GTTCGCTTCTCGCTTCTGTTCGCGCGCTTCTGCTCCCCGAGCTCAATAAA |
| | AGAGCCCA (SEQ ID NO: 36) |
| PGK | GGGGTTGGGGTTGCGCCTTTTCCAAGGCAGCCCTGGGTTTGCGCAGGGA |
| | CGCGGCTGCTCTGGGCGTGGTTCCGGGAAACGCAGCGGCGCCGACCCTG |
| | GGTCTCGCACATTCTTCACGTCCGTTCGCAGCGTCACCCGGATCTTCGCC |
| | GCTACCCTTGTGGGCCCCCCGGCGACGCTTCCTGCTCCGCCCCTAAGTCG |
| | GGAAGGTTCCTTGCGGTTCGCGGCGTGCCGGACGTGACAAACGGAAGC |
| | CGCACGTCTCACTAGTACCCTCGCAGACGGACAGCGCCAGGGAGCAAT |
| | GGCAGCGCGCCGACCGCGATGGGCTGTGGCCAATAGCGGCTGCTCAGC |
| | GGGGCGCGCCGAGAGCAGCGGCCGGGAAGGGGCGGTGCGGGAGGCGG |
| | GGTGTGGGGCGGTAGTGTGGGCCCTGTTCCTGCCCGCGCGGTGTTCCGC |
| | ATTCTGCAAGCCTCCGGAGCGCACGTCGGCAGTCGGCTCCCTCGTTGAC |
| | CGAATCACCGACCTCTCTCCCCAG (SEQ ID NO: 37) |
| SFFV | GTAACGCCATTTTGCAAGGCATGGAAAAATACCAAACCAAGAATAGAG |
| | AAGTTCAGATCAAGGGCGGGTACATGAAAATAGCTAACGTTGGGCCAA |
| | ACAGGATATCTGCGGTGAGCAGTTTCGGCCCCGGCCCGGGGCCAAGAA |
| | CAGATGGTCACCGCAGTTTCGGCCCCGGCCCGAGGCCAAGAACAGATG |
| | GTCCCCAGATATGGCCCAACCCTCAGCAGTTTCTTAAGACCCATCAGAT |
| | GTTTCCAGGCTCCCCCAAGGACCTGAAATGACCCTGCGCCTTATTTGAA |
| | TTAACCAATCAGCCTGCTTCTCGCTTCTGTTCGCGCGCTTCTGCTTCCCG |
| | AGCTCTATAAAAGAGCTCACAACCCCTCACTCGGCGCGCCAGTCCTCCG |
| | ACAGACTGAGTCGCCCGGG (SEQ ID NO: 38) |
| SV40 | CTGTGGAATGTGTGTCAGTTAGGGTGTGGAAAGTCCCCAGGCTCCCCAG |
| | CAGGCAGAAGTATGCAAAGCATGCATCTCAATTAGTCAGCAACCAGGT |
| | GTGGAAAGTCCCCAGGCTCCCCAGCAGGCAGAAGTATGCAAAGCATGC |
| | ATCTCAATTAGTCAGCAACCATAGTCCCGCCCCTAACTCCGCCCATCCC |
| | GCCCCTAACTCCGCCCAGTTCCGCCCATTCTCCGCCCCATGGCTGACTAA |
| | TTTTTTTTATTTATGCAGAGGCCGAGGCCGCCTCTGCCTCTGAGCTATTC |
| | CAGAAGTAGTGAGGAGGCTTTTTTGGAGGCCTAGGCTTTTGCAAAAAGC |
| | T (SEQ ID NO: 39) |
| UbC | GCGCCGGGTTTTGGCGCCTCCCGCGGGCGCCCCCCTCCTCACGGCGAGC |
| | GCTGCCACGTCAGACGAAGGGCGCAGGAGCGTTCCTGATCCTTCCGCCC |
| | GGACGCTCAGGACAGCGGCCCGCTGCTCATAAGACTCGGCCTTAGAACC |
| | CCAGTATCAGCAGAAGGACATTTTAGGACGGGACTTGGGTGACTCTAGG |
| | GCACTGGTTTTCTTTCCAGAGAGCGGAACAGGCGAGGAAAAGTAGTCCC |
| | TTCTCGGCGATTCTGCGGAGGGATCTCCGTGGGGCGGTGAACGCCGATG |
| | ATTATATAAGGACGCGCCGGGTGTGGCACAGCTAGTTCCGTCGCAGCCG |
| | GGATTTGGGTCGCGGTTCTTGTTTGTGGATCGCTGTGATCGTCACTTGGT |
| | GAGTTGCGGGCTGCTGGGCTGGCCGGGGCTTTCGTGGCCGCCGGGCCGC |
| | TCGGTGGGACGGAAGCGTGTGGAGAGACCGCCAAGGGCTGTAGTCTGG |
| | GTCCGCGAGCAAGGTTGCCCTGAACTGGGGGTTGGGGGGAGCGCACAA |
| | AATGGCGGCTGTTCCCGAGTCTTGAATGGAAGACGCTTGTAAGGCGGGC |
| | TGTGAGGTCGTTGAAACAAGGTGGGGGGCATGGTGGGCGGCAAGAACC |
| | CAAGGTCTTGAGGCCTTCGCTAATGCGGGAAAGCTCTTATTCGGGTGAG |
| | ATGGGCTGGGGCACCATCTGGGGACCCTGACGTGAAGTTTGTCACTGAC |
| | TGGAGAACTCGGGTTTGTCGTCTGGTTGCGGGGGCGGCAGTTATGCGGT |
| | GCCGTTGGGCAGTGCACCCGTACCTTTGGGAGCGCGCGCCTCGTCGTGT |
| | CGTGACGTCACCCGTTCTGTTGGCTTATAATGCAGGGTGGGGCCACCTG |
| | CCGGTAGGTGTGCGGTAGGCTTTTCTCCGTCGCAGGACGCAGGGTTCGG |
| | GCCTAGGGTAGGCTCTCCTGAATCGACAGGCGCCGGACCTCTGGTGAGG |
| | GGAGGGATAAGTGAGGCGTCAGTTTCTTTGGTCGGTTTTATGTACCTATC |
| | TTCTTAAGTAGCTGAAGCTCCGGTTTTGAACTATGCGCTCGGGGTTGGC |
| | GAGTGTGTTTTGTGAAGTTTTTTAGGCACCTTTTGAAATGTAATCATTTG |
| | GGTCAATATGTAATTTTCAGTGTTAGACTAGTAAAGCTTCTGCAGGTCG |
| | ACTCTAGAAAATTGTCCGCTAAATTCTGGCCGTTTTTGGCTTTTTTGTTA |
| | GAC (SEQ ID NO: 40) |
| hEF1aV1 | GGCTCCGGTGCCCGTCAGTGGGCAGAGCGCACATCGCCCACAGTCCCCG |
| | AGAAGTTGGGGGGAGGGGTCGGCAATTGAACCGGTGCCTAGAGAAGGT |
| | GGCGCGGGGTAAACTGGGAAAGTGATGTCGTGTACTGGCTCCGCCTTTT |
| | TCCCGAGGGTGGGGGAGAACCGTATATAAGTGCAGTAGTCGCCGTGAA |
| | CGTTCTTTTTCGCAACGGGTTTGCCGCCAGAACACAGGTAAGTGCCGTG |
| | TGTGGTTCCCGCGGGCCTGGCCTCTTTACGGGTTATGGCCCTTGCGTGCC |
| | TTGAATTACTTCCACCTGGCTGCAGTACGTGATTCTTGATCCCGAGCTTC |
| | GGGTTGGAAGTGGGTGGGAGAGTTCGAGGCCTTGCGCTTAAGGAGCCC |
| | CTTCGCCTCGTGCTTGAGTTGAGGCCTGGCCTGGGCGCTGGGGCCGCCG |
| | CGTGCGAATCTGGTGGCACCTTCGCGCCTGTCTCGCTGCTTTCGATAAGT |
| | CTCTAGCCATTTAAAATTTTTGATGACCTGCTGCGACGCTTTTTTTCTGG |
| | CAAGATAGTCTTGTAAATGCGGGCCAAGATCTGCACACTGGTATTTCGG |
| | TTTTTGGGGCCGCGGGCGGCGACGGGGCCCGTGCGTCCCAGCGCACATG |
| | TTCGGCGAGGCGGGGCCTGCGAGCGCGGCCACCGAGAATCGGACGGGG |
| | GTAGTCTCAAGCTGGCCGGCCTGCTCTGGTGCCTGGTCTCGCGCCGCCG |
| | TGTATCGCCCCGCCCTGGGCGGCAAGGCTGGCCCGGTCGGCACCAGTTG |
| | CGTGAGCGGAAAGATGGCCGCTTCCCGGCCCTGCTGCAGGGAGCTCAA |
| | AATGGAGGACGCGGCGCTCGGGAGAGCGGGCGGGTGAGTCACCCACAC |
| | AAAGGAAAAGGGCCTTTCCGTCCTCAGCCGTCGCTTCATGTGACTCCAC |
| | GGAGTACCGGGCGCCGTCCAGGCACCTCGATTAGTTCTCGAGCTTTTGG |
| | AGTACGTCGTCTTTAGGTTGGGGGGAGGGGTTTTATGCGATGGAGTTTC |
| | CCCACACTGAGTGGGTGGAGACTGAAGTTAGGCCAGCTTGGCACTTGAT |
| | GTAATTCTCCTTGGAATTTGCCCTTTTTGAGTTTGGATCTTGGTTCATTCT |
| | CAAGCCTCAGACAGTGGTTCAAAGTTTTTTTCTTCCATTTCAGGTGTCGT |
| | GA (SEQ ID NO: 41) |
| hCAGG | ACTAGTTATTAATAGTAATCAATTACGGGGTCATTAGTTCATAGCCCAT |
| | ATATGGAGTTCCGCGTTACATAACTTACGGTAAATGGCCCGCCTGGCTG |
| | ACCGCCCAACGACCCCCGCCCATTGACGTCAATAATGACGTATGTTCCC |
| | ATAGTAACGCCAATAGGGACTTTCCATTGACGTCAATGGGTGGAGTATT |
| | TACGGTAAACTGCCCACTTGGCAGTACATCAAGTGTATCATATGCCAAG |
| | TACGCCCCCTATTGACGTCAATGACGGTAAATGGCCCGCCTGGCATTAT |
| | GCCCAGTACATGACCTTATGGGACTTTCCTACTTGGCAGTACATCTACGT |
| | ATTAGTCATCGCTATTACCATGGTCGAGGTGAGCCCCACGTTCTGCTTCA |
| | CTCTCCCCATCTCCCCCCCCTCCCCACCCCCAATTTTGTATTTATTTATTT |
| | TTTAATTATTTTGTGCAGCGATGGGGGCGGGGGGGGGGGGGGGGCGCG |
| | CGCCAGGCGGGGCGGGGCGGGGCGAGGGGCGGGGCGGGGCGAGGCGG |
| | AGAGGTGCGGCGGCAGCCAATCAGAGCGGCGCGCTCCGAAAGTTTCCTT |
| | TTATGGCGAGGCGGCGGCGGCGGCGGCCCTATAAAAAGCGAAGCGCGC |
| | GGCGGGCGGGGAGTCGCTGCGACGCTGCCTTCGCCCCGTGCCCCGCTCC |
| | GCCGCCGCCTCGCGCCGCCCGCCCCGGCTCTGACTGACCGCGTTACTCC |
| | CACAGGTGAGCGGGCGGGACGGCCCTTCTCCTCCGGGCTGTAATTAGCG |
| | CTTGGTTTAATGACGGCTTGTTTCTTTTCTGTGGCTGCGTGAAAGCCTTG |
| | AGGGGCTCCGGGAGGGCCCTTTGTGCGGGGGGAGCGGCTCGGGGGGTG |
| | CGTGCGTGTGTGTGTGCGTGGGGAGCGCCGCGTGCGGCTCCGCGCTGCC |
| | CGGCGGCTGTGAGCGCTGCGGGCGCGGCGCGGGGCTTTGTGCGCTCCGC |
| | AGTGTGCGCGAGGGGAGCGCGGCCGGGGGCGGTGCCCCGCGGTGCGGG |
| | GGGGGCTGCGAGGGGAACAAAGGCTGCGTGCGGGGTGTGTGCGTGGGG |
| | GGGTGAGCAGGGGGTGTGGGCGCGTCGGTCGGGCTGCAACCCCCCCTG |
| | CACCCCCCTCCCCGAGTTGCTGAGCACGGCCCGGCTTCGGGTGCGGGGC |
| | TCCGTACGGGGCGTGGCGCGGGGCTCGCCGTGCCGGGCGGGGGGTGGC |
| | GGCAGGTGGGGGTGCCGGGCGGGGCGGGGCCGCCTCGGGCCGGGGAGG |
| | GCTCGGGGGAGGGGCGCGGCGGCCCCCGGAGCGCCGGCGGCTGTCGAG |
| | GCGCGGCGAGCCGCAGCCATTGCCTTTTATGGTAATCGTGCGAGAGGGC |
| | GCAGGGACTTCCTTTGTCCCAAATCTGTGCGGAGCCGAAATCTGGGAGG |
| | CGCCGCCGCACCCCCTCTAGCGGGCGCGGGGCGAAGCGGTGCGGCGCC |
| | GGCAGGAAGGAAATGGGCGGGGAGGGCCTTCGTGCGTCGCCGCGCCGC |
| | CGTCCCCTTCTCCCTCTCCAGCCTCGGGGCTGTCCGCGGGGGGACGGCT |
| | GCCTTCGGGGGGGACGGGGCAGGGCGGGGTTCGGCTTCTGGCGTGTGA |
| | CCGGCGGCTCTAGAGCCTCTGCTAACCATGTTCATGCCTTCTTCTTTTTC |
| | CTACAGCTCCTGGGCAACGTGCTGGTTATTGTGCTGTCTCATCATTTTGG |
| | CAAAGAATTC (SEQ ID NO: 42) |
| hEF1aV2 | GGGCAGAGCGCACATCGCCCACAGTCCCCGAGAAGTTGGGGGGAGGGG |
| | TCGGCAATTGAACCGGTGCCTAGAGAAGGTGGCGCGGGGTAAACTGGG |
| | AAAGTGATGTCGTGTACTGGCTCCGCCTTTTTCCCGAGGGTGGGGGAGA |
| | ACCGTATATAAGTGCAGTAGTCGCCGTGAACGTTCTTTTTCGCAACGGG |
| | TTTGCCGCCAGAACACAG (SEQ ID NO: 43) |
| hACTb | CCACTAGTTCCATGTCCTTATATGGACTCATCTTTGCCTATTGCGACACA |
| | CACTCAATGAACACCTACTACGCGCTGCAAAGAGCCCCGCAGGCCTGAG |
| | GTGCCCCCACCTCACCACTCTTCCTATTTTTGTGTAAAAATCCAGCTTCT |
| | TGTCACCACCTCCAAGGAGGGGGAGGAGGAGGAAGGCAGGTTCCTCTA |
| | GGCTGAGCCGAATGCCCCTCTGTGGTCCCACGCCACTGATCGCTGCATG |
| | CCCACCACCTGGGTACACACAGTCTGTGATTCCCGGAGCAGAACGGACC |
| | CTGCCCACCCGGTCTTGTGTGCTACTCAGTGGACAGACCCAAGGCAAGA |
| | AAGGGTGACAAGGACAGGGTCTTCCCAGGCTGGCTTTGAGTTCCTAGCA |
| | CCGCCCCGCCCCCAATCCTCTGTGGCACATGGAGTCTTGGTCCCCAGAG |
| | TCCCCCAGCGGCCTCCAGATGGTCTGGGAGGGCAGTTCAGCTGTGGCTG |
| | CGCATAGCAGACATACAACGGACGGTGGGCCCAGACCCAGGCTGTGTA |
| | GACCCAGCCCCCCCGCCCCGCAGTGCCTAGGTCACCCACTAACGCCCCA |
| | GGCCTGGTCTTGGCTGGGCGTGACTGTTACCCTCAAAAGCAGGCAGCTC |
| | CAGGGTAAAAGGTGCCCTGCCCTGTAGAGCCCACCTTCCTTCCCAGGGC |
| | TGCGGCTGGGTAGGTTTGTAGCCTTCATCACGGGCCACCTCCAGCCACT |
| | GGACCGCTGGCCCCTGCCCTGTCCTGGGGAGTGTGGTCCTGCGACTTCT |
| | AAGTGGCCGCAAGCCACCTGACTCCCCCAACACCACACTCTACCTCTCA |
| | AGCCCAGGTCTCTCCCTAGTGACCCACCCAGCACATTTAGCTAGCTGAG |
| | CCCCACAGCCAGAGGTCCTCAGGCCCTGCTTTCAGGGCAGTTGCTCTGA |
| | AGTCGGCAAGGGGGAGTGACTGCCTGGCCACTCCATGCCCTCCAAGAGC |
| | TCCTTCTGCAGGAGCGTACAGAACCCAGGGCCCTGGCACCCGTGCAGAC |
| | CCTGGCCCACCCCACCTGGGCGCTCAGTGCCCAAGAGATGTCCACACCT |
| | AGGATGTCCCGCGGTGGGTGGGGGGCCCGAGAGACGGGCAGGCCGGGG |
| | GCAGGCCTGGCCATGCGGGGCCGAACCGGGCACTGCCCAGCGTGGGGC |
| | GCGGGGGCCACGGCGCGCGCCCCCAGCCCCCGGGCCCAGCACCCCAAG |
| | GCGGCCAACGCCAAAACTCTCCCTCCTCCTCTTCCTCAATCTCGCTCTCG |
| | CTCTTTTTTTTTTTCGCAAAAGGAGGGGAGAGGGGGTAAAAAAATGCTG |
| | CACTGTGCGGCGAAGCCGGTGAGTGAGCGGCGCGGGGCCAATCAGCGT |
| | GCGCCGTTCCGAAAGTTGCCTTTTATGGCTCGAGCGGCCGCGGCGGCGC |
| | CCTATAAAACCCAGCGGCGCGACGCGCCACCACCGCCGAGACCGCGTC |
| | CGCCCCGCGAGCACAGAGCCTCGCCTTTGCCGATCCGCCGCCCGTCCAC |
| | ACCCGCCGCCAGGTAAGCCCGGCCAGCCGACCGGGGCAGGCGGCTCAC |
| | GGCCCGGCCGCAGGCGGCCGCGGCCCCTTCGCCCGTGCAGAGCCGCCGT |
| | CTGGGCCGCAGCGGGGGGCGCATGGGGGGGGAACCGGACCGCCGTGGG |
| | GGGCGCGGGAGAAGCCCCTGGGCCTCCGGAGATGGGGGACACCCCACG |
| | CCAGTTCGGAGGCGCGAGGCCGCGCTCGGGAGGCGCGCTCCGGGGGTG |
| | CCGCTCTCGGGGCGGGGGCAACCGGCGGGGTCTTTGTCTGAGCCGGGCT |
| | CTTGCCAATGGGGATCGCAGGGTGGGCGCGGCGGAGCCCCCGCCAGGC |
| | CCGGTGGGGGCTGGGGCGCCATTGCGCGTGCGCGCTGGTCCTTTGGGCG |
| | CTAACTGCGTGCGCGCTGGGAATTGGCGCTAATTGCGCGTGCGCGCTGG |
| | GACTCAAGGCGCTAACTGCGCGTGCGTTCTGGGGCCCGGGGTGCCGCGG |
| | CCTGGGCTGGGGCGAAGGCGGGCTCGGCCGGAAGGGGTGGGGTCGCCG |
| | CGGCTCCCGGGCGCTTGCGCGCACTTCCTGCCCGAGCCGCTGGCCGCCC |
| | GAGGGTGTGGCCGCTGCGTGCGCGCGCGCCGACCCGGCGCTGTTTGAAC |
| | CGGGCGGAGGCGGGGCTGGCGCCCGGTTGGGAGGGGGTTGGGGCCTGG |
| | CTTCCTGCCGCGCGCCGCGGGGACGCCTCCGACCAGTGTTTGCCTTTTAT |
| | GGTAATAACGCGGCCGGCCCGGCTTCCTTTGTCCCCAATCTGGGCGCGC |
| | GCCGGCGCCCCCTGGCGGCCTAAGGACTCGGCGCGCCGGAAGTGGCCA |
| | GGGCGGGGGCGACCTCGGCTCACAGCGCGCCCGGCTAT (SEQ ID NO: 44) |
| heIF4A1 | GTTGATTTCCTTCATCCCTGGCACACGTCCAGGCAGTGTCGAATCCATCT |
| | CTGCTACAGGGGAAAACAAATAACATTTGAGTCCAGTGGAGACCGGGA |
| | GCAGAAGTAAAGGGAAGTGATAACCCCCAGAGCCCGGAAGCCTCTGGA |
| | GGCTGAGACCTCGCCCCCCTTGCGTGATAGGGCCTACGGAGCCACATGA |
| | CCAAGGCACTGTCGCCTCCGCACGTGTGAGAGTGCAGGGCCCCAAGATG |
| | GCTGCCAGGCCTCGAGGCCTGACTCTTCTATGTCACTTCCGTACCGGCG |
| | AGAAAGGCGGGCCCTCCAGCCAATGAGGCTGCGGGGGGGGCCTTCACC |
| | TTGATAGGCACTCGAGTTATCCAATGGTGCCTGCGGGCCGGAGCGACTA |
| | GGAACTAACGTCATGCCGAGTTGCTGAGCGCCGGCAGGCGGGGCCGGG |
| | GCGGCCAAACCAATGCGATGGCCGGGGCGGAGTCGGGCGCTCTATAAG |
| | TTGTCGATAGGCGGGCACTCCGCCCTAGTTTCTAAGGACCATG (SEQ ID NO: 45) |
| hGAPDH | AGTTCCCCAACTTTCCCGCCTCTCAGCCTTTGAAAGAAAGAAAGGGGAG |
| | GGGGCAGGCCGCGTGCAGTCGCGAGCGGTGCTGGGCTCCGGCTCCAATT |
| | CCCCATCTCAGTCGCTCCCAAAGTCCTTCTGTTTCATCCAAGCGTGTAAG |
| | GGTCCCCGTCCTTGACTCCCTAGTGTCCTGCTGCCCACAGTCCAGTCCTG |
| | GGAACCAGCACCGATCACCTCCCATCGGGCCAATCTCAGTCCCTTCCCC |
| | CCTACGTCGGGGCCCACACGCTCGGTGCGTGCCCAGTTGAACCAGGCGG |
| | CTGCGGAAAAAAAAAAGCGGGGAGAAAGTAGGGCCCGGCTACTAGCGG |
| | TTTTACGGGCGCACGTAGCTCAGGCCTCAAGACCTTGGGCTGGGACTGG |
| | CTGAGCCTGGCGGGAGGCGGGGTCCGAGTCACCGCCTGCCGCCGCGCCC |
| | CCGGTTTCTATAAATTGAGCCCGCAGCCTCCCGCTTCGCTCTCTGCTCCT |
| | CCTGTTCGACAGTCAGCCGCATCTTCTTTTGCGTCGCCAGGTGAAGACG |
| | GGCGGAGAGAAACCCGGGAGGCTAGGGACGGCCTGAAGGCGGCAGGG |
| | GCGGGCGCAGGCCGGATGTGTTCGCGCCGCTGCGGGGTGGGCCCGGGC |
| | GGCCTCCGCATTGCAGGGGGGGGCGGAGGACGTGATGCGGCGCGGGCT |
| | GGGCATGGAGGCCTGGTGGGGGAGGGGAGGGGAGGCGTGGGTGTCGGC |
| | CGGGGCCACTAGGCGCTCACTGTTCTCTCCCTCCGCGCAGCCGAGCCAC |
| | ATCGCTGAGACAC (SEQ ID NO: 46) |
| hGRP78 | AGTGCGGTTACCAGCGGAAATGCCTCGGGGTCAGAAGTCGCAGGAGAG |
| | ATAGACAGCTGCTGAACCAATGGGACCAGCGGATGGGGCGGATGTTAT |
| | CTACCATTGGTGAACGTTAGAAACGAATAGCAGCCAATGAATCAGCTGG |
| | GGGGGCGGAGCAGTGACGTTTATTGCGGAGGGGGCCGCTTCGAATCGG |
| | CGGCGGCCAGCTTGGTGGCCTGGGCCAATGAACGGCCTCCAACGAGCA |
| | GGGCCTTCACCAATCGGCGGCCTCCACGACGGGGCTGGGGGAGGGTAT |
| | ATAAGCCGAGTAGGCGACGGTGAGGTCGACGCCGGCCAAGACAGCACA |
| | GACAGATTGACCTATTGGGGTGTTTCGCGAGTGTGAGAGGGAAGCGCCG |
| | CGGCCTGTATTTCTAGACCTGCCCTTCGCCTGGTTCGTGGCGCCTTGTGA |
| | CCCCGGGCCCCTGCCGCCTGCAAGTCGGAAATTGCGCTGTGCTCCTGTG |
| | CTACGGCCTGTGGCTGGACTGCCTGCTGCTGCCCAACTGGCTGGCAC |
| | (SEQ ID NO: 47) |
| hGRP94 | TAGTTTCATCACCACCGCCACCCCCCCGCCCCCCCGCCATCTGAAAGGG |
| | TTCTAGGGGATTTGCAACCTCTCTCGTGTGTTTCTTCTTTCCGAGAAGCG |
| | CCGCCACACGAGAAAGCTGGCCGCGAAAGTCGTGCTGGAATCACTTCCA |
| | ACGAAACCCCAGGCATAGATGGGAAAGGGTGAAGAACACGTTGCCATG |
| | GCTACCGTTTCCCCGGTCACGGAATAAACGCTCTCTAGGATCCGGAAGT |
| | AGTTCCGCCGCGACCTCTCTAAAAGGATGGATGTGTTCTCTGCTTACATT |
| | CATTGGACGTTTTCCCTTAGAGGCCAAGGCCGCCCAGGCAAAGGGGCGG |
| | TCCCACGCGTGAGGGGCCCGCGGAGCCATTTGATTGGAGAAAAGCTGC |
| | AAACCCTGACCAATCGGAAGGAGCCACGCTTCGGGCATCGGTCACCGC |
| | ACCTGGACAGCTCCGATTGGTGGACTTCCGCCCCCCCTCACGAATCCTC |
| | ATTGGGTGCCGTGGGTGCGTGGTGCGGCGCGATTGGTGGGTTCATGTTT |
| | CCCGTCCCCCGCCCGCGAGAAGTGGGGGTGAAAAGCGGCCCGACCTGC |
| | TTGGGGTGTAGTGGGCGGACCGCGCGGCTGGAGGTGTGAGGATCCGAA |
| | CCCAGGGGTGGGGGGTGGAGGCGGCTCCTGCGATCGAAGGGGACTTGA |
| | GACTCACCGGCCGCACGTC (SEQ ID NO: 48) |
| hHSP70 | GGGCCGCCCACTCCCCCTTCCTCTCAGGGTCCCTGTCCCCTCCAGTGAAT |
| | CCCAGAAGACTCTGGAGAGTTCTGAGCAGGGGGCGGCACTCTGGCCTCT |
| | GATTGGTCCAAGGAAGGCTGGGGGGCAGGACGGGAGGCGAAAACCCTG |
| | GAATATTCCCGACCTGGCAGCCTCATCGAGCTCGGTGATTGGCTCAGAA |
| | GGGAAAAGGCGGGTCTCCGTGACGACTTATAAAAGCCCAGGGGCAAGC |
| | GGTCCGGATAACGGCTAGCCTGAGGAGCTGCTGCGACAGTCCACTACCT |
| | TTTTCGAGAGTGACTCCCGTTGTCCCAAGGCTTCCCAGAGCGAACCTGT |
| | GCGGCTGCAGGCACCGGCGCGTCGAGTTTCCGGCGTCCGGAAGGACCG |
| | AGCTCTTCTCGCGGATCCAGTGTTCCGTTTCCAGCCCCCAATCTCAGAGC |
| | GGAGCCGACAGAGAGCAGGGAACCC (SEQ ID NO: 49) |
| hKINb | GCCCCACCCCCGTCCGCGTTACAACCGGGAGGCCCGCTGGGTCCTGCAC |
| | CGTCACCCTCCTCCCTGTGACCGCCCACCTGATACCCAAACAACTTTCTC |
| | GCCCCTCCAGTCCCCAGCTCGCCGAGCGCTTGCGGGGAGCCACCCAGCC |
| | TCAGTTTCCCCAGCCCCGGGCGGGGCGAGGGGCGATGACGTCATGCCGG |
| | CGCGCGGCATTGTGGGGCGGGGCGAGGCGGGGCGCCGGGGGGAGCAAC |
| | ACTGAGACGCCATTTTCGGCGGCGGGAGCGGCGCAGGCGGCCGAGCGG |
| | GACTGGCTGGGTCGGCTGGGCTGCTGGTGCGAGGAGCCGCGGGGCTGT |
| | GCTCGGCGGCCAAGGGGACAGCGCGTGGGTGGCCGAGGATGCTGCGGG |
| | GCGGTAGCTCCGGCGCCCCTCGCTGGTGACTGCTGCGCCGTGCCTCACA |
| | CAGCCGAGGCGGGCTCGGCGCACAGTCGCTGCTCCGCGCTCGCGCCCGG |
| | CGGCGCTCCAGGTGCTGACAGCGCGAGAGAGCGCGGCCTCAGGAGCAA |
| | CAC (SEQ ID NO: 50) |
| hUBIb | TTCCAGAGCTTTCGAGGAAGGTTTCTTCAACTCAAATTCATCCGCCTGAT |
| | AATTTTCTTATATTTTCCTAAAGAAGGAAGAGAAGCGCATAGAGGAGAA |
| | GGGAAATAATTTTTTAGGAGCCTTTCTTACGGCTATGAGGAATTTGGGG |
| | CTCAGTTGAAAAGCCTAAACTGCCTCTCGGGAGGTTGGGCGCGGCGAAC |
| | TACTTTCAGCGGCGCACGGAGACGGCGTCTACGTGAGGGGTGATAAGTG |
| | ACGCAACACTCGTTGCATAAATTTGCGCTCCGCCAGCCCGGAGCATTTA |
| | GGGGCGGTTGGCTTTGTTGGGTGAGCTTGTTTGTGTCCCTGTGGGTGGAC |
| | GTGGTTGGTGATTGGCAGGATCCTGGTATCCGCTAACAGGTACTGGCCC |
| | ACAGCCGTAAAGACCTGCGGGGGCGTGAGAGGGGGGAATGGGTGAGGT |
| | CAAGCTGGAGGCTTCTTGGGGTTGGGTGGGCCGCTGAGGGGAGGGGAG |
| | GGCGAGGTGACGCGACACCCGGCCTTTCTGGGAGAGTGGGCCTTGTTGA |
| | CCTAAGGGGGGCGAGGGCAGTTGGCACGCGCACGCGCCGACAGAAACT |
| | AACAGACATTAACCAACAGCGATTCCGTCGCGTTTACTTGGGAGGAAGG |
| | CGGAAAAGAGGTAGTTTGTGTGGCTTCTGGAAACCCTAAATTTGGAATC |
| | CCAGTATGAGAATGGTGTCCCTTCTTGTGTTTCAATGGGATTTTTACTTC |
| | GCGAGTCTTGTGGGTTTGGTTTTGTTTTCAGTTTGCCTAACACCGTGCTT |
| | AGGTTTGAGGCAGATTGGAGTTCGGTCGGGGGAGTTTGAATATCCGGAA |
| | CAGTTAGTGGGGAAAGCTGTGGACGCTTGGTAAGAGAGCGCTCTGGATT |
| | TTCCGCTGTTGACGTTGAAACCTTGAATGACGAATTTCGTATTAAGTGAC |
| | TTAGCCTTGTAAAATTGAGGGGAGGCTTGCGGAATATTAACGTATTTAA |
| | GGCATTTTGAAGGAATAGTTGCTAATTTTGAAGAATATTAGGTGTAAAA |
| | GCAAGAAATACAATGATCCTGAGGTGACACGCTTATGTTTTACTTTTAA |
| | ACTAGGTCACC (SEQ ID NO: 51) |

Transcription Factors that Include Oligomerizable Antibody Domains and/or are Inducible by Caffeine, a Cannabinoid, or Nicotine

In some embodiments, the present disclosure provides an inducible transcription factor (ITF) that includes oligomerizable antibody domains and/or is inducible by caffeine, a cannabinoid (e.g., a phytocannabinoid such as cannabidiol), nicotine, or a derivative thereof.

In some embodiments, the ITF includes a first monomer comprising a DNA binding domain and a first ligand binding domain comprising a first antibody or fragment thereof, and a second monomer comprising a transcription effector domain and a second ligand binding domain comprising a second antibody or fragment thereof. Upon binding to a cognate ligand selected from caffeine, a cannabinoid (e.g., a phytocannabinoid such as cannabidiol), and nicotine, the first antibody or fragment thereof and the second antibody or fragment thereof oligomerize (e.g., dimerize), and ligand-induced oligomerization of the first and second antibodies or fragments thereof forms the inducible transcription factor. In some embodiments, the first and second antibodies or fragments thereof are selected from a VH antibody domain, a VL antibody domain, and a single domain antibody.

In some embodiments, the ITF is inducible by caffeine, a cannabinoid, or nicotine and includes a first monomer comprising a DNA binding domain and a first ligand binding domain, and a second monomer comprising a transcription effector domain and a second ligand binding domain. Upon binding to a cognate ligand selected from caffeine, a cannabinoid (e.g., a phytocannabinoid such as cannabidiol), and nicotine, the first monomer and the second monomer oligomerize (e.g., dimerize), and ligand-induced oligomerization of the first and second monomers forms the inducible transcription factor.

In some embodiments, the cognate ligand is caffeine or a derivative thereof. Caffeine is a central nervous system (CNS) stimulant of the methylxanthine class. Caffeine is classified by the US Food and Drug Administration as generally recognized as safe (GRAS). Toxic doses, over 10 grams per day for an adult, are much higher than the typical dose of under 500 milligrams per day. In some embodiments, the cognate ligand is caffeine and the first ligand binding domain and the second ligand binding domain each specifically bind to caffeine.

In some embodiments, the cognate ligand is a cannabinoid. In some embodiments, the first ligand binding domain and the second ligand binding domain each specifically bind to a cannabinoid (e.g., a phytocannabinoid such as cannabidiol). “Cannabinoid” as used herein refers to any compound that acts on cannabinoid receptors, which are expressed in various parts of the body and are involved in regulating mood, appetite, memory and immune response. Cannabinoids include endocannabinoids, synthetic cannabinoids, and phytocanabinoids. Endocannabinoids are naturally produced by the mammalian body and include anandamide (N-arachidonoylethanolamide) and 2-arachidonoyl glycerol (2-AG). “Synthetic cannabinoid” refers to a compound that interacts with a cannabinoid receptor but is not known to be produced in nature. Examples of synthetic cannabinoids include JWH-018, JWH-073, JWH-398, JWH-250, CP 47,497, and HU-210. “Phytocannabinoid” refers to a cannabinoid that is naturally produced by a plant. Phytocannabinoids include cannabinoids produced by the cannabis plant (e.g., tetrahydrocannabinol, cannabidiol, cannabichromene, and cannabigerol), and cannabinoids produced by additional types of plants such as beta-caryophyllene (produced by plants including cannabis, black pepper, and cloves), and diindolylmethane (produced by cruciferous vegetables including broccoli, cauliflower, cabbage and brussell sprouts). In some embodiments, the cannabinoid is a phytocannabinoid. In some embodiments, the phytocannabinoid is cannabidiol. Cannabidiol (also referred to as “CBD”) is a non-psychoactive cannabis-derived cannabinoid that is used to treat a variety of ailments including pain, insomnia, and anxiety. A typical dose of CBD is in the range of 2 mg to 50 mg. Cannabis flowers produced a molecule known as cannabidiolic acid (CBDA), and upon heat activation becomes decarboxylated to form cannabidiol. In some embodiments, the phytocannabinoid is selected from cannabidiol and cannabidiolic acid. In some embodiments, the cognate ligand is a cannabinoid (e.g., a phytocannabinoid such as CBD) and the first ligand binding domain and the second ligand binding domain each specifically bind to the cannabinoid (e.g., a phytocannabinoid such as CBD).

In some embodiments, the cognate ligand is nicotine or a derivative thereof. Nicotine is a chiral alkaloid that is naturally produced in the nightshade family of plants (most predominantly in tobacco and Duboisia hopwoodii) and is widely used recreationally as a stimulant. A typical dose of nicotine present in a cigarette is about 10 mg to about 12 mg, and about 1 mg to 1.5 mg of nicotine is inhaled during the smoking of a cigarette. In some embodiments, the cognate ligand is nicotine and the first ligand binding domain and the second ligand binding domain each specifically bind to nicotine.

In embodiments, the first ligand binding domain comprises a first antibody or fragment thereof and the second ligand binding domain comprises a second antibody or fragment thereof, wherein the first antibody or fragment thereof and the second antibody or fragment thereof or capable of oligomerizing (e.g., dimerizing) in the presence of a cognate ligand.

In some embodiments, the first ligand binding domain and the second ligand binding domain each comprise a single domain antibody. “Single domain antibody” as used herein (also known as a nanobody), is an antigen-binding fragment derived from a heavy chain-only antibody, such as those present in camelids (V_{H}H, from camels and llamas) and cartilaginous fishes (V_{NAR}, from sharks). In some embodiments, the single domain antibody is a V_{H}H. In some embodiments, the first ligand binding domain comprises a first single domain antibody and the second ligand binding domain comprises a second domain antibody, and the first single domain antibody and the second single domain antibody are capable of dimerizing in the presence of the ligand.

In some embodiments the ITF is a caffeine-inducible transcription factor. In some embodiments, the V_{H}H is an anti-caffeine V_{H}H (also referred to herein as “acV_{H}H”). “Anti-caffeine V_{H}H” refers to a camelid single domain antibody having an antigen binding domain that specifically binds to caffeine.

In some embodiments, the anti-caffeine V_{H}H comprises the amino acid sequence of SEQ ID NO: 52. In some embodiments, the first ligand binding domain comprises a first anti-caffeine V_{H}H (acV_{H}H), and the second ligand binding domain comprises a second anti-caffeine V_{H}H (acV_{H}H). In some embodiments, the first anti-caffeine V_{H}H comprises. In some embodiments, the first monomer and second monomer each comprise the amino acid sequence of SEQ ID NO: 52. In some embodiments, the first anti-caffeine V_{H}H and the second anti-caffeine V_{H}H have an identical amino acid sequence (or share at least 95%, 96%, 96%, 98%, or 99% sequence identity) and are capably of dimerizing in the presence of caffeine, thereby causing the first monomer and the second monomer to dimerize.

In some embodiments, the ITF is a cannabinoid-inducible transcription factor. In some embodiments, the cognate ligand is a cannabinoid (e.g., a phytocannabinoid such as CBD) and the first ligand binding domain and the second ligand binding domain comprise a single domain antibody. In some embodiments, the single domain antibody is V_{H}H. In some embodiments, the V_{H}H is an anti-CBD V_{H}H. Examples of anti-CBD V_{H}H include CA14 (SEQ ID NO: 53), DB6 (SEQ ID NO: 54), DB11 (SEQ ID NO: 55), DB18 (SEQ ID NO: 56), and DB21 (SEQ ID NO: 57). In some embodiments, the V_{H}H comprises an amino acid sequence selected from SEQ ID NO: 53-57. In some embodiments, one of the first or the ligand binding domain comprises the amino acid sequence of SEQ ID NO: 53 (CA-14) and the other of the first or the second ligand binding domain comprises an amino acid sequence selected from SEQ ID NO: 54-57 (DB6, DB11, DB18, and DB21, respectively). In some embodiments, one of the first or the second ligand binding domain comprises the amino acid sequence of SEQ ID NO: 53 (CA-14) and the other of the first or the second ligand binding domain comprises the amino acid sequence of SEQ ID NO: 57 (DB21). In some embodiments, the first anti-CBD V_{H}H and the second anti-CBD V_{H}H are non-identical and are capable of dimerizing in the presence of CBD.

In some embodiments, one of the first or the second ligand binding domain comprises an antibody heavy chain (VH) and the other of the first or the second ligand binding domain comprises an antibody light chain (VL), and the VH and the VL are capable of dimerizing in the presence of the cognate ligand.

In some embodiments, the ITF is a nicotine-inducible transcription factor. In some embodiments, the cognate ligand is nicotine and the first ligand binding domain and the second ligand binding domain each comprise an anti-nicotine antibody heavy chain variable domain (anti-Nic VH) or an anti-nicotine antibody light chain variable domain (anti-Nic VL). In some embodiments, the anti-Nic VH comprises the amino acid sequence of SEQ ID NO: 58. In some embodiments, the anti-Nic VL comprises the amino acid sequence of SEQ ID NO: 59. In some embodiments, one of the first or the second ligand binding domain comprises an anti-Nic VH, and the other of the first or the second ligand binding domain comprises an anti-Nic VL. In some embodiments, the anti-Nic VH and the anti-Nic VL dimerize in the presence of nicotine, thereby causing the first monomer and the second monomer to dimerize.

In some embodiments, the DNA binding domain of the ITF that is inducible by caffeine, a cannabinoid, or nicotine comprises a zinc finger (ZF) protein domain.

In some embodiments, the ZF protein domain is modular in design and is composed of at least one zinc finger array (ZFA).

In some embodiments, the ZF protein domain comprises an array of one to ten zinc finger motifs. In some embodiments, the ZF protein domain comprises six zinc finger motifs. In some embodiments, the ZF protein domain comprises the amino acid sequence of SEQ ID NO: 1.

In some embodiments, the transcriptional effector domain of the ITF that is inducible by caffeine, a cannabinoid, or nicotine is a transcriptional activator domain. In some embodiments, the transcriptional activator domain is selected from: a Herpes Simplex Virus Protein 16 (VP16) activation domain; an activation domain comprising four tandem copies of VP16; a VP64 activation domain; a p65 activation domain of NFκB; an Epstein-Barr virus R transactivator (Rta) activation domain; a tripartite activator comprising the VP64, the p65, and the Rta activation domains (VPR activation domain); and a histone acetyltransferase (HAT) core domain of the human E1A-associated protein p300 (p300 HAT core activation domain).

In some embodiments, the transcriptional effector domain of the ITF that is inducible by caffeine, a cannabinoid, or nicotine is a transcriptional repressor domain. In some embodiments, the transcriptional repressor domain is selected from: a Krüppel associated box (KRAB) repression domain; a truncated Krüppel associated box (KRAB) repression domain; a Histone Deacetylase 4 (HDAC4) repressor domain; a Scleraxis (SCX) HLH domain, an Inhibitor of DNA binding 1 (ID1) HLH domain, a HECT domain and RCC1-like domain-containing protein 2 (HERC2) Cyt-b5 domain, a Twist-related protein 1 (TWST1) HLH domain, an Homeobox protein Nkx-2.2 (NKX22) homeodomain, an Inhibitor of DNA binding 1 (ID3) HLH domain, and a Twist-related protein 2 (TWST2) HLH domain, and EED repressor domain; a Repressor Element Silencing Transcription Factor (REST) repression domain; a WRPW motif (SEQ ID NO: 76) of the hairy-related basic helix-loop-helix repressor proteins, the motif is known as a WRPW (SEQ ID NO: 76) repression domain; a DNA (cytosine-5)-methyltransferase 3B (DNMT3B) repression domain; and an HP1 alpha chromoshadow repression domain.

In some embodiments, the first monomer and/or the second monomer of the ITF that is inducible caffeine, a cannabinoid, or nicotine includes a nuclear localization signal (NLS). In some embodiments, the NLS comprises the amino acid sequence of a sequence selected from SEQ ID NOs: 2-4. In some embodiments, the NLS comprises the amino acid sequence of SEQ ID NO: 2.

In some embodiments, the first monomer of the ITF that is inducible by caffeine, a cannabinoid, or nicotine includes a nuclear localization signal (NLS)

In some embodiments, the first monomer of the ITF that is inducible by caffeine, a cannabinoid, or nicotine includes a nuclear localization signal (NLS), and the second monomer of the ITF that is inducible by rapamycin or a derivative or analog thereof includes a nuclear localization signal (NLS).

In some embodiments, the second monomer of the ITF that is inducible by caffeine, a cannabinoid, or nicotine includes a nuclear export signal (NES). In some embodiments, the NES comprises the amino acid sequence of a sequence selected from SEQ ID NOs: 5-14. In some embodiments, the NES comprises the amino acid sequence of SEQ ID NO: 5.

In some embodiments, the first monomer of the ITF that is inducible by caffeine, a cannabinoid, or nicotine includes a nuclear localization signal (NLS), and the second monomer of the ITF inducible by rapamycin or a derivative or analog thereof includes a nuclear export signal (NES).

In some embodiments, the first monomer of the ITF that is inducible by caffeine, a cannabinoid, or nicotine includes a peptide linker separating the DNA binding domain and the first ligand binding domain. In some embodiments, the DNA binding domain is N-terminal to the first ligand binding domain. In some embodiments, the first ligand binding domain is N-terminal to the DNA binding domain.

In some embodiments, the second monomer of the ITF inducible by caffeine, a cannabinoid, or nicotine includes a peptide linker separating the transcriptional effector domain and the second ligand binding domain. In some embodiments, the transcriptional effector domain is N-terminal to the second ligand binding domain. In some embodiments, the second ligand binding domain is N-terminal to the transcriptional effector domain.

When an ITF that is inducible by caffeine, a cannabinoid, or nicotine is formed by the ligand-induced oligomerization of the first and second monomers, the ITF is capable of modulating transcription of a gene of interest. The oligomerized product of the first and second monomers results in a functional ITF because it the quaternary protein structure of the oligomer includes both the DNA binding domain and the transcriptional effector domain.

### Transcription Factors Inducible by Mifepristone

In some embodiments, the present disclosure provides an inducible transcription factor (ITF) that is inducible by mifepristone or a derivative thereof (i.e., a mifepristone-inducible ITF or “mifepristone-ITF”). The mifepristone-ITF includes a DNA binding domain, a transcription effector domain, and a ligand binding domain. Upon binding to a cognate ligand (e.g., mifepristone or a derivative thereof), the ITF undergoes nuclear localization. When localized to a cell nucleus, the ITF is capable of modulating transcription of a gene of interest operably linked to an ITF-responsive promoter.

Mifepristone (also known as RU486) is an antiprogesterone and antiglucocorticosteroid agent that is used clinically to induce abortion, and also to treat hyperglycemia in patients with Cushing's disease. Mifepristone oral tablets are available in doses of 200 mg and 300 mg.

In some embodiments, the ligand binding domain of the mifepristone-ITF comprises a progesterone receptor domain. Progesterone receptor (also known as NR3C3 or nuclear receptor subfamily 3, group C, member 3) is a nuclear receptor activated by the hormone progesterone. An example of a progesterone receptor sequence is provided as SEQ ID NO: 68. In some embodiments, the ligand binding domain of the mifepristone-ITF comprises the amino acid sequence of SEQ ID NO: 68.

In some embodiments, the DNA binding domain of the mifepristone-ITF comprises a zinc finger (ZF) protein domain.

In some embodiments, the ZF protein domain is modular in design and is composed of zinc finger arrays (ZFA).

In some embodiments, the ZF protein domain comprises one to ten ZFA(s). In some embodiments, the ZF protein domain comprises at least one ZFA. In some embodiments, the ZF protein domain comprises at least two ZFAs. In some embodiments, the ZF protein domain comprises at least three ZFAs. In some embodiments, the ZF protein domain comprises at least four ZFAs. In some embodiments, the ZF protein domain comprises at least five ZFAs. In some embodiments, the ZF protein domain comprises at least ten ZFAs. In some embodiments, the ZF protein domain comprises the amino acid sequence of SEQ ID NO: 1.

In some embodiments, the transcriptional effector domain of the mifepristone-ITF is a transcriptional activator domain. In some embodiments, the transcriptional activator domain is selected from: a Herpes Simplex Virus Protein 16 (VP16) activation domain; an activation domain comprising four tandem copies of VP16; a VP64 activation domain; a p65 activation domain of NFκB; an Epstein-Barr virus R transactivator (Rta) activation domain; a tripartite activator comprising the VP64, the p65, and the Rta activation domains (VPR activation domain); and a histone acetyltransferase (HAT) core domain of the human E1A-associated protein p300 (p300 HAT core activation domain).

In some embodiments, the transcriptional effector domain of the mifepristone-ITF is a transcriptional repressor domain. In some embodiments, the transcriptional repressor domain is selected from: a Krüppel associated box (KRAB) repression domain; a truncated Krüppel associated box (KRAB) repression domain; a Histone Deacetylase 4 (HDAC4) repressor domain; a Scleraxis (SCX) HLH domain, an Inhibitor of DNA binding 1 (ID1) HLH domain, a HECT domain and RCC1-like domain-containing protein 2 (HERC2) Cyt-b5 domain, a Twist-related protein 1 (TWST1) HLH domain, an Homeobox protein Nkx-2.2 (NKX22) homeodomain, an Inhibitor of DNA binding 1 (ID3) HLH domain, and a Twist-related protein 2 (TWST2) HLH domain, and EED repressor domain; a Repressor Element Silencing Transcription Factor (REST) repression domain; a WRPW motif (SEQ ID NO: 76) of the hairy-related basic helix-loop-helix repressor proteins, the motif is known as a WRPW (SEQ ID NO: 76) repression domain; a DNA (cytosine-5)-methyltransferase 3B (DNMT3B) repression domain; and an HP1 alpha chromoshadow repression domain.

In some embodiments, the mifepristone-ITF comprises a peptide linker localized between the DNA binding domain and the transcriptional effector domain.

In some embodiments, the DNA binding domain is N-terminal to the transcriptional effector domain. In some embodiments, the mifepristone-ITF comprises, in the direction from N-terminus to C terminus: a DNA binding domain, a ligand binding domain (e.g., a progesterone receptor domain, and a transcriptional effector domain.

In some embodiments, the transcriptional effector domain is N-terminal to the DNA binding domain. In some embodiments, the mifepristone-ITF comprises, in the direction from N-terminus to C terminus: transcriptional effector domain, a ligand binding domain (e.g., a progesterone receptor domain, and a DNA binding domain.

When a mifepristone-ITF is contacted with a cognate ligand (i.e., mifepristone or a derivative thereof), the ITF translocates to the nucleus where it is capable of modulating transcription of a gene of interest.

In some embodiments, the gene of interest is operably linked to an ITF-responsive promoter. In some embodiments, the ITF-responsive promoter includes an ITF-binding domain sequence and a core promoter sequence.

Expression Systems for Producing an ITF Including Oligomerizable Antibody Domains and/or is Inducible by Caffeine, a Cannabinoid (e.g., a Phytocannabinoid Such as Cannabidiol), Nicotine ITF Formed by Oligomerizable Antibodies or Fragments Thereof

Also provided are expression systems for producing an inducible transcription factor (ITF) that includes a first monomer, the first monomer including a DNA binding domain a first antibody or fragment thereof that binds to a cognate ligand, and a second monomer, the second monomer including a transcriptional effector domain and a second antibody or fragment thereof that binds to the cognate ligand, wherein the first antibody or fragment thereof and the second antibody or fragment thereof are capable of oligomerizing (e.g., dimerizing) in the presence of the cognate ligand.

In some embodiments, the expression system for producing the ITF includes a first expression cassette comprising a first promoter an exogenous polynucleotide sequence encoding the first monomer, and a second expression cassette comprising a second promoter and an exogenous polynucleotide sequence encoding the second monomer.

In some embodiments, the first promoter and/or the second promoter is a constitutive promoter. In some embodiments, the first promoter and/or the second promoter is a constitutive promoter selected from: CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, and hUBIb.

In some embodiments, the first promoter and/or the second promoter is a regulatable promoter.

In some embodiments, the first promoter and/or the second promoter is a synthetic promoter.

Expression Systems for Producing an ITF that is Inducible by Caffeine, a Cannabinoid, or Nicotine

Also provided are expression systems for producing an inducible transcription factor (ITF) that is inducible by caffeine, a cannabinoid, or nicotine as described herein.

In some embodiments, the expression system for producing an inducible transcription factor (ITF) that is inducible by caffeine, a cannabinoid, or nicotine includes a first expression cassette comprising a first promoter an exogenous polynucleotide sequence encoding the first monomer as described herein, and a second expression cassette comprising a second promoter and an exogenous polynucleotide sequence encoding the second monomer as described herein.

In some embodiments, the first promoter and/or the second promoter is a constitutive promoter. In some embodiments, the first promoter and/or the second promoter is a constitutive promoter selected from: CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, and hUBIb.

In some embodiments, the first promoter and/or the second promoter is a regulatable promoter.

In some embodiments, the first promoter and/or the second promoter is a synthetic promoter.

Transcription Factors Including a ZF Domain with an Array of Six Zinc Finger Motifs

In some embodiments, the present disclosure provides an ITF that includes a first monomer, the first monomer including (i) a ZF domain comprising an array of six zinc finger motifs and (ii) a first ligand binding domain, and a second monomer, the second monomer including (i) a transcriptional effector domain and (ii) a second ligand binding domain, wherein the first and the second ligand binding domains are capable of oligomerizing (e.g., dimerizing) in the presence of the cognate ligand.

In some embodiments, the ITF including a ZF domain comprising an array of six zinc finger motifs is inducible by rapamycin or a derivative or analog thereof (referred to herein as a “rapamycin-inducible transcription factor” or “rapamycin-ITF”). The rapamycin-ITF includes a first monomer comprising a DNA binding domain and a first ligand binding domain, and a second monomer comprising a transcription effector domain and a second ligand binding domain. The DNA binding domain of a rapamycin-ITF comprises a zinc finger (ZFA) protein domain that is modular and comprises ten zinc finger arrays (ZFA). Upon binding to a cognate ligand selected from rapamycin, AP1903, AP20187, and FK1012, and derivatives and analogs thereof, the first monomer and the second monomer are oligomerizable. Ligand-induced oligomerization of the first and second monomers forms the rapamycin-ITF, which is capable of binding to a ZF protein binding site in a promoter operably linked to a gene of interest and modulating transcription of the gene of interest.

“Rapamycin or a derivative or analog thereof” refers to an immunosuppressive macrolide that blocks mammalian target of rapamycin (mTOR). Rapamycin and its derivatives and analogs have anti-proliferative activity, and have antifungal and/or anti-cancer activity. Examples of rapamycin derivatives or analogs include AP1903, AP20187, and FK1012.

In some embodiments, the rapamycin-ITF includes a first ligand binding domain and a second binding domain that each bind to rapamycin or a derivative or analog thereof. In some embodiments, the first monomer and the second monomer of the rapamycin-ITF are capable of forming a dimer upon binding to a ligand selected from rapamycin, AP1903, AP20187, and FK1012, or derivatives or analogs thereof.

In some embodiments, one of the first ligand binding domain or the second ligand binding domain of the rapamycin-ITF comprises FK506 binding protein (FKBP), and the other of the first or the second ligand binding domain comprises the FKBP-rapamycin binding (FRB) domain of the mammalian target of rapamycin (mTOR) kinase. FKBP (also known as FKB12) binds to rapamycin with high affinity (Kd=0.2 nM) and the FKBP-rapamycin complex is capable of associating with FRP, thus forming an FKBP-rapamycin-FRB complex. An exemplary FKBP is provided as SEQ ID NO: 60. An exemplary FRB is provided as SEQ ID NO: 61. In some embodiments, one of the first ligand binding domain or the second ligand binding domain comprises the amino acid sequence of SEQ ID NO: 60, and the other of the first or the second ligand binding domain comprises the amino acid sequence of SEQ ID NO: 61.

In some embodiments, the ZF protein domain of the rapamycin-ITF comprises the amino acid sequence of SEQ ID NO: 1.

In some embodiments, the transcriptional effector domain of the rapamycin-ITF is a transcriptional activator domain. In some embodiments, the transcriptional activator domain is selected from: a Herpes Simplex Virus Protein 16 (VP16) activation domain; an activation domain comprising four tandem copies of VP16; a VP64 activation domain; a p65 activation domain of NFκB; an Epstein-Barr virus R transactivator (Rta) activation domain; a tripartite activator comprising the VP64, the p65, and the Rta activation domains (VPR activation domain); and a histone acetyltransferase (HAT) core domain of the human E1A-associated protein p300 (p300 HAT core activation domain).

In some embodiments, the transcriptional effector domain of the rapamycin-ITF is a transcriptional repressor domain. In some embodiments, the transcriptional repressor domain is selected from: a Krüppel associated box (KRAB) repression domain; a truncated Krüppel associated box (KRAB) repression domain; a Histone Deacetylase 4 (HDAC4) repressor domain; a Scleraxis (SCX) HLH domain, an Inhibitor of DNA binding 1 (ID1) HLH domain, a HECT domain and RCC1-like domain-containing protein 2 (HERC2) Cyt-b5 domain, a Twist-related protein 1 (TWST1) HLH domain, an Homeobox protein Nkx-2.2 (NKX22) homeodomain, an Inhibitor of DNA binding 1 (ID3) HLH domain, and a Twist-related protein 2 (TWST2) HLH domain, and EED repressor domain; a Repressor Element Silencing Transcription Factor (REST) repression domain; a WRPW motif (SEQ ID NO: 76) of the hairy-related basic helix-loop-helix repressor proteins, the motif is known as a WRPW (SEQ ID NO: 76) repression domain; a DNA (cytosine-5)-methyltransferase 3B (DNMT3B) repression domain; and an HP1 alpha chromoshadow repression domain.

In some embodiments, the first monomer and/or the second monomer of the rapamycin-ITF includes a nuclear localization signal (NLS). In some embodiments, the NLS comprises the amino acid sequence of a sequence selected from SEQ ID NOs: 5-7. In some embodiments, the NLS comprises the amino acid sequence of SEQ ID NO: 5.

In some embodiments, the first monomer of rapamycin-ITF includes a nuclear localization signal (NLS). In some embodiments, the first monomer of the rapamycin-ITF includes a nuclear localization signal (NLS), and the second monomer of the ITF inducible by rapamycin or a derivative or analog thereof includes a nuclear localization signal (NLS).

In some embodiments, the second monomer of rapamycin-ITF includes a nuclear export signal (NES). In some embodiments, the NES comprises the amino acid sequence of a sequence selected from SEQ ID NOs: 8-17. In some embodiments, the NES comprises the amino acid sequence of SEQ ID NO: 8.

In some embodiments, the first monomer of the rapamycin-ITF includes a nuclear localization signal (NLS), and the second monomer of the ITF inducible by rapamycin or a derivative or analog thereof includes a nuclear export signal (NES).

In some embodiments, the first monomer of the rapamycin-ITF includes a peptide linker separating the DNA binding domain and the first ligand binding domain. In some embodiments, the DNA binding domain is N-terminal to the first ligand binding domain. In some embodiments, the first ligand binding domain is N-terminal to the DNA binding domain.

In some embodiments, the second monomer of the rapamycin-ITF includes a peptide linker separating the transcriptional effector domain and the second ligand binding domain. In some embodiments, the transcriptional effector domain is N-terminal to the second ligand binding domain. In some embodiments, the second ligand binding domain is N-terminal to the transcriptional effector domain.

When a rapamycin-ITF is formed by the ligand-induced oligomerization of the first and second monomers, the ITF is capable of modulating transcription of a gene of interest. The oligomerized product of the first and second monomers results in a functional ITF because it the quaternary protein structure of the oligomer includes both the DNA binding domain and the transcriptional effector domain.

Expression Systems for Producing an Oligomerizable ITF Including a ZF Domain with an Array of Six Zinc Finger Motifs

Also provided are expression systems for producing an ITF that includes a first monomer, the first monomer including (i) a ZF domain comprising an array of six zinc finger motifs and (ii) a first ligand binding domain, and a second monomer, the second monomer including (i) a transcriptional effector domain and (ii) a second ligand binding domain, wherein the first and the second ligand binding domains are capable of oligomerizing (e.g., dimerizing) in the presence of the cognate ligand. In some embodiments, the cognate ligand is selected from: rapamycin, AP1903, AP20187, FK1012, derivatives thereof, or analogs thereof and the expression system is for producing a rapamycin-inducible transcription factor (rapamycin-ITF) as described herein.

In some embodiments, the expression system includes a first expression cassette comprising a first promoter an exogenous polynucleotide sequence encoding the first monomer of the rapamycin-ITF as described herein, and a second expression cassette comprising a second promoter and an exogenous polynucleotide sequence encoding the second monomer of the rapamycin-ITF as described herein.

In some embodiments, the first promoter and/or the second promoter is a constitutive promoter. In some embodiments, the first promoter and/or the second promoter is a constitutive promoter selected from: CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, and hUBIb.

In some embodiments, the first promoter and or the second promoter is an inducible promoter.

In some embodiments, the first promoter and/or the second promoter is a synthetic promoter.

### Expression Systems for Producing a Mifepristone-ITF

Also provided are expression systems for producing a mifepristone-inducible transcription factor (mifepristone-ITF) as described herein.

In some embodiments, the expression system includes a first expression cassette comprising a first promoter and an exogenous polynucleotide sequence encoding a mifepristone-ITF as described herein.

In some embodiments, the first promoter and/or the second promoter is a constitutive promoter. In some embodiments, the first promoter and/or the second promoter is a constitutive promoter selected from: CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, and hUBIb.

In some embodiments, the first promoter and or the second promoter is an inducible promoter.

In some embodiments, the first promoter and/or the second promoter is a synthetic promoter.

In some embodiments, the expression system further includes a second expression cassette comprising an ITF-responsive promoter operably linked to a gene of interest, wherein the ITF-responsive promoter comprises a core promoter sequence and a sequence that binds to the DNA binding domain of the mifepristone-ITF. In some embodiments, the core promoter sequence comprises a minimal promoter.

### Expression Systems Including an ITF-Responsive Promoter

In some embodiments, the expression systems provided herein further include an expression cassette comprising an ITF-responsive promoter operably linked to a gene of interest, wherein the ITF-responsive promoter comprises a core promoter sequence and a sequence that binds to the DNA binding domain of the ITF.

In some embodiments, the ITFs described herein are capable of modulating transcription of a gene that is operably linked to an ITF-responsive promoter. “ITF-responsive promoter” as used herein refers to a synthetic regulatable promoter that is specifically recognized by an ITF as described herein, and that can be controlled by the presence of the cognate ligand of the ITF.

In some embodiments, the ITF-responsive promoter comprises a core promoter sequence and an ITF-binding domain that is specifically recognized by an inducible transcription factor (ITF) as described herein. “Core promoter sequence” as used herein refers to a portion of a promoter including a promoter sequence that interacts with RNA polymerase II and is sufficient to initiate transcription.

The ITF-binding domain binds to the DNA binding domain of the ITF and may include one or more zinc finger binding sites.

The binding domain may include one or more zinc finger binding sites. A zinc finger binding site is a polynucleotide sequence that is capable of binding to a zinc finger protein domain (e.g., the zinc finger protein domain of SEQ ID NO: 1). In some embodiments, the ZF protein domain of the ITF comprises an array of six zinc finger motifs, and ITF-binding domain of the ITF-responsive promoter comprises at least one binding site for the six zinc finger motifs. The binding domain can comprise 1, 2, 3, 4, 5, 6 7, 8, 9, 10, or more zinc finger binding sites. An exemplary zinc finger binding site comprises GGCGTAGCCGATGTCGCG (SEQ ID NO: 62). In some embodiments, the binding domain comprises one zinc finger binding site. In some embodiments, the binding domain comprises more than one zinc finger binding site. Zinc finger binding sites may be separated by a DNA linker. The DNA linker may be, in some embodiments, 5-40, 5-30, 10-40, 10-30 base pairs in length. In some embodiments, the binding domain comprises two zinc finger binding sites. In some embodiments, the binding domain comprises three zinc finger binding sites. In some embodiments, the binding domain comprises four zinc finger binding sites. An exemplary binding domain comprising zinc finger binding sites is shown in the sequence:

(SEQ ID NO: 63)
cgggtttcgtaacaatcgcatgaggattcgcaacgccttcGGCGTAGCCG
ATGTCGCGctcccgtctcagtaaaggtcGGCGTAGCCGATGTCGCGcaat
cggactgccttcgtacGGCGTAGCCGATGTCGCGcgtatcagtcgcctcg
gaacGGCGTAGCCGATGTCGCG.

The binding domain of SEQ ID NO: 63 includes four binding sites that each bind to a zinc finger protein domain of SEQ ID NO: 1, with each of the binding sites separated by a DNA linker.

In some embodiments, the core promoter sequence comprises a minimal promoter. In some embodiments, the core promoter sequence comprises a promoter selected from: minP, minCMV, YB_TATA, and minTK.

In some embodiments, the core promoter sequence comprises a core promoter sequence derived from a constitutive promoter sequence. In some embodiments, the core promoter sequence is derived from a constitutive promoter selected from CMV, EFS, SFFV, SV40, MND, PGK, UbC, EF1a, hCAGG, hACTb, heIF4A1, hGAPDH, hGRP78, hGRP94, hHSP70, hKINb, and hUBIb.

In some embodiments, the core promoter sequence includes TCTAGAGGGTATATAATGGGGGCCA (SEQ ID NO: 64).

An exemplary ITF-responsive promoter includes the sequence:

(SEQ ID NO: 65)
cgggtttcgtaacaatcgcatgaggattcgcaacgccttcGGCGTAGCCG
ATGTCGCGctcccgtctcagtaaaggtcGGCGTAGCCGATGTCGCGcaat
cggactgccttcgtacGGCGTAGCCGATGTCGCGcgtatcagtcgcctcg
gaacGGCGTAGCCGATGTCGCGcattcgtaagaggctcactctcccttac
acggagtggataACTAGTTCTAGAGGGTATATAATGGGGGCCA.

In some embodiments, the expression system for producing an ITF that is inducible by caffeine, a cannabinoid, or nicotine further includes a third expression cassette comprising an ITF-responsive promoter operably linked to a gene of interest, wherein the ITF-responsive promoter comprises a core promoter sequence and a sequence that binds to the DNA binding domain of the ITF.

In some embodiments, the expression system for producing a rapamycin-inducible transcription factor further includes a third expression cassette comprising an ITF-responsive promoter operably linked to a gene of interest, wherein the ITF-responsive promoter comprises a core promoter sequence and a sequence that binds to the DNA binding domain of the ITF.

In some embodiments, the expression system for producing a mifepristone-inducible transcription factor further includes second expression cassette comprising an ITF-responsive promoter operably linked to a gene of interest, wherein the ITF-responsive promoter comprises a core promoter sequence and a sequence that binds to the DNA binding domain of the ITF.

### Isolated Polynucleotide Molecules and Heterologous Constructs

Also provided herein are isolated polynucleotide molecules and heterologous constructs comprising one or more components of the expression systems as described herein.

“Isolated” nucleic acid molecule or polynucleotide refers to a nucleic acid molecule, such as DNA or RNA, which has been removed from its native environment. For example, a polynucleotide encoding an ITF as described herein contained in a heterologous construct is considered isolated. Further examples of an isolated polynucleotide include recombinant polynucleotides maintained in heterologous host cells or purified (partially or substantially) polynucleotides in solution. An isolated polynucleotide also includes a polynucleotide molecule contained in cells that ordinarily contain the polynucleotide molecule, but the polynucleotide molecule is present extrachromosomally or at a chromosomal location that is different from its natural chromosomal location.

Isolated polynucleotide molecules include, but are not limited to a cDNA polynucleotide, an RNA polynucleotide, an RNAi oligonucleotide (e.g., siRNAs, miRNAs, antisense oligonucleotides, shRNAs, etc.), an mRNA polynucleotide, a circular plasmid, a linear DNA fragment, a vector, a minicircle, a ssDNA, a bacterial artificial chromosome (BAC), and yeast artificial chromosome (YAC), and an oligonucleotide.

In some embodiments, the isolated polynucleotide molecule is selected from: a DNA, a cDNA, an RNA, an mRNA, and a naked plasmid (linear or circular).

By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98% or 99% identical to a nucleotide sequence of the present invention can be determined conventionally using known computer programs.

An “engineered polynucleotide” is a polynucleotide that does not occur in nature. It should be understood, however, that while an engineered polynucleotide as a whole is not naturally-occurring, it may include nucleotide sequences that occur in nature. In some embodiments, an engineered polynucleotide comprises nucleotide sequences from different organisms (e.g., from different species). For example, in some embodiments, an engineered polynucleotide includes a murine nucleotide sequence, a bacterial nucleotide sequence, a human nucleotide sequence, and/or a viral nucleotide sequence. The term “engineered polynucleotide” includes recombinant nucleic acids and synthetic nucleic acids. A “recombinant polynucleotide” refers to a molecule that is constructed by joining nucleotide molecules and, in some embodiments, can replicate in a live cell. A “synthetic polynucleotide” refers to a molecule that is amplified or chemically, or by other means, synthesized. Synthetic polynucleotides include those that are chemically modified, or otherwise modified, but can base pair with naturally-occurring nucleotide molecules. Modifications include, but are not limited to, one or more modified internucleotide linkages and non-natural nucleic acids. Modifications are described in further detail in U.S. Pat. No. 6,673,611 and U.S. Application Publication 2004/0019001 and, each of which is incorporated by reference in their entirety. Modified internucleotide linkages can be a phosphorodithioate or phosphorothioate linkage. Non-natural nucleic acids can be a locked nucleic acid (LNA), a peptide nucleic acid (PNA), glycol nucleic acid (GNA), a phosphorodiamidate morpholino oligomer (PMO or “morpholino”), and threose nucleic acid (TNA). Non-natural nucleic acids are described in further detail in International Application WO 1998/039352, U.S. Application Pub. No. 2013/0156849, and U.S. Pat. Nos. 6,670,461; 5,539,082; 5,185,444, each herein incorporated by reference in their entirety. Recombinant polynucleotides and synthetic polynucleotides also include those molecules that result from the replication of either of the foregoing. Engineered polynucleotides of the present disclosure may be encoded by a single molecule (e.g., included in the same plasmid or other vector) or by multiple different molecules (e.g., multiple different independently-replicating molecules).

Engineered polynucleotides of the present disclosure may be produced using standard molecular biology methods (see, e.g., Green and Sambrook, Molecular Cloning, A Laboratory Manual, 2012, Cold Spring Harbor Press). In some embodiments, engineered nucleic acid constructs are produced using GIBSON ASSEMBLY® Cloning (see, e.g., Gibson, D. G. et al. Nature Methods, 343-345, 2009; and Gibson, D. G. et al. Nature Methods, 901-903, 2010, each of which is incorporated by reference herein). GIBSON ASSEMBLY® typically uses three enzymatic activities in a single-tube reaction: 5′ exonuclease, the ′Y extension activity of a DNA polymerase and DNA ligase activity. The 5′ exonuclease activity chews back the 5′ end sequences and exposes the complementary sequence for annealing. The polymerase activity then fills in the gaps on the annealed regions. A DNA ligase then seals the nick and covalently links the DNA fragments together. The overlapping sequence of adjoining fragments is much longer than those used in Golden Gate Assembly, and therefore results in a higher percentage of correct assemblies. In some embodiments, engineered nucleic acid constructs are produced using IN-FUSION® cloning (Clontech).

In some embodiments, the polynucleotide molecules as described herein are included in a heterologous construct. The term “vector” or “expression vector” is synonymous with “heterologous construct” and refers to a polynucleotide molecule that is used to introduce and direct the expression of one or more genes that are operably associated with the construct in a target cell. The term includes the construct as a self-replicating nucleic acid structure as well as the vector incorporated into the genome of a host cell into which it has been introduced. In some embodiments, the heterologous construct comprises a lentiviral vector.

In some embodiments, provided herein is a first heterologous construct comprising a first expression cassette that comprises a polynucleotide molecule that encodes a first monomer of an ITF that is inducible by caffeine, a cannabinoid, or nicotine; and a second heterologous construct comprising a first expression cassette that comprises a polynucleotide molecule that encodes a second monomer of an ITF that is inducible by caffeine, a cannabinoid, or nicotine. In some embodiments, provided herein is a first heterologous construct comprising a first expression cassette that comprises a polynucleotide molecule that encodes a first monomer of a rapamycin-inducible transcription factor (rapamycin-ITF); and a second heterologous construct comprising a first expression cassette that comprises a polynucleotide molecule that encodes a second monomer of a rapamycin-inducible transcription factor (rapamycin-ITF).

In some embodiments, provided herein is a heterologous construct comprising an expression cassette that comprises a polynucleotide molecule that encodes a mifepristone-inducible transcription factor (mifepristone-ITF).

In some embodiments, engineered polynucleotides or heterologous constructs of the present disclosure are configured to produce multiple polypeptides. For example, polynucleotides may be configured to produce 2 different polypeptides (e.g., an ITF and a second polypeptide).

In some embodiments, engineered polynucleotides or heterologous constructs of the present disclosure are configured to produce multiple polypeptides. For example, polynucleotides may be configured to produce 2 different polypeptides (e.g., a first monomer and a second monomer of a rapamycin-ITF or an ITF that is inducible by caffeine, a cannabinoid, or nicotine). The polynucleotide molecule may be configured to produce the first monomer and the second monomer of the ITF, with both monomers under the control of the same promoter.

In some embodiments, engineered nucleic acids can be multicistronic, i.e., more than one separate polypeptide (e.g., a first monomer and a second monomer of a rapamycin-ITF or an ITF that is inducible by caffeine, a cannabinoid, nicotine, or mifepristone) can be produced from a single transcript. Engineered nucleic acids can be multicistronic through the use of various linkers, e.g., a polynucleotide sequence encoding a first exogenous polynucleotide can be linked to a nucleotide sequence encoding a second exogenous polynucleotide, such as in a first gene:linker:second gene 5′ to 3′ orientation. A linker polynucleotide sequence can encode one or more 2A ribosome skipping elements, such as T2A. Other 2A ribosome skipping elements include, but are not limited to, E2A, P2A, and F2A. 2A ribosome skipping elements allow production of separate polypeptides encoded by the first and second genes are produced during translation. A linker can encode a cleavable linker polypeptide sequence, such as a Furin cleavage site or a TEV cleavage site, wherein following expression the cleavable linker polypeptide is cleaved such that separate polypeptides encoded by the first and second genes are produced. A cleavable linker can include a polypeptide sequence, such as such a flexible linker (e.g., a Gly-Ser-Gly sequence), that further promotes cleavage.

A linker can encode an Internal Ribosome Entry Site (IRES), such that separate polypeptides encoded by the first and second genes are produced during translation. A linker can encode a splice acceptor, such as a viral splice acceptor.

A linker can be a combination of linkers, such as a Furin-2A linker that can produce separate polypeptides through 2A ribosome skipping followed by further cleavage of the Furin site to allow for complete removal of 2A residues. In some embodiments, a combination of linkers can include a Furin sequence, a flexible linker, and 2A linker. Accordingly, in some embodiments, the linker is a Furin-Gly-Ser-Gly-2A fusion polypeptide. In some embodiments, a linker is a Furin-Gly-Ser-Gly-T2A fusion polypeptide.

In general, a multicistronic system can use any number or combination of linkers, to express any number of genes or portions thereof (e.g., an engineered nucleic acid can encode a first, a second, and a third effector molecule, each separated by linkers such that separate polypeptides encoded by the first, second, and third effector molecules are produced).

“Linkers,” as used herein, can refer to peptide linkers that link a first polypeptide sequence and a second polypeptide sequence or can refer to the multicistronic linkers described above.

In some embodiments, provided herein is a heterologous construct comprising a multicistronic expression cassette that comprises a promoter; a first polynucleotide sequence that encodes a first monomer of an ITF that is inducible by caffeine, a cannabinoid, or nicotine; a second polynucleotide sequence that encodes a second monomer of an ITF that is inducible by caffeine, a cannabinoid, or nicotine; and a cleavable linker between the first polynucleotide and the second polynucleotide. In some embodiments, provided herein is a heterologous construct comprising a multicistronic expression cassette that comprises a promoter; a first polynucleotide sequence that encodes a first monomer of a rapamycin-inducible transcription factor; a second polynucleotide sequence that encodes a second monomer of a rapamycin-inducible transcription factor; and a cleavable linker between the first polynucleotide and the second polynucleotide.

In some embodiments, an engineered nucleic acid of the present disclosure comprises a post-transcriptional regulatory element (PRE). PREs can enhance gene expression via enabling tertiary RNA structure stability and 3′ end formation. Non-limiting examples of PREs include the Hepatitis B virus PRE (HPRE) and the Woodchuck Hepatitis Virus PRE (WPRE). In some embodiments, the post-transcriptional regulatory element is a Woodchuck Hepatitis Virus Posttranscriptional Regulatory Element (WPRE). In some embodiments, the WPRE comprises the alpha, beta, and gamma components of the WPRE element. In some embodiments, the WPRE comprises the alpha component of the WPRE element. Examples of WPRE sequences include SEQ ID NO: 66 and SEQ ID NO: 67.

### Engineered Cells

Also provided are cells, and methods of producing cells, that express the inducible transcription factors (ITFs) as described herein or comprise one or more expression systems or heterologous constructs of the present disclosure. These cells are referred to herein as “engineered cells.” These cells, which typically contain one or more engineered nucleic acids, do not occur in nature. In some embodiments, the cells are isolated cells that recombinantly express the one or more engineered polynucleotides. In some embodiments, the engineered polynucleotides are expressed from one or more vectors or a selected locus from the genome of the cell. In some embodiments, the cells are engineered to include a polynucleotide comprising a promoter operably linked to a nucleotide sequence.

An engineered cell of the present disclosure can comprise an engineered polynucleotide integrated into the cell's genome. An engineered cell can comprise an engineered polynucleotide capable of expression without integrating into the cell's genome, for example, engineered with a transient expression system such as a plasmid or mRNA.

In some embodiments, provided is an engineered cell comprising an ITF as described herein. In some embodiments, the cell further comprises a comprises an expression cassette comprising an ITF-responsive promoter as described herein, operably linked to an exogenous polynucleotide sequence encoding the gene of interest.

In some embodiments, provided is an engineered cell comprising an ITF that is inducible by caffeine or a derivative thereof (i.e., a caffeine-inducible transcription factor or “caffeine-ITF”) as described herein. In some embodiments, provided is an engineered cell comprising an expression system encoding the first and the second monomer of a caffeine-ITF as described herein.

In some embodiments, provided is an engineered cell comprising an ITF that is inducible by a cannabinoid (i.e., a cannabinoid-inducible transcription factor or “cannabinoid-ITF”) as described herein. In some embodiments, provided is an engineered cell comprising an expression system encoding the first and the second monomer of a cannabinoid-ITF as described herein.

In some embodiments, provided is an engineered cell comprising an ITF that is inducible by nicotine or a derivative thereof (i.e., nicotine-inducible transcription factor or “nicotine-ITF”) as described herein. In some embodiments, provided is an engineered cell comprising an expression system encoding the first and the second monomer of a nicotine-ITF as described herein.

In some embodiments, provided is an engineered cell comprising an ITF that is inducible by rapamycin or a derivative or an analog thereof (i.e., a rapamycin-inducible transcription factor or “rapamycin-ITF”) as described herein. In some embodiments, provided is an engineered cell comprising an expression system encoding the first and the second monomer of a rapamycin-ITF as described herein.

In some embodiments, provided is an engineered cell comprising an ITF that is inducible by mifepristone or a derivative thereof (i.e., a mifepristone-inducible transcription factor or “mifepristone-ITF”) as described herein. In some embodiments, provided is an engineered cell comprising an expression system encoding a mifepristone-ITF as described herein.

An engineered cell of the present disclosure can be a human cell. An engineered cell can be a human primary cell. An engineered primary cell can be any somatic cell. An engineered primary cell can be any stem cell. In some embodiments, the engineered cell is derived from the subject. In some embodiments, the engineered cell is allogeneic with reference to the subject.

An engineered cell of the present disclosure can be isolated from a subject, such as a subject known or suspected to have cancer. Cell isolation methods are known to those skilled in the art and include, but are not limited to, sorting techniques based on cell-surface marker expression, such as FACS sorting, positive isolation techniques, and negative isolation, magnetic isolation, and combinations thereof. An engineered cell can be allogenic with reference to the subject being administered a treatment. Allogenic modified cells can be HLA-matched to the subject being administered a treatment. An engineered cell can be a cultured cell, such as an ex vivo cultured cell. An engineered cell can be an ex vivo cultured cell, such as a primary cell isolated from a subject. Cultured cell can be cultured with one or more cytokines.

In some embodiments, an engineered cell of the present disclosure is selected from: a T cell (e.g., a CD8+ T cell, a CD4+ T cell, or a gamma-delta T cell), a cytotoxic T lymphocyte (CTL), a regulatory T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage (e.g., an M1 macrophage or an M2 macrophage), a monocyte, a dendritic cell, an erythrocyte, a platelet cell, a neuron, an oligodendrocyte, an astrocyte, a placode-derived cell, a Schwann cell, a cardiomyocyte, an endothelial cell, a nodal cell, a microglial cell, a hepatocyte, a cholangiocyte, a beta cell, a human embryonic stem cell (ESC), an ESC-derived cell, a pluripotent stem cell, a mesenchymal stromal cell (MSC), an induced pluripotent stem cell (iPSC), and an iPSC-derived cell.

In some embodiments, an engineered cell of the present disclosure is a T cell (e.g., a CD8+ T cell, a CD4+ T cell, or a gamma-delta T cell). In some embodiments, an engineered of the present disclosure is a cytotoxic T lymphocyte (CTL). In some embodiments, an engineered cell of the present disclosure is a regulatory T cell. In some embodiments, an engineered cell of the present disclosure is a Natural Killer T (NKT) cell. In some embodiments, an engineered cell of the present disclosure is a Natural Killer (NK) cell. In some embodiments, an engineered cell of the present disclosure is a B cell. In some embodiments, an engineered cell of the present disclosure is a tumor-infiltrating lymphocyte (TIL). In some embodiments, an engineered cell of the present disclosure is an innate lymphoid cell. In some embodiments, an engineered cell of the present disclosure is a mast cell. In some embodiments, an engineered cell of the present disclosure is an eosinophil. In some embodiments, an engineered cell of the present disclosure is a basophil. In some embodiments, an engineered cell of the present disclosure is a neutrophil. In some embodiments, an engineered cell of the present disclosure is a myeloid cell. In some embodiments, an engineered cell of the present disclosure is a macrophage e.g., an M1 macrophage or an M2 macrophage). In some embodiments, an engineered cell of the present disclosure is a monocyte. In some embodiments, an engineered or engineered cell of the present disclosure is a dendritic cell. In some embodiments, an engineered cell of the present disclosure is an erythrocyte. In some embodiments, an engineered cell of the present disclosure is a platelet cell. In some embodiments, a cell of the present disclosure is a neuron. In some embodiments, a cell of the present disclosure is a microglial cell. In some embodiments, a cell of the present disclosure is an oligodendrocyte. In some embodiments, a cell of the present disclosure is an astrocyte. In some embodiments, a cell of the present disclosure is a placode-derived cell. In some embodiments, an engineered cell of the present disclosure is a Schwann cell. In some embodiments, an engineered cell of the present disclosure is a cardiomyocyte. In some embodiments, an engineered cell of the present disclosure is an endothelial cell. In some embodiments, an engineered cell of the present disclosure is a nodal cell. In some embodiments, an engineered cell of the present disclosure is a microglial cell. In some embodiments, an engineered cell of the present disclosure is a hepatocyte. In some embodiments, an engineered cell of the present disclosure is a cholangiocyte. In some embodiments, an engineered cell of the present disclosure is a beta cell. In some embodiments, an engineered cell of the present disclosure is a human embryonic stem cell (ESC). In some embodiments, an engineered cell of the present disclosure is an ESC-derived cell. In some embodiments, an engineered cell of the present disclosure is a pluripotent stem cell. In some embodiments, an engineered cell of the present disclosure is a mesenchymal stromal cell (MSC). In some embodiments, an engineered cell of the present disclosure is an induced pluripotent stem cell (iPSC). In some embodiments, an engineered cell of the present disclosure is an iPSC-derived cell. In some embodiments, an engineered cell is autologous. In some embodiments, an engineered cell is allogeneic. In some embodiments, an engineered cell of the present disclosure is a CD34+ cell, a CD3+ cell, a CD8+ cell, a CD16+ cell, and/or a CD4+ cell.

In some embodiments, a cell of the present disclosure is a tumor cell selected from: an adenocarcinoma cell, a bladder tumor cell, a brain tumor cell (e.g., a glioma cell or a glioblastoma cell), a breast tumor cell, a cervical tumor cell, a colorectal tumor cell, an esophageal tumor cell, a glioma cell, a kidney tumor cell, a liver tumor cell, a lung tumor cell, a melanoma cell, a mesothelioma cell, an ovarian tumor cell, a pancreatic tumor cell, a prostate tumor cell, a skin tumor cell, a thyroid tumor cell, and a uterine tumor cell.

Also provided herein are methods that include culturing the engineered cells of the present disclosure. Methods of culturing the engineered cells described herein are known. One skilled in the art will recognize that culturing conditions will depend on the particular engineered cell of interest. One skilled in the art will recognize that culturing conditions will depend on the specific downstream use of the engineered cell, for example, specific culturing conditions for subsequent administration of the engineered cell to a subject.

### Methods of Engineering Cells

Also provided herein are compositions and methods for engineering cells with any polynucleotide molecule or construct as described herein.

In general, cells are engineered through introduction (i.e., delivery) of one or more polynucleotides of the present disclosure. Delivery methods include, but are not limited to, viral-mediated delivery, lipid-mediated transfection, nanoparticle delivery, electroporation, sonication, and cell membrane deformation by physical means. One skilled in the art will appreciate the choice of delivery method can depend on the specific cell type to be engineered.

### Viral-Mediated Delivery

Viral vector-based delivery platforms can be used to engineer cells. In general, a viral vector-based delivery platform engineers a cell through introducing (i.e., delivering) into a host cell. For example, a viral vector-based delivery platform can engineer a cell through introducing any of the engineered nucleic acids described herein. A viral vector-based delivery platform can be a nucleic acid, and as such, an engineered nucleic acid can also encompass an engineered virally derived nucleic acid. Such engineered virally derived nucleic acids can also be referred to as recombinant viruses or engineered viruses.

A viral vector-based delivery platform can encode more than one engineered nucleic acid, gene, or transgene within the same nucleic acid. For example, an engineered virally derived nucleic acid, e.g., a recombinant virus or an engineered virus, can encode one or more transgenes, including, but not limited to, any of the engineered nucleic acids described herein that encode one or more effector molecules. The one or more transgenes encoding the one or more effector molecules can be configured to express the one or more effector molecules. A viral vector-based delivery platform can encode one or more genes in addition to the one or more transgenes (e.g., transgenes encoding the one or more effector molecules), such as viral genes needed for viral infectivity and/or viral production (e.g., capsid proteins, envelope proteins, viral polymerases, viral transcriptases, etc.), referred to as cis-acting elements or genes.

A viral vector-based delivery platform can comprise more than one viral vector, such as separate viral vectors encoding the engineered nucleic acids, genes, or transgenes described herein, and referred to as trans-acting elements or genes. For example, a helper-dependent viral vector-based delivery platform can provide additional genes needed for viral infectivity and/or viral production on one or more additional separate vectors in addition to the vector encoding the one or more effector molecules. One viral vector can deliver more than one engineered nucleic acids, such as one vector that delivers engineered nucleic acids that are configured to produce two or more effector molecules. More than one viral vector can deliver more than one engineered nucleic acids, such as more than one vector that delivers one or more engineered nucleic acid configured to produce one or more effector molecules. The number of viral vectors used can depend on the packaging capacity of the above-mentioned viral vector-based vaccine platforms, and one skilled in the art can select the appropriate number of viral vectors.

In general, any of the viral vector-based systems can be used for the in vitro production of molecules, such as effector molecules, or used in vivo and ex vivo gene therapy procedures, e.g., for delivery of the engineered nucleic acids encoding one or more effector molecules. The selection of an appropriate viral vector-based system will depend on a variety of factors, such as cargo/payload size, immunogenicity of the viral system, target cell of interest, gene expression strength and timing, and other factors appreciated by one skilled in the art.

Viral vector-based delivery platforms can be RNA-based viruses or DNA-based viruses. Exemplary viral vector-based delivery platforms include, but are not limited to, a herpes simplex virus, an adenovirus, a measles virus, an influenza virus, a Indiana vesiculovirus, a Newcastle disease virus, a vaccinia virus, a poliovirus, a myxoma virus, a reovirus, a mumps virus, a Maraba virus, a rabies virus, a rotavirus, a hepatitis virus, a rubella virus, a dengue virus, a chikungunya virus, a respiratory syncytial virus, a lymphocytic choriomeningitis virus, a morbillivirus, a lentivirus, a replicating retrovirus, a rhabdovirus, a Seneca Valley virus, a sindbis virus, and any variant or derivative thereof. Other exemplary viral vector-based delivery platforms are described in the art, such as vaccinia, fowlpox, self-replicating alphavirus, marabavirus, adenovirus (See, e.g., Tatsis et al., Adenoviruses, Molecular Therapy (2004) 10, 616-629), or lentivirus, including but not limited to second, third or hybrid second/third generation lentivirus and recombinant lentivirus of any generation designed to target specific cell types or receptors (See, e.g., Hu et al., Immunization Delivered by Lentiviral Vectors for Cancer and Infectious Diseases, Immunol Rev. (2011) 239(1): 45-61, Sakuma et al., Lentiviral vectors: basic to translational, Biochem J. (2012) 443(3):603-18, Cooper et al., Rescue of splicing-mediated intron loss maximizes expression in lentiviral vectors containing the human ubiquitin C promoter, Nucl. Acids Res. (2015) 43 (1): 682-690, Zufferey et al., Self-Inactivating Lentivirus Vector for Safe and Efficient In vivo Gene Delivery, J. Virol. (1998) 72 (12): 9873-9880).

The sequences may be preceded with one or more sequences targeting a subcellular compartment. Upon introduction (i.e., delivery) into a host cell, infected cells (i.e., an engineered cell) can express the ITF as described herein. Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vectors useful for the introduction (i.e., delivery) of engineered nucleic acids, e.g., Salmonella typhi vectors, and the like will be apparent to those skilled in the art from the description herein.

The viral vector-based delivery platforms can be a virus that targets a tumor cell, herein referred to as an oncolytic virus. Examples of oncolytic viruses include, but are not limited to, an oncolytic herpes simplex virus, an oncolytic adenovirus, an oncolytic measles virus, an oncolytic influenza virus, an oncolytic Indiana vesiculovirus, an oncolytic Newcastle disease virus, an oncolytic vaccinia virus, an oncolytic poliovirus, an oncolytic myxoma virus, an oncolytic reovirus, an oncolytic mumps virus, an oncolytic Maraba virus, an oncolytic rabies virus, an oncolytic rotavirus, an oncolytic hepatitis virus, an oncolytic rubella virus, an oncolytic dengue virus, an oncolytic chikungunya virus, an oncolytic respiratory syncytial virus, an oncolytic lymphocytic choriomeningitis virus, an oncolytic morbillivirus, an oncolytic lentivirus, an oncolytic replicating retrovirus, an oncolytic rhabdovirus, an oncolytic Seneca Valley virus, an oncolytic sindbis virus, and any variant or derivative thereof. Any of the oncolytic viruses described herein can be a recombinant oncolytic virus comprising one more transgenes (e.g., an engineered nucleic acid) encoding one or more ITFs or ITF monomers. The transgenes encoding the one or more ITFs or ITF monomers can be configured to express the one or more ITFs or ITF monomers.

In some embodiments, the virus is selected from: a lentivirus, a retrovirus, an oncolytic virus, an adenovirus, an adeno-associated virus (AAV), and a virus-like particle (VLP).

The viral vector-based delivery platform can be retrovirus-based. In general, retroviral vectors are comprised of cis-acting long terminal repeats with packaging capacity for up to 6-10 kb of foreign sequence. The minimum cis-acting LTRs are sufficient for replication and packaging of the vectors, which are then used to integrate the one or more engineered nucleic acids (e.g., transgenes encoding the one or more ITFs or ITF monomers) into the target cell to provide permanent transgene expression. Retroviral-based delivery systems include, but are not limited to, those based upon murine leukemia, virus (MuL V), gibbon ape leukemia virus (GaLV), Simian Immunodeficiency virus (SIV), human immunodeficiency virus (HIV), and combinations thereof (see, e.g., Buchscher et al., J. Virol. 66:2731-2739 (1992); Johann et al, J. Virol. 66:1635-1640 (1992); Sommnerfelt et al., Virol. 176:58-59 (1990); Wilson et al, J. Virol. 63:2374-2378 (1989); Miller et al, J, Virol. 65:2220-2224 (1991); PCT/US94/05700). Other retroviral systems include the Phoenix retrovirus system.

The viral vector-based delivery platform can be lentivirus-based. In general, lentiviral vectors are retroviral vectors that are able to transduce or infect non-dividing cells and typically produce high viral titers. Lentiviral-based delivery platforms can be HIV-based, such as ViraPower systems (ThermoFisher) or pLenti systems (Cell Biolabs). Lentiviral-based delivery platforms can be SIV, or FIV-based. Other exemplary lentivirus-based delivery platforms are described in more detail in U.S. Pat. Nos. 7,311,907; 7,262,049; 7,250,299; 7,226,780; 7,220,578; 7,211,247; 7,160,721; 7,078,031; 7,070,993; 7,056,699; 6,955,919, each herein incorporated by reference for all purposes.

The viral vector-based delivery platform can be adenovirus-based. In general, adenoviral based vectors are capable of very high transduction efficiency in many cell types, do not require cell division, achieve high titer and levels of expression, and can be produced in large quantities in a relatively simple system. In general, adenoviruses can be used for transient expression of a transgene within an infected cell since adenoviruses do not typically integrate into a host's genome. Adenovirus-based delivery platforms are described in more detail in Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655, each herein incorporated by reference for all purposes. Other exemplary adenovirus-based delivery platforms are described in more detail in U.S. Pat. Nos. 5,585,362; 6,083,716, 7,371,570; 7,348,178; 7,323,177; 7,319,033; 7,318,919; and 7,306,793 and International Patent Application WO96/13597, each herein incorporated by reference for all purposes.

The viral vector-based delivery platform can be adeno-associated virus (AAV)-based. Adeno-associated virus (“AAV”) vectors may be used to transduce cells with engineered nucleic acids (e.g., any of the engineered nucleic acids described herein). AAV systems can be used for the in vitro production of ITFs or ITF monomers, or used in vivo and ex vivo gene therapy procedures, e.g., for in vivo delivery of the engineered nucleic acids encoding one or more ITFs or ITF monomers (see, e.g., West et al., Virology 160:38-47 (1987); U.S. Pat. Nos. 4,797,368; 5,436,146; 6,632,670; 6,642,051; 7,078,387; 7,314,912; 6,498,244; 7,906,111; US patent publications US 2003-0138772, US 2007/0036760, and US 2009/0197338; Gao, et al., J. Virol, 78(12):6381-6388 (June 2004); Gao, et al, Proc Natl Acad Sci USA, 100(10):6081-6086 (May 13, 2003); and International Patent applications WO 2010/138263 and WO 93/24641; Kotin, Human Gene Therapy 5:793-801 (1994); Muzyczka, J. Clin. Invest. 94:1351 (1994), each herein incorporated by reference for all purposes). Exemplary methods for constructing recombinant AAV vectors are described in more detail in U.S. Pat. No. 5,173,414; Tratschin et al, Mol. Cell. Biol. 5:3251-3260 (1985); Tratschin, et al, Mol. Cell, Biol. 4:2072-2081 (1984); Hermonat & Muzyczka, PNAS 81:64666470 (1984); and Samuiski et al, J. Virol. 63:03822-3828 (1989), each herein incorporated by reference for all purposes. In general, an AAV-based vector comprises a capsid protein having an amino acid sequence corresponding to any one of AAV1, AAV2, AAV3, AAV4, AAV5, AAV6, AAV7, AAV8, AAV9, AAV.Rh10, AAV11 and variants thereof.

The viral vector-based delivery platform can be a virus-like particle (VLP) platform. In general, VLPs are constructed by producing viral structural proteins and purifying resulting viral particles. Then, following purification, a cargo/payload (e.g., any of the engineered nucleic acids described herein) is encapsulated within the purified particle ex vivo. Accordingly, production of VLPs maintains separation of the nucleic acids encoding viral structural proteins and the nucleic acids encoding the cargo/payload. The viral structural proteins used in VLP production can be produced in a variety of expression systems, including mammalian, yeast, insect, bacterial, or in vivo translation expression systems. The purified viral particles can be denatured and reformed in the presence of the desired cargo to produce VLPs using methods known to those skilled in the art. Production of VLPs are described in more detail in Seow et al. (Mol Ther. 2009 May; 17(5): 767-777), herein incorporated by reference for all purposes.

The viral vector-based delivery platform can be engineered to target (i.e., infect) a range of cells, target a narrow subset of cells, or target a specific cell. In general, the envelope protein chosen for the viral vector-based delivery platform will determine the viral tropism. The virus used in the viral vector-based delivery platform can be pseudotyped to target a specific cell of interest. The viral vector-based delivery platform can be pantropic and infect a range of cells. For example, pantropic viral vector-based delivery platforms can include the VSV-G envelope. The viral vector-based delivery platform can be amphotropic and infect mammalian cells. Accordingly, one skilled in the art can select the appropriate tropism, pseudotype, and/or envelope protein for targeting a desired cell type.

### Lipid Structure Delivery Systems

Engineered nucleic acids of the present disclosure (e.g., a polynucleotide molecule encoding the first and second monomer of a rapamycin-ITF or an ITF that is inducible by caffeine, a cannabinoid, or nicotine, or a polynucleotide molecule encoding a mifepristone-ITF) can be introduced into a cell using a lipid-mediated delivery system. In general, a lipid-mediated delivery system uses a structure composed of an outer lipid membrane enveloping an internal compartment. Examples of lipid-based structures include, but are not limited to, a lipid-based nanoparticle, a liposome, a micelle, an exosome, a vesicle, an extracellular vesicle, a cell, or a tissue. Lipid structure delivery systems can deliver a cargo/payload (e.g., any of the engineered nucleic acids described herein) in vitro, in vivo, or ex vivo.

A lipid-based nanoparticle can include, but is not limited to, a unilamellar liposome, a multilamellar liposome, and a lipid preparation. As used herein, a “liposome” is a generic term encompassing in vitro preparations of lipid vehicles formed by enclosing a desired cargo, e.g., an engineered nucleic acid, such as any of the engineered nucleic acids described herein, within a lipid shell or a lipid aggregate. Liposomes may be characterized as having vesicular structures with a bilayer membrane, generally comprising a phospholipid, and an inner medium that generally comprises an aqueous composition. Liposomes include, but are not limited to, emulsions, foams, micelles, insoluble monolayers, liquid crystals, phospholipid dispersions, lamellar layers and the like. Liposomes can be unilamellar liposomes. Liposomes can be multilamellar liposomes. Liposomes can be multivesicular liposomes. Liposomes can be positively charged, negatively charged, or neutrally charged. In certain embodiments, the liposomes are neutral in charge. Liposomes can be formed from standard vesicle-forming lipids, which generally include neutral and negatively charged phospholipids and a sterol, such as cholesterol. The selection of lipids is generally guided by consideration of a desired purpose, e.g., criteria for in vivo delivery, such as liposome size, acid lability and stability of the liposomes in the blood stream. A variety of methods are available for preparing liposomes, as described in, e.g., Szoka et al., Ann. Rev. Biophys. Bioeng. 9; 467 (1980), U.S. Pat. Nos. 4,235,871, 4,501,728, 4,501,728, 4,837,028, and 5,019,369, each herein incorporated by reference for all purposes.

A multilamellar liposome is generated spontaneously when lipids comprising phospholipids are suspended in an excess of aqueous solution such that multiple lipid layers are separated by an aqueous medium. Water and dissolved solutes are entrapped in closed structures between the lipid bilayers following the lipid components undergoing self-rearrangement. A desired cargo (e.g., a polypeptide, a nucleic acid, a small molecule drug, an engineered nucleic acid, such as any of the engineered nucleic acids described herein, a viral vector, a viral-based delivery system, etc.) can be encapsulated in the aqueous interior of a liposome, attached to a liposome via a linking molecule that is associated with both the liposome and the polypeptide/nucleic acid, interspersed within the lipid bilayer of a liposome, entrapped in a liposome, complexed with a liposome, or otherwise associated with the liposome such that it can be delivered to a target entity. Lipophilic molecules or molecules with lipophilic regions may also dissolve in or associate with the lipid bilayer.

A liposome used according to the present embodiments can be made by different methods, as would be known to one of ordinary skill in the art. Preparations of liposomes are described in further detail in WO 2016/201323, International Applications PCT/US85/01161 and PCT/US89/05040, and U.S. Pat. Nos. 4,728,578, 4,728,575, 4,737,323, 4,533,254, 4,162,282, 4,310,505, and 4,921,706; each herein incorporated by reference for all purposes.

Liposomes can be cationic liposomes. Examples of cationic liposomes are described in more detail in U.S. Pat. Nos. 5,962,016; 5,030,453; 6,680,068, U.S. Application 2004/0208921, and International Patent Applications WO03/015757A1, WO04029213A2, and WO02/100435A1, each hereby incorporated by reference in their entirety.

Lipid-mediated gene delivery methods are described, for instance, in WO 96/18372; WO 93/24640; Mannino & Gould-Fogerite, BioTechniques 6(7): 682-691 (1988); U.S. Pat. No. 5,279,833 Rose U.S. Pat. No. 5,279,833; WO91/06309; and Felgner et al., Proc. Natl. Acad. Sci. USA 84: 7413-7414 (1987), each herein incorporated by reference for all purposes.

Exosomes are small membrane vesicles of endocytic origin that are released into the extracellular environment following fusion of multivesicular bodies with the plasma membrane. The size of exosomes ranges between 30 and 100 nm in diameter. Their surface consists of a lipid bilayer from the donor cell's cell membrane, and they contain cytosol from the cell that produced the exosome, and exhibit membrane proteins from the parental cell on the surface. Exosomes useful for the delivery of nucleic acids are known to those skilled in the art, e.g., the exosomes described in more detail in U.S. Pat. No. 9,889,210, herein incorporated by reference for all purposes.

As used herein, the term “extracellular vesicle” or “EV” refers to a cell-derived vesicle comprising a membrane that encloses an internal space. In general, extracellular vesicles comprise all membrane-bound vesicles that have a smaller diameter than the cell from which they are derived. Generally extracellular vesicles range in diameter from 20 nm to 1000 nm, and can comprise various macromolecular cargo either within the internal space, displayed on the external surface of the extracellular vesicle, and/or spanning the membrane. The cargo can comprise nucleic acids (e.g., any of the engineered nucleic acids described herein), proteins, carbohydrates, lipids, small molecules, and/or combinations thereof. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, and/or cultured cells.

As used herein the term “exosome” refers to a cell-derived small (between 20-300 nm in diameter, more preferably 40-200 nm in diameter) vesicle comprising a membrane that encloses an internal space, and which is generated from the cell by direct plasma membrane budding or by fusion of the late endosome with the plasma membrane. The exosome comprises lipid or fatty acid and polypeptide and optionally comprises a payload (e.g., a therapeutic agent), a receiver (e.g., a targeting moiety), a polynucleotide (e.g., a nucleic acid, RNA, or DNA, such as any of the engineered nucleic acids described herein), a sugar (e.g., a simple sugar, polysaccharide, or glycan) or other molecules. The exosome can be derived from a producer cell, and isolated from the producer cell based on its size, density, biochemical parameters, or a combination thereof. An exosome is a species of extracellular vesicle. Generally, exosome production/biogenesis does not result in the destruction of the producer cell. Exosomes and preparation of exosomes are described in further detail in WO 2016/201323, which is hereby incorporated by reference in its entirety.

As used herein, the term “nanovesicle” (also referred to as a “microvesicle”) refers to a cell-derived small (between 20-250 nm in diameter, more preferably 30-150 nm in diameter) vesicle comprising a membrane that encloses an internal space, and which is generated from the cell by direct or indirect manipulation such that said nanovesicle would not be produced by said producer cell without said manipulation. In general, a nanovesicle is a sub-species of an extracellular vesicle. Appropriate manipulations of the producer cell include but are not limited to serial extrusion, treatment with alkaline solutions, sonication, or combinations thereof. The production of nanovesicles may, in some instances, result in the destruction of said producer cell. Preferably, populations of nanovesicles are substantially free of vesicles that are derived from producer cells by way of direct budding from the plasma membrane or fusion of the late endosome with the plasma membrane. The nanovesicle comprises lipid or fatty acid and polypeptide, and optionally comprises a payload (e.g., a therapeutic agent), a receiver (e.g., a targeting moiety), a polynucleotide (e.g., a nucleic acid, RNA, or DNA, such as any of the engineered nucleic acids described herein), a sugar (e.g., a simple sugar, polysaccharide, or glycan) or other molecules. The nanovesicle, once it is derived from a producer cell according to said manipulation, may be isolated from the producer cell based on its size, density, biochemical parameters, or a combination thereof.

Lipid nanoparticles (LNPs), in general, are synthetic lipid structures that rely on the amphiphilic nature of lipids to form membranes and vesicle like structures (Riley 2017). In general, these vesicles deliver cargo/payloads, such as any of the engineered nucleic acids or viral systems described herein, by absorbing into the membrane of target cells and releasing the cargo into the cytosol. Lipids used in LNP formation can be cationic, anionic, or neutral. The lipids can be synthetic or naturally derived, and in some instances biodegradable. Lipids can include fats, cholesterol, phospholipids, lipid conjugates including, but not limited to, polyethyleneglycol (PEG) conjugates (PEGylated lipids), waxes, oils, glycerides, and fat soluble vitamins. Lipid compositions generally include defined mixtures of materials, such as the cationic, neutral, anionic, and amphipathic lipids. In some instances, specific lipids are included to prevent LNP aggregation, prevent lipid oxidation, or provide functional chemical groups that facilitate attachment of additional moieties. Lipid composition can influence overall LNP size and stability. In an example, the lipid composition comprises dilinoleylmethyl-4-dimethylaminobutyrate (MC3) or MC3-like molecules. MC3 and MC3-like lipid compositions can be formulated to include one or more other lipids, such as a PEG or PEG-conjugated lipid, a sterol, or neutral lipids. In addition, LNPs can be further engineered or functionalized to facilitate targeting of specific cell types. Another consideration in LNP design is the balance between targeting efficiency and cytotoxicity.

Micelles, in general, are spherical synthetic lipid structures that are formed using single-chain lipids, where the single-chain lipid's hydrophilic head forms an outer layer or membrane and the single-chain lipid's hydrophobic tails form the micelle center. Micelles typically refer to lipid structures only containing a lipid mono-layer. Micelles are described in more detail in Quader et al. (Mol Ther. 2017 Jul. 5; 25(7): 1501-1513), herein incorporated by reference for all purposes.

Nucleic-acid vectors, such as expression vectors, exposed directly to serum can have several undesirable consequences, including degradation of the nucleic acid by serum nucleases or off-target stimulation of the immune system by the free nucleic acids. Similarly, viral delivery systems exposed directly to serum can trigger an undesired immune response and/or neutralization of the viral delivery system. Therefore, encapsulation of an engineered nucleic acid and/or viral delivery system can be used to avoid degradation, while also avoiding potential off-target affects. In certain examples, an engineered nucleic acid and/or viral delivery system is fully encapsulated within the delivery vehicle, such as within the aqueous interior of an LNP. Encapsulation of an engineered nucleic acid and/or viral delivery system within an LNP can be carried out by techniques well-known to those skilled in the art, such as microfluidic mixing and droplet generation carried out on a microfluidic droplet generating device. Such devices include, but are not limited to, standard T-junction devices or flow-focusing devices. In an example, the desired lipid formulation, such as MC3 or MC3-like containing compositions, is provided to the droplet generating device in parallel with an engineered nucleic acid or viral delivery system and any other desired agents, such that the delivery vector and desired agents are fully encapsulated within the interior of the MC3 or MC3-like based LNP. In an example, the droplet generating device can control the size range and size distribution of the LNPs produced. For example, the LNP can have a size ranging from 1 to 1000 nanometers in diameter, e.g., 1, 10, 50, 100, 500, or 1000 nanometers. Following droplet generation, the delivery vehicles encapsulating the cargo/payload (e.g., an engineered nucleic acid and/or viral delivery system) can be further treated or engineered to prepare them for administration.

### Nanoparticle Delivery

Nanomaterials can be used to deliver engineered nucleic acids (e.g., a polynucleotide molecule encoding a chimeric polypeptide). Nanomaterial vehicles, importantly, can be made of non-immunogenic materials and generally avoid eliciting immunity to the delivery vector itself. These materials can include, but are not limited to, lipids (as previously described), inorganic nanomaterials, and other polymeric materials. Nanomaterial particles are described in more detail in Riley et al. (Recent Advances in Nanomaterials for Gene Delivery—A Review. Nanomaterials 2017, 7(5), 94), herein incorporated by reference for all purposes.

### Genomic Editing Systems

Genomic editing systems can be used to engineer a host genome to encode an engineered nucleic acid, such as a polynucleotide molecule encoding an ITF or a monomer of an ITF of the present disclosure. In general, a “genomic editing system” refers to any system for integrating an exogenous gene into a host cell's genome. Genomic editing systems include, but are not limited to, a transposon system, a nuclease genomic editing system, and a viral vector-based delivery platform.

A transposon system can be used to integrate an engineered nucleic acid, such as an engineered nucleic acid of the present disclosure, into a host genome. Transposons generally comprise terminal inverted repeats (TIR) that flank a cargo/payload nucleic acid and a transposase. The transposon system can provide the transposon in cis or in trans with the TIR-flanked cargo. A transposon system can be a retrotransposon system or a DNA transposon system. In general, transposon systems integrate a cargo/payload (e.g., an engineered nucleic acid) randomly into a host genome. Examples of transposon systems include systems using a transposon of the Tc1/mariner transposon superfamily, such as a Sleeping Beauty transposon system, described in more detail in Hudecek et al. (Crit Rev Biochem Mol Biol. 2017 August; 52(4):355-380), and U.S. Pat. Nos. 6,489,458, 6,613,752 and 7,985,739, each of which is herein incorporated by reference for all purposes. Another example of a transposon system includes a PiggyBac transposon system, described in more detail in U.S. Pat. Nos. 6,218,185 and 6,962,810, each of which is herein incorporated by reference for all purposes.

A nuclease genomic editing system can be used to engineer a host genome to encode an engineered nucleic acid, such as an isolated polynucleotide or heterologous construct of the present disclosure. Without wishing to be bound by theory, in general, the nuclease-mediated gene editing systems used to introduce an exogenous gene take advantage of a cell's natural DNA repair mechanisms, particularly homologous recombination (HR) repair pathways. Briefly, following an insult to genomic DNA (typically a double-stranded break), a cell can resolve the insult by using another DNA source that has identical, or substantially identical, sequences at both its 5′ and 3′ ends as a template during DNA synthesis to repair the lesion. In a natural context, HDR can use the other chromosome present in a cell as a template. In gene editing systems, exogenous polynucleotides are introduced into the cell to be used as a homologous recombination template (HRT or HR template). In general, any additional exogenous sequence not originally found in the chromosome with the lesion that is included between the 5′ and 3′ complimentary ends within the HRT (e.g., a gene or a portion of a gene) can be incorporated (i.e., “integrated”) into the given genomic locus during templated HDR. Thus, a typical HR template for a given genomic locus has a nucleotide sequence identical to a first region of an endogenous genomic target locus, a nucleotide sequence identical to a second region of the endogenous genomic target locus, and a nucleotide sequence encoding a cargo/payload nucleic acid (e.g., any of the engineered nucleic acids described herein, such as any of the engineered nucleic acids encoding one or more ITFs or ITF monomers).

In some examples, a HR template can be linear. Examples of linear HR templates include, but are not limited to, a linearized plasmid vector, a ssDNA, a synthesized DNA, and a PCR amplified DNA. In particular examples, a HR template can be circular, such as a plasmid. A circular template can include a supercoiled template.

The identical, or substantially identical, sequences found at the 5′ and 3′ ends of the HR template, with respect to the exogenous sequence to be introduced, are generally referred to as arms (HR arms). HR arms can be identical to regions of the endogenous genomic target locus (i.e., 100% identical). HR arms in some examples can be substantially identical to regions of the endogenous genomic target locus. While substantially identical HR arms can be used, it can be advantageous for HR arms to be identical as the efficiency of the HDR pathway may be impacted by HR arms having less than 100% identity.

Each HR arm, i.e., the 5′ and 3′ HR arms, can be the same size or different sizes. Each HR arm can each be greater than or equal to 50, 100, 200, 300, 400, or 500 bases in length. Although HR arms can, in general, be of any length, practical considerations, such as the impact of HR arm length and overall template size on overall editing efficiency, can also be taken into account. An HR arms can be identical, or substantially identical to, regions of an endogenous genomic target locus immediately adjacent to a cleavage site. Each HR arms can be identical to, or substantially identical to, regions of an endogenous genomic target locus immediately adjacent to a cleavage site. Each HR arms can be identical, or substantially identical to, regions of an endogenous genomic target locus within a certain distance of a cleavage site, such as 1 base-pair, less than or equal to 10 base-pairs, less than or equal to 50 base-pairs, or less than or equal to 100 base-pairs of each other.

A nuclease genomic editing system can use a variety of nucleases to cut a target genomic locus, including, but not limited to, a Clustered Regularly Interspaced Short Palindromic Repeats (CRISPR) family nuclease or derivative thereof, a Transcription activator-like effector nuclease (TALEN) or derivative thereof, a zinc-finger nuclease (ZFN) or derivative thereof, and a homing endonuclease (HE) or derivative thereof.

A CRISPR-mediated gene editing system can be used to engineer a host genome to encode an engineered nucleic acid, such as an engineered nucleic acid encoding one or more of the ITFs or ITF monomers described herein. CRISPR systems are described in more detail in M. Adli (“The CRISPR tool kit for genome editing and beyond” Nature Communications; volume 9 (2018), Article number: 1911), herein incorporated by reference for all that it teaches. In general, a CRISPR-mediated gene editing system comprises a CRISPR-associated (Cas) nuclease and an RNA(s) that directs cleavage to a particular target sequence. An exemplary CRISPR-mediated gene editing system is the CRISPR/Cas9 systems comprised of a Cas9 nuclease and an RNA(s) that has a CRISPR RNA (crRNA) domain and a trans-activating CRISPR (tracrRNA) domain. The crRNA typically has two RNA domains: a guide RNA sequence (gRNA) that directs specificity through base-pair hybridization to a target sequence (“a defined nucleotide sequence”), e.g., a genomic sequence; and an RNA domain that hybridizes to a tracrRNA. A tracrRNA can interact with and thereby promote recruitment of a nuclease (e.g., Cas9) to a genomic locus. The crRNA and tracrRNA polynucleotides can be separate polynucleotides. The crRNA and tracrRNA polynucleotides can be a single polynucleotide, also referred to as a single guide RNA (sgRNA). While the Cas9 system is illustrated here, other CRISPR systems can be used, such as the Cpf1 system. Nucleases can include derivatives thereof, such as Cas9 functional mutants, e.g., a Cas9 “nickase” mutant that in general mediates cleavage of only a single strand of a defined nucleotide sequence as opposed to a complete double-stranded break typically produced by Cas9 enzymes.

In general, the components of a CRISPR system interact with each other to form a Ribonucleoprotein (RNP) complex to mediate sequence specific cleavage. In some CRISPR systems, each component can be separately produced and used to form the RNP complex. In some CRISPR systems, each component can be separately produced in vitro and contacted (i.e., “complexed”) with each other in vitro to form the RNP complex. The in vitro produced RNP can then be introduced (i.e., “delivered”) into a cell's cytosol and/or nucleus, e.g., a T cell's cytosol and/or nucleus. The in vitro produced RNP complexes can be delivered to a cell by a variety of means including, but not limited to, electroporation, lipid-mediated transfection, cell membrane deformation by physical means, lipid nanoparticles (LNP), virus like particles (VLP), and sonication. In a particular example, in vitro produced RNP complexes can be delivered to a cell using a Nucleofactor/Nucleofection® electroporation-based delivery system (Lonza®). Other electroporation systems include, but are not limited to, MaxCyte electroporation systems, Miltenyi CliniMACS electroporation systems, Neon electroporation systems, and BTX electroporation systems. CRISPR nucleases, e.g., Cas9, can be produced in vitro (i.e., synthesized and purified) using a variety of protein production techniques known to those skilled in the art. CRISPR system RNAs, e.g., an sgRNA, can be produced in vitro (i.e., synthesized and purified) using a variety of RNA production techniques known to those skilled in the art, such as in vitro transcription or chemical synthesis.

An in vitro produced RNP complex can be complexed at different ratios of nuclease to gRNA. An in vitro produced RNP complex can be also be used at different amounts in a CRISPR-mediated editing system. For example, depending on the number of cells desired to be edited, the total RNP amount added can be adjusted, such as a reduction in the amount of RNP complex added when editing a large number of cells in a reaction.

In some CRISPR systems, each component (e.g., Cas9 and an sgRNA) can be separately encoded by a polynucleotide with each polynucleotide introduced into a cell together or separately. In some CRISPR systems, each component can be encoded by a single polynucleotide (i.e., a multi-promoter or multicistronic vector, see description of exemplary multicistronic systems below) and introduced into a cell. Following expression of each polynucleotide encoded CRISPR component within a cell (e.g., translation of a nuclease and transcription of CRISPR RNAs), an RNP complex can form within the cell and can then direct site-specific cleavage.

Some RNPs can be engineered to have moieties that promote delivery of the RNP into the nucleus. For example, a Cas9 nuclease can have a nuclear localization signal (NLS) domain such that if a Cas9 RNP complex is delivered into a cell's cytosol or following translation of Cas9 and subsequent RNP formation, the NLS can promote further trafficking of a Cas9 RNP into the nucleus.

The cells described herein can be engineered using non-viral methods, e.g., the nuclease and/or CRISPR mediated gene editing systems described herein can be delivered to a cell using non-viral methods. The cells described herein can be engineered using viral methods, e.g., the nuclease and/or CRISPR mediated gene editing systems described herein can be delivered to a cell using viral methods such as adenoviral, retroviral, lentiviral, or any of the other viral-based delivery methods described herein.

In some CRISPR systems, more than one CRISPR composition can be provided such that each separately target the same gene or general genomic locus at more than target nucleotide sequence. For example, two separate CRISPR compositions can be provided to direct cleavage at two different target nucleotide sequences within a certain distance of each other. In some CRISPR systems, more than one CRISPR composition can be provided such that each separately target opposite strands of the same gene or general genomic locus. For example, two separate CRISPR “nickase” compositions can be provided to direct cleavage at the same gene or general genomic locus at opposite strands.

In general, the features of a CRISPR-mediated editing system described herein can apply to other nuclease-based genomic editing systems. TALEN is an engineered site-specific nuclease, which is composed of the DNA-binding domain of TALE (transcription activator-like effectors) and the catalytic domain of restriction endonuclease Fokl. By changing the amino acids present in the highly variable residue region of the monomers of the DNA binding domain, different artificial TALENs can be created to target various nucleotides sequences. The DNA binding domain subsequently directs the nuclease to the target sequences and creates a double-stranded break. TALEN-based systems are described in more detail in U.S. Ser. No. 12/965,590; U.S. Pat. Nos. 8,450,471; 8,440,431; 8,440,432; 10,172,880; and U.S. Ser. No. 13/738,381, all of which are incorporated by reference herein in their entirety. ZFN-based editing systems are described in more detail in U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties for all purposes.

### Other Engineering Delivery Systems

Various additional means to introduce engineered nucleic acids (e.g., a polynucleotide molecule encoding an ITF or a monomer of an ITF as described herein) into a cell or other target recipient entity, such as any of the lipid structures described herein.

Electroporation can used to deliver polynucleotides to recipient entities. Electroporation is a method of internalizing a cargo/payload into a target cell or entity's interior compartment through applying an electrical field to transiently permeabilize the outer membrane or shell of the target cell or entity. In general, the method involves placing cells or target entities between two electrodes in a solution containing a cargo of interest (e.g., any of the engineered nucleic acids described herein). The lipid membrane of the cells is then disrupted, i.e., permeabilized, by applying a transient set voltage that allows the cargo to enter the interior of the entity, such as the cytoplasm of the cell. In the example of cells, at least some, if not a majority, of the cells remain viable. Cells and other entities can be electroporated in vitro, in vivo, or ex vivo. Electroporation conditions (e.g., number of cells, concentration of cargo, recovery conditions, voltage, time, capacitance, pulse type, pulse length, volume, cuvette length, electroporation solution composition, etc.) vary depending on several factors including, but not limited to, the type of cell or other recipient entity, the cargo to be delivered, the efficiency of internalization desired, and the viability desired. Optimization of such criteria are within the scope of those skilled in the art. A variety devices and protocols can be used for electroporation. Examples include, but are not limited to, Neon® Transfection System, MaxCyte® Flow Electroporation™, Lonza® Nucleofector™ systems, and Bio-Rad® electroporation systems.

Other means for introducing engineered nucleic acids (e.g., a polynucleotide molecule encoding an ITF or a monomer of an ITF as described herein) into a cell or other target recipient entity include, but are not limited to, sonication, gene gun, hydrodynamic injection, and cell membrane deformation by physical means.

Compositions and methods for delivering engineered mRNAs in vivo, such as naked plasmids or mRNA, are described in detail in Kowalski et al. (Mol Ther. 2019 Apr. 10; 27(4): 710-728) and Kaczmarek et al. (Genome Med. 2017; 9: 60), each herein incorporated by reference for all purposes.

### Genetic Switches

Also provided are genetic switches including (i) an isolated cell comprising an ITF as described herein, and (ii) a cognate ligand. In some aspects, the isolated cell further comprises a gene operably linked to an ITF-responsive promoter.

In some embodiments, the genetic switch includes (i) an isolated cell comprising a caffeine-inducible transcription factor and (ii) caffeine.

In some embodiments, the genetic switch includes (i) an isolated cell comprising a cannabinoid-inducible transcription factor and (ii) a cannabinoid. In some embodiments, the cannabinoid is a phytocannabinoid. In some embodiments, the cannabinoid is cannabidiol.

In some embodiments, the genetic switch includes (i) an isolated cell comprising a nicotine-inducible transcription factor and (ii) nicotine.

In some embodiments, the genetic switch includes (i) an isolated cell comprising a rapamycin-inducible transcription factor and (ii) rapamycin or an analog or derivative thereof.

In some embodiments, the genetic switch includes (i) an isolated cell comprising a mifepristone-inducible transcription factor and (ii) mifepristone or derivative thereof.

### Methods of Use

Methods of using the ITFs as described herein are also encompassed by this disclosure. In some aspects, the methods include modulating transcription of a gene of interest.

In some aspects, provided herein is a method of modulating transcription, the method including: contacting an isolated cell expressing a first monomer and a second monomer of an inducible transcription factor that is inducible by caffeine, a cannabinoid, or nicotine as described herein with a cognate ligand selected from: caffeine, a cannabinoid, and nicotine, to induce formation of the inducible transcription factor (ITF); and culturing the isolated cell for a time period sufficient to allow the activated ITF to modulate transcription of a gene operably linked to an ITF-responsive promoter.

In some aspects, provided herein is a method of modulating transcription, the method including: contacting an isolated cell expressing a first monomer and a second monomer of a rapamycin-inducible transcription factor as described herein with a cognate ligand comprising rapamycin or a derivative or analog thereof, to induce formation of the inducible transcription factor (ITF); and culturing the isolated cell for a time period sufficient to allow the activated ITF to modulate transcription of a gene operably linked to an ITF-responsive promoter.

In some aspects, provided herein is a method of modulating transcription, the method including: contacting an isolated cell expressing a mifepristone-inducible transcription factor with mifepristone to induce nuclear localization of the inducible transcription factor (ITF), and culturing the isolated cell for a time period sufficient to allow the ITF to modulate transcription of a gene operably linked to an ITF-responsive promoter.

In some embodiments, the method of modulating transcription is a method of activating transcription. In such embodiments, the transcriptional effector domain of the inducible transcription factor may be a transcriptional activation domain.

In some embodiments, the method of modulating transcription is a method of repressing transcription. In such embodiments, the transcriptional effector domain of the inducible transcription factor may be a transcriptional repressor domain.

In some aspects, methods for treatment of diseases are also encompassed by this disclosure. Said methods include administering to a subject a therapeutically effective amount of an engineered polynucleotide encoding an inducible transcription factor or an isolated cell comprising a polynucleotide molecule encoding an inducible transcription factor as described above. In some aspects, provided herein are methods of treating a subject in need thereof, the method comprising administering a therapeutically effective dose of any of the engineered cells, isolated cells, or compositions disclosed herein.

### In Vivo Methods

In some aspects, methods provided herein also include modulating transcription of a gene of interest in vivo, e.g., by delivering to a subject (i) a cell engineered to produce an ITF as described herein and (ii) a cognate ligand (e.g., caffeine, a cannabinoid, nicotine, rapamycin, or mifepristone).

In some embodiments, the subject is a human or animal, and contacting the transformed cell with the cognate ligand comprises administering a pharmacological dose of the cognate ligand to the human or animal.

In some aspects, methods provided herein also include delivering a composition in vivo capable of producing the engineered cells described herein, e.g., capable of delivering any of the expression systems or polynucleotide molecules described herein to a cell in vivo. Such compositions include any of the viral-mediated delivery platforms, any of the lipid structure delivery systems, any of the nanoparticle delivery systems, any of the genomic editing systems, or any of the other engineering delivery systems described herein capable of engineering a cell in vivo.

The methods provided herein also include delivering a composition in vivo capable of producing any of the inducible transcription factors (and in some embodiments, a gene regulated by the inducible transcription factor) as described herein. Compositions capable of in vivo production of the inducible transcription factors (and in some embodiments, a gene regulated by the inducible transcription factor) include, but are not limited to, any of the engineered nucleic acids described herein. Compositions capable of in vivo production of inducible transcription factors (and in some embodiments, a gene regulated by the inducible transcription factor) can be a naked mRNA or a naked plasmid.

### Pharmaceutical Compositions

The inducible transcription factors, expression systems, and cells of the present disclosure can be formulated in pharmaceutical compositions. These compositions can comprise, in addition to one or more of the engineered nucleic acids or engineered cells, a pharmaceutically acceptable excipient, carrier, buffer, stabilizer or other materials well known to those skilled in the art. Such materials should be non-toxic and should not interfere with the efficacy of the active ingredient. The precise nature of the carrier or other material can depend on the route of administration, e.g., oral, intravenous, cutaneous or subcutaneous, nasal, intramuscular, intraperitoneal routes.

Whether it is a cell, polypeptide, nucleic acid, small molecule or other pharmaceutically useful compound according to the present disclosure that is to be given to an individual, administration is preferably in a “therapeutically effective amount” or “prophylactically effective amount” (as the case can be, although prophylaxis can be considered therapy), this being sufficient to show benefit to the individual. The actual amount administered, and rate and time-course of administration, will depend on the nature and severity of the disease being treated. Prescription of treatment, e.g., decisions on dosage, etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

A composition can be administered alone or in combination with other treatments, either simultaneously or sequentially dependent upon the condition to be treated.

### Enumerated Embodiments

1. An engineered cell comprising an inducible transcription factor (ITF), wherein the ITF comprises:
   a first monomer, wherein the first monomer comprises a DNA binding domain and a first ligand binding domain; and
   a second monomer, wherein the second monomer comprises a transcriptional effector domain and a second ligand binding domain,
wherein each monomer is oligomerizable via a cognate ligand that binds to each ligand binding domain,
wherein ligand-induced oligomerization forms the ITF, and
wherein the cognate ligand is selected from the group consisting of: caffeine, a cannabidiol (CBD), a phytocannabinoid, and nicotine.
2. The engineered cell of embodiment 1, wherein the DNA binding domain comprises a zinc finger (ZF) protein domain.
3. The engineered cell of embodiment 2, wherein the ZF protein domain is modular in design and is composed of one or more zinc finger arrays (ZFA).
4. The engineered cell of embodiment 3, wherein the ZF protein domain comprises an array of one to ten zinc finger motifs.
5. The engineered cell of any one of embodiments 1 to 4, wherein the ZF protein domain comprises an array of six zinc finger motifs.
6. The engineered cell of any one of embodiments 1 to 5, wherein each ligand binding domain comprises a single-domain antibody.
7. The engineered cell of embodiment 6, wherein the single-domain antibody comprises a V_{H}H.
8. The engineered cell of embodiment 7, wherein the V_{H}H is an anti-caffeine V_{H}H (ac V_{H}H).
9. The engineered cell of embodiment 8, wherein the anti-caffeine V_{H}H (ac V_{H}H) comprises the amino acid sequence of SEQ ID NO: 52.
10. The engineered cell of embodiment 8 or embodiment 9, wherein each of the first ligand binding domain and the second ligand binding domain comprises the ac V_{H}H.
11. The engineered cell of embodiment 7, wherein the cognate ligand is a cannabidiol or a phytocannabinoid, and the V_{H}H comprises an amino acid sequence selected from the group consisting of SEQ ID NO: 53, SEQ ID NO: 54, SEQ ID NO: 55, SEQ ID NO: 56, and SEQ ID NO: 57 (CA-14, DB-6, DB-11, DB-18, and DB-21, respectively).
12. The engineered cell of embodiment 12, wherein one of the first or the second ligand binding domain comprises a V_{H}H comprising the amino acid sequence of SEQ ID NO: 53 (CA-14) and the other of the first or the second binding domain comprises a V_{H}H comprising an amino acid sequence selected from the group consisting of: SEQ ID NO: 54, SEQ ID NO: 55, SEQ ID NO: 56, and SEQ ID NO: 57 (DB-6, DB-11, DB-18, and DB-21, respectively).
13. The engineered cell of embodiment 12, wherein one of the first or the second ligand binding domain comprises a V_{H}H comprising the amino acid sequence of SEQ ID NO: 53 (CA-14) and the other of the first or the second binding domain comprises a V_{H}H comprising the amino acid sequence of SEQ ID NO: 57 (DB-21).
14. The engineered cell of any one of embodiments 1 to 5, wherein each ligand binding domain comprises an anti-nicotine antibody heavy chain variable domain (anti-Nic VH) or an anti-nicotine antibody light chain variable domain (anti-Nic VL).
15. The engineered cell of embodiment 14, wherein the anti-Nic VH comprises the amino acid sequence of SEQ ID NO: 58.
16. The engineered cell of embodiment 14 or embodiment 15, wherein the anti-Nic VL comprises the amino acid sequence of SEQ ID NO: 59.
17. The engineered cell of any one of embodiments 14 to 16, wherein one of the first or the second ligand binding domain comprises the anti-Nic VH and the other of the first or the second ligand binding domain comprises the anti-Nic VL.
18. An engineered cell comprising an inducible transcription factor (ITF), wherein the ITF comprises:
   a first monomer, wherein the first monomer comprises a DNA binding domain and a first ligand binding domain; and
   a second monomer, wherein the second monomer comprises a transcriptional effector domain and a second ligand binding domain,
   wherein each monomer is oligomerizable via binding of a cognate ligand that binds to each ligand binding domain,
   wherein ligand-induced oligomerization forms the ITF,
   wherein the DNA binding domain comprises a zinc finger (ZF) protein domain that is modular and comprises an array of six ZF motifs, and
   wherein the cognate ligand is selected from the group consisting of: rapamycin, AP1903, AP20187, FK1012, derivatives thereof, and analogs thereof.
19. The engineered cell of embodiment 18, wherein one of the first or the second ligand binding domain comprises an FKBP domain, and the other of the first or the second ligand binding domain comprises an FRB domain.
20. The engineered cell of embodiment 19, wherein the FKBP comprises the amino acid sequence of SEQ ID NO: 60.
21. The engineered cell of embodiment 19 or embodiment 20, wherein the FRB domain comprises the amino acid sequence of SEQ ID NO: 61.
22. The engineered cell of any one of embodiments 19 to 21, wherein the first ligand binding domain comprises the FKBP domain and the second ligand binding domain comprises the FRB domain.
23. The engineered cell of any one of embodiments 19 to 22, wherein one of the first or the second ligand binding domain comprises the FRB domain, and the other of the first or the second ligand binding domain comprises the FKBP domain.
24 The engineered cell of any one of embodiments 1 to 23, wherein the first monomer and/or the second monomer further comprises a nuclear localization signal (NLS).
25. The engineered cell of embodiment 24, wherein the NLS comprises an amino acid sequence selected from the group consisting of: SEQ ID NO: 2, SEQ ID NO: 3, and SEQ ID NO: 4.
26. The engineered cell of any one of embodiments 1 to 25, wherein the second monomer further comprises a nuclear export signal (NES).
27. The engineered cell of embodiment 26, wherein the NES comprises an amino acid sequence selected from the group consisting of: SEQ ID NO: 5, SEQ ID NO: 6, SEQ ID NO: 7 SEQ ID NO: 8, SEQ ID NO: 9, SEQ ID NO: 10, SEQ ID NO: 11, SEQ ID NO: 12, SEQ ID NO: 13, and SEQ ID NO: 14.
28. The engineered cell of any one of embodiments 1 to 27, wherein the ITF is capable of modulating transcription of a gene of interest operably linked to an ITF-responsive promoter.
29. An expression system comprising:
   a first expression cassette comprising a first promoter and an exogenous polynucleotide sequence encoding a first monomer, wherein the first monomer comprises a DNA binding domain and a first ligand binding domain; and
   a second expression cassette comprising a second promoter and an exogenous polynucleotide sequence encoding a second monomer, wherein the second monomer comprises a transcription effector domain and a second ligand binding domain,
   wherein each monomer is oligomerizable via a cognate ligand that binds to each ligand binding domain,
   wherein ligand-induced oligomerization of the first and the second monomer forms an inducible transcription factor (ITF), and
   wherein the cognate ligand is selected from the group consisting of: caffeine, a cannabidiol (CBD), a phytocannabinoid, and nicotine.
30. The expression system of embodiment 29, wherein the DNA binding domain comprises a zinc finger (ZF) protein domain.
31. The expression system of embodiment 30, wherein the ZF protein domain is modular in design and is composed of one or more zinc finger arrays (ZFA).
32. The expression system of embodiment 31, wherein the ZF protein domain comprises an array of one to ten zinc finger motifs.
33. The expression system of embodiments 29 to 32, wherein the ZF protein domain comprises an array of six zinc finger motifs.
34. The expression system of any one of embodiments 29 to 33, wherein each ligand binding domain comprises a single-domain antibody.
35. The expression system of embodiment 34, wherein the single-domain antibody comprises a V_{H}H.
36. The expression system of embodiment 35, wherein the V_{H}H is an anti-caffeine V_{H}H (ac V_{H}H).
37. The expression system of embodiment 36, wherein the anti-caffeine V_{H}H (ac V_{H}H) comprises the amino acid sequence of SEQ ID NO: 52.
38. The expression system of embodiment 36 or embodiment 37, wherein each of the first ligand binding domain and the second ligand binding domain comprises the ac V_{H}H.
39. The expression system of embodiment 38, wherein the first monomer and second monomer are capable of forming a homodimer upon binding to caffeine.
40. The expression system of embodiment 35, wherein the cognate ligand is a cannabidiol or a phytocannabinoid, and the V_{H}H comprises an amino acid sequence selected from the group consisting of SEQ ID NO: 53, SEQ ID NO: 54, SEQ ID NO: 55, SEQ ID NO: 56, and 57 (CA14, DB6, DB11, DB18, and DB21, respectively).
41. The expression system of embodiment 40, wherein the first ligand binding domain comprises a V_{H}H comprising the amino acid sequence of SEQ ID NO: 53 (CA-14) and the second binding domain comprises a V_{H}H comprising an amino acid sequence selected from the group consisting of: 54, SEQ ID NO: 55, SEQ ID NO: 56, and 57 (DB6, DB11, DB18, and DB21, respectively).
42. The expression system of embodiment 40, wherein the first ligand binding domain comprises a V_{H}H comprising the amino acid sequence of SEQ ID NO: 53 (CA-14) and the second binding domain comprises a V_{H}H comprising the amino acid sequence of SEQ ID NO: 57 (DB-21).
43. The expression system of any one of embodiments 40 to 42, wherein the first monomer and second monomer are capable of forming a heterodimer upon binding to a cannabidiol or a phytocannabinoid.
44. The expression system of any one of embodiments 29 to 33, wherein each ligand binding domain comprises an anti-nicotine antibody heavy chain variable domain (anti-Nic VH) or an anti-nicotine antibody light chain variable domain (anti-Nic VL).
45. The expression system of embodiment 44, wherein the anti-Nic VH comprises the amino acid sequence of SEQ ID NO: 58.
46. The expression system of embodiment 44 or 45, wherein the anti-Nic VL comprises the amino acid sequence of SEQ ID NO: 59.
47. The expression system of any one of embodiments 44 to 46, wherein the first ligand binding domain comprises the anti-Nic VH and the second binding domain comprises the anti-Nic VL.
48. The expression system of any one of embodiments 44 to 46, wherein the first ligand binding domain comprises the anti-Nic VL and the second ligand binding domain comprises the anti-Nic VH.
49. The expression system of any one of embodiments 44 to 48, wherein the first monomer and second monomer are capable of forming a heterodimer upon binding to nicotine.
50. The expression system of any one of embodiments 29 to 49, further comprising a third expression cassette comprising an ITF-responsive promoter operably linked to a gene of interest, wherein the ITF-responsive promoter comprises a core promoter sequence and a sequence that binds to the DNA binding domain of the first engineered polypeptide.
51. The expression system of embodiment 50, wherein the core promoter sequence comprises a minimal promoter.
52. The expression system of embodiment 50 or embodiment 51, wherein the DNA binding domain comprises a zinc finger (ZF) protein domain, and the sequence that binds to the DNA binding domain comprises one or more ZF binding sites.
53. The expression system of embodiment 52, wherein the ZF protein domain comprises an array of six zinc finger motifs, and the sequence that binds to the DNA binding domain comprises at least one binding site for the six zinc finger motifs.
54. An expression system comprising:
   a first expression cassette comprising a first promoter and an exogenous polynucleotide sequence encoding a first monomer, wherein the first monomer comprises a DNA binding domain and a first ligand binding domain; and
   a second expression cassette comprising a second promoter and an exogenous polynucleotide sequence encoding a second monomer, wherein the second monomer comprises a transcriptional effector domain and a second ligand binding domain,
   wherein each monomer is oligomerizable via binding of a cognate ligand that binds to each ligand binding domain,
   wherein ligand-induced oligomerization of the first and the second monomer forms an inducible transcription factor (ITF),
wherein the DNA binding domain comprises a zinc finger protein domain that is modular and comprises ten zinc finger arrays (ZFA), and
   wherein the cognate ligand is selected from the group consisting of: rapamycin, AP1903, AP20187, FK1012, derivatives thereof, and analogs thereof.
55. The expression system of embodiment 54, wherein each ligand binding domain comprises an FKBP domain or an FRB domain.
56. The expression system of embodiment 55, wherein the FKBP comprises the amino acid sequence of SEQ ID NO: 60.
57. The expression system of embodiment 55 or embodiment 56, wherein the FRB domain comprises the amino acid sequence of SEQ ID NO: 61.
58. The expression system of any one of embodiments 55 to 57, wherein the first ligand binding domain comprises the FKBP domain and the second ligand binding domain comprises the FRB domain.
59. The expression system of any one of embodiments 55 to 57, wherein the first ligand binding domain comprises the FRB domain and the second ligand binding domain comprises the FKBP domain.
60. The expression system of any one of embodiments 55 to 59, wherein the first monomer and the second monomer are capable of forming a heterodimer upon binding to a ligand selected from the group consisting of: rapamycin, AP1903, AP20187, FK1012, derivatives thereof, or analogs thereof.
61. The expression system of any one of embodiments 29 to 60, wherein the first monomer and/or the second monomer further comprises a nuclear localization signal (NLS).
62. The expression system of embodiment 61, wherein the NLS comprises an amino acid sequence selected from the group consisting of: SEQ ID NO: 2, SEQ ID NO: 3, and SEQ ID NO: 4.
63. The expression system of any one of embodiments 29 to 62, wherein the second monomer further comprises a nuclear export signal (NES).
64. The expression system of embodiment 63, wherein the NES comprises an amino acid sequence selected from the group consisting of: SEQ ID NO: 5, SEQ ID NO: 6, SEQ ID NO: 7 SEQ ID NO: 8, SEQ ID NO: 9, SEQ ID NO: 10, SEQ ID NO: 11, SEQ ID NO: 12, SEQ ID NO: 13, and SEQ ID NO: 14.
65. The expression system of any one of embodiments 29 to 64, wherein the ITF is capable of modulating transcription of a gene of interest operably linked to an ITF-responsive promoter.
66. The expression system of any one of embodiments 29 to 65, wherein the first and/or the second promoter is a constitutive promoter or a regulatable promoter.
67. The expression system of any one of embodiments 29 to 66, wherein the first and/or the second promoter is a synthetic promoter.
68. The expression system of embodiments 29 to 65, wherein the first promoter and/or the second promoter is a constitutive promoter selected from the group consisting of: CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, and hUBIb.
69. The expression system of any one of embodiments 29 to 68, further comprising a third expression cassette comprising an ITF-responsive promoter operably linked to a gene of interest, wherein the third promoter comprises a core promoter sequence and a sequence that binds to the DNA binding domain of the first engineered polypeptide.
70. The expression system of embodiment 69, wherein the core promoter sequence comprises a minimal promoter.
71. The expression system of embodiment 70, wherein the minimal promoter is selected from the group consisting of: minP, minCMV, YB_TATA, minTATA, and minTK.
72. The expression system of embodiment 69, wherein the core promoter sequence is derived from a constitutive promoter.
73. The expression system of embodiment 72, wherein the core promoter sequence is derived from a constitutive promoter selected from the group consisting of: CMV, EFS, SFFV, SV40, MND, PGK, UbC, EF1a, hCAGG, hACTb, heIF4A1, hGAPDH, hGRP78, hGRP94, hHSP70, hKINb, and hUBIb.
74. The expression system of any one of embodiments 29 to 73, wherein the transcriptional effector domain comprises a transcriptional activation domain.
75. The expression system of embodiment 74, wherein the transcriptional activation domain is selected from the group consisting of: a Herpes Simplex Virus Protein 16 (VP16) activation domain; an activation domain comprising four tandem copies of VP16; a VP64 activation domain; a p65 activation domain of NFκB; an Epstein-Barr virus R transactivator (Rta) activation domain; a tripartite activator comprising the VP64, the p65, and the Rta activation domains (VPR activation domain); and a histone acetyltransferase (HAT) core domain of the human E1A-associated protein p300 (p300 HAT core activation domain).
76. The expression system of any one of embodiments 29 to 73, wherein the transcriptional effector domain comprises a transcriptional repressor domain.
77. The expression system of embodiment 76, wherein the transcriptional repressor domain is selected from the group consisting of: a Krüppel associated box (KRAB) repression domain; a Histone Deacetylase 4 (HDAC4) repressor domain; a Scleraxis (SCX) HLH domain, an Inhibitor of DNA binding 1 (ID1) HLH domain, a HECT domain and RCC1-like domain-containing protein 2 (HERC2) Cyt-b5 domain, a Twist-related protein 1 (TWST1) HLH domain, an Homeobox protein Nkx-2.2 (NKX22) homeodomain, an Inhibitor of DNA binding 1 (ID3) HLH domain, and a Twist-related protein 2 (TWST2) HLH domain, an EED repressor domain; a Repressor Element Silencing Transcription Factor (REST) repression domain; a WRPW motif (SEQ ID NO: 76) of the hairy-related basic helix-loop-helix repressor proteins, the motif is known as a WRPW (SEQ ID NO: 76) repression domain; a DNA (cytosine-5)-methyltransferase 3B (DNMT3B) repression domain; and an HP1 alpha chromoshadow repression domain.
78. The expression system of any one of embodiments 29 to 77, wherein the DNA binding domain binds to the ITF-responsive promoter.
79. An engineered cell comprising the expression system of any one of embodiments 29 to 53.
80. An engineered cell comprising the expression system of any one of embodiments 54 to 70.
81. The engineered cell of any one of embodiments 1 to 28, 79 or 80, comprising a human cell.
82. The engineered cell of any one of embodiments 1 to 28 or 79 to 81, comprising a stem cell.
83. The engineered cell of any one of embodiments 1 to 28 or 79 to 81, comprising an immune cell.
84 The engineered cell of any one of embodiments 1 to 28 or 79 to 81, wherein the cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an erythrocyte, a platelet cell, a human embryonic stem cell (ESC), an ESC-derived cell, a pluripotent stem cell, a mesenchymal stromal cell (MSC), an induced pluripotent stem cell (iPSC), and an iPSC-derived cell.
85. A genetic switch for modulating transcription of a gene of interest, comprising:
   the engineered cell of any one of embodiments 8 to 10; and
   an effective amount of caffeine.
86. A genetic switch for modulating transcription of a gene of interest, comprising:
   the engineered cell of any one of embodiments 11 to 13; and
   an effective amount of cannabidiol (CBD) or a phytocannabinoid.
87. A genetic switch for modulating transcription of a gene of interest, comprising:
   the engineered cell of any one of embodiments 14 to 17; and
   an effective amount of nicotine.
88. A genetic switch for modulating transcription of a gene of interest, comprising:
   the engineered cell of any one of embodiments 18 to 23, and
   and effective amount of rapamycin.
89. A method of modulating transcription of a gene of interest, comprising:
   contacting the engineered cell of embodiment 81 with a cognate ligand selected from the group consisting of: caffeine, a cannabidiol (CBD), a phytocannabinoid, and nicotine, to induce formation of the inducible transcription factor (ITF).
90. The method of embodiment 89, further comprising culturing the engineered cell for a time period sufficient to allow the ITF to modulate transcription of a gene operably linked to an ITF-responsive promoter.
91. The method of embodiment 89, wherein the contacting is performed in a human or animal.
92. The method of embodiment 91, wherein contacting the transformed cell with the cognate ligand comprises administering a pharmacological dose of the cognate ligand to the human or animal.
93. The method of any one of embodiments 89 to 92, wherein the cognate ligand comprises caffeine.
94. The method of any one of embodiments 89 to 92, wherein the cognate ligand is cannabidiol (CBD)
95 The method of any one of embodiments 89 to 92, wherein the cognate ligand is nicotine.
96. A method of modulating transcription of a gene of interest, comprising: contacting the engineered cell of embodiment 80 with rapamycin to induce formation of the inducible transcription factor (ITF).
97. The method of embodiment 96, wherein the method further comprises culturing the engineered cell for a time period sufficient to allow the ITF to modulate transcription of a gene operably linked to an ITF-responsive promoter.
98. The method of embodiment 96, wherein the engineered cell is in a human or animal, and wherein contacting the engineered cell with the cognate ligand comprises administering a pharmacological dose of the rapamycin to the human or animal.
99. The method of any one of embodiments 96 to 98, wherein the transcription effector domain of the second monomer comprises a transcriptional activation domain, and wherein modulating transcription comprises activating transcription of the gene of interest.
100. The method of any one of embodiments 96 to 99, wherein the transcription effector domain of the second monomer comprises a transcriptional repressor domain, wherein modulating transcription comprises repressing transcription of the gene of interest.
101. An engineered cell comprising an inducible transcription factor (ITF),
   wherein the ITF comprises a DNA binding domain, a transcriptional effector domain, and a ligand binding domain,
   wherein the ITF undergoes nuclear localization upon binding of the ligand binding domain to a cognate ligand,
   wherein when localized to a cell nucleus, the ITF is capable of modulating transcription of a gene of interest operably linked to an ITF-responsive promoter, and
   wherein the cognate ligand is mifepristone or a derivative thereof.
102. The engineered cell of embodiment 101, wherein the ligand binding domain comprises a progesterone receptor domain.
103. The engineered cell of embodiment 102, wherein the progesterone receptor domain comprises the amino acid sequence of SEQ ID NO: 68.
104. The engineered cell of any one of embodiments 101 to 103, wherein the DNA binding domain comprises a zinc finger (ZF) protein domain.
105. The engineered cell of embodiment 104, wherein the ZF protein domain is modular in design and is composed of one or more zinc finger arrays (ZFA).
106. The engineered cell of embodiment 105, wherein the ZF protein domain comprises one to ten zinc finger motifs.
107. The engineered cell of any one of embodiments 104 to 106, wherein the ZF protein domain comprises six zinc finger motifs.
108. The engineered cell of any one of embodiments 101 to 107, wherein the ITF further comprises a peptide linker between the DNA binding domain and the transcriptional effector domain.
109. The engineered cell of any one of embodiments 101 to 108, wherein the DNA binding domain binds to the ITF-responsive promoter.
110. The engineered cell of any one of embodiments 101 to 109, wherein the ITF-responsive promoter comprises an ITF-binding domain sequence and a core promoter sequence.
111. The engineered cell of embodiment 110, wherein the core promoter sequence comprises a minimal promoter.
112. The engineered cell of embodiment 111, wherein the minimal promoter is selected from the group consisting of: minP, minCMV, YB_TATA, minTATA, and minTK.
113. The engineered cell of embodiment 110, wherein the core promoter sequence is derived from a constitutive promoter.
114. The engineered cell of embodiment 113, wherein the core promoter sequence is derived from a constitutive promoter selected from the group consisting of: CMV, EFS, SFFV, SV40, MND, PGK, UbC, EF1a, hCAGG, hACTb, heIF4A1, hGAPDH, hGRP78, hGRP94, hHSP70, hKINb, and hUBIb.
115. The engineered cell of any one of embodiments 101 to 114, wherein the cell further comprises an expression cassette comprising the ITF-responsive promoter operably linked to an exogenous polynucleotide sequence encoding the gene of interest.
116. The engineered cell of any one of embodiments 101 to 115, wherein the transcriptional effector domain comprises a transcriptional activation domain.
117. The engineered cell of embodiment 116, wherein the transcriptional activation domain is selected from the group consisting of: a Herpes Simplex Virus Protein 16 (VP16) activation domain; an activation domain comprising four tandem copies of VP16; a VP64 activation domain; a p65 activation domain of NFκB; an Epstein-Barr virus R transactivator (Rta) activation domain; a tripartite activator comprising the VP64, the p65, and the Rta activation domains (VPR activation domain); and a histone acetyltransferase (HAT) core domain of the human E1A-associated protein p300 (p300 HAT core activation domain).
118. The engineered cell of any one of embodiments 101 to 115, wherein the transcriptional effector domain comprises a transcriptional repressor domain.
119. The engineered cell of embodiment 118, wherein the transcriptional repressor domain is selected from the group consisting of: a Krüppel associated box (KRAB) repression domain; a truncated Krüppel associated box (KRAB) repression domain; a Histone Deacetylase 4 (HDAC4) repressor domain; a Scleraxis (SCX) HLH domain, an Inhibitor of DNA binding 1 (ID1) HLH domain, a HECT domain and RCC1-like domain-containing protein 2 (HERC2) Cyt-b5 domain, a Twist-related protein 1 (TWST1) HLH domain, an Homeobox protein Nkx-2.2 (NKX22) homeodomain, an Inhibitor of DNA binding 1 (ID3) HLH domain, and a Twist-related protein 2 (TWST2) HLH domain, and EED repressor domain; a Repressor Element Silencing Transcription Factor (REST) repression domain; a WRPW motif (SEQ ID NO: 76) of the hairy-related basic helix-loop-helix repressor proteins, the motif is known as a WRPW (SEQ ID NO: 76) repression domain; a DNA (cytosine-5)-methyltransferase 3B (DNMT3B) repression domain; and an HP1 alpha chromoshadow repression domain.
120. An expression system comprising a first expression cassette comprising a first promoter and an exogenous polynucleotide sequence encoding an inducible transcription factor (ITF), wherein the ITF comprises a DNA binding domain, a transcriptional effector domain, and a ligand binding domain,
   wherein the ITF undergoes nuclear localization upon binding of the ligand binding domain to a cognate ligand,
   wherein when localized to a cell nucleus, the ITF is capable of modulating transcription of a gene of interest operably linked to an ITF-responsive promoter, and
   wherein the cognate ligand is mifepristone or a derivative thereof.
121. The expression system of embodiment 120, wherein the ligand binding domain comprises a progesterone receptor domain.
122. The expression system of embodiment 121, wherein the progesterone receptor domain comprises the amino acid sequence of SEQ ID NO: 68.
123. The expression system of any one of embodiments 120 to 122, wherein the DNA binding domain comprises a zinc finger (ZF) protein domain.
124. The expression system of embodiment 123, wherein the ZF protein domain is modular in design and is composed of one or more zinc finger arrays (ZFA).
125. The expression system of embodiment 124, wherein the ZF protein domain comprises one to ten zinc finger motifs.
126. The expression system of embodiments 120 to 125, wherein the ZF protein domain comprises six zinc finger motifs.
127. The expression system of any one of embodiments 120 to 126, wherein the ITF further comprises a linker localized between the DNA binding domain and the transcriptional effector domain.
128. The expression system of any one of embodiments 120 to 127, wherein the transcriptional effector domain comprises a transcriptional activation domain.
129. The expression system of embodiment 128, wherein the transcriptional activation domain is selected from the group consisting of: a Herpes Simplex Virus Protein 16 (VP16) activation domain; an activation domain comprising four tandem copies of VP16; a VP64 activation domain; a p65 activation domain of NFκB; an Epstein-Barr virus R transactivator (Rta) activation domain; a tripartite activator comprising the VP64, the p65, and the Rta activation domains (VPR activation domain); and a histone acetyltransferase (HAT) core domain of the human E1A-associated protein p300 (p300 HAT core activation domain).
130. The expression system of any one of embodiments 120 to 127, wherein the transcriptional effector domain comprises a transcriptional repressor domain.
131. The expression system of embodiment 130, wherein the transcriptional repressor domain is selected from the group consisting of: a Krüppel associated box (KRAB) repression domain; a Histone Deacetylase 4 (HDAC4) repressor domain; a Scleraxis (SCX) HLH domain, an Inhibitor of DNA binding 1 (ID1) HLH domain, a HECT domain and RCC1-like domain-containing protein 2 (HERC2) Cyt-b5 domain, a Twist-related protein 1 (TWST1) HLH domain, an Homeobox protein Nkx-2.2 (NKX22) homeodomain, an Inhibitor of DNA binding 1 (ID3) HLH domain, and a Twist-related protein 2 (TWST2) HLH domain, an EED repressor domain; a Repressor Element Silencing Transcription Factor (REST) repression domain; a WRPW motif (SEQ ID NO: 76) of the hairy-related basic helix-loop-helix repressor proteins, the motif is known as a WRPW (SEQ ID NO: 76) repression domain; a DNA (cytosine-5)-methyltransferase 3B (DNMT3B) repression domain; and an HP1 alpha chromoshadow repression domain.
132. The expression system of any one of embodiments 120 to 131, wherein the DNA binding domain binds to the ITF-responsive promoter.
133. The expression system of any one of embodiments 120 to 132, wherein the first promoter is a constitutive promoter, a regulatable promoter, or a synthetic promoter.
134. The expression system of embodiment 133, wherein the first promoter is a constitutive promoter selected from the group consisting of: CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, and hUBIb.
135. The expression system of any one of embodiments 120 to 134, further comprising an expression cassette comprising the ITF-responsive promoter operably linked to an exogenous polynucleotide sequence encoding the gene of interest.
136. The expression system of embodiment 135, wherein the ITF-responsive promoter comprises an ITF-binding domain sequence and a core promoter sequence.
137. The expression system of embodiment 136, wherein the core promoter sequence comprises a minimal promoter.
138. The expression system of embodiment 137, wherein the minimal promoter is selected from the group consisting of: minP, minCMV, YB_TATA, minTATA, and minTK.
139. The expression system of embodiment 138, wherein the core promoter sequence is derived from a constitutive promoter.
140. The expression system of embodiment 139, wherein the core promoter sequence is derived from a constitutive promoter selected from the group consisting of: CMV, EFS, SFFV, SV40, MND, PGK, UbC, EF1a, hCAGG, hACTb, heIF4A1, hGAPDH, hGRP78, hGRP94, hHSP70, hKINb, and hUBIb.
141. An engineered cell comprising the expression system of any one of embodiments 120 to 140.
142. The engineered cell of any one of embodiments 101 to 119 or 141, comprising a human cell.
143. The engineered cell of any one of embodiments 101 to 119, 141, or 142, comprising a stem cell.
144. The engineered cell of any one of embodiments 1 to 119, 141, or 142, comprising an immune cell.
145. The engineered cell of any one of embodiments 101 to 119, 141, or 142, wherein the cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an erythrocyte, a platelet cell, a human embryonic stem cell (ESC), an ESC-derived cell, a pluripotent stem cell, a mesenchymal stromal cell (MSC), an induced pluripotent stem cell (iPSC), and an iPSC-derived cell.
146. A genetic switch for modulating transcription of a gene of interest, comprising:
   the engineered cell of any one of embodiments 101 to 119 or 141 to 145; and
   an effective amount of mifepristone.
147. A method of modulating transcription of a polypeptide of interest, comprising:
   contacting the engineered cell of any one of embodiments 101 to 119 or 142 to 146 with mifepristone to induce nuclear localization of the inducible transcription factor (ITF).
148. The method of embodiment 147, wherein the method further comprises culturing the engineered cell for a time period sufficient to allow the ITF to modulate transcription of a gene operably linked to an ITF-responsive promoter.
149. The method of embodiment 147, wherein the engineered cell is in a human or animal, and wherein contacting the engineered cell with the mifepristone comprises administering a pharmacological dose of the mifepristone to the human or animal.
150. The method of any one of embodiments 147 to 149, wherein the transcriptional effector domain comprises a transcriptional activation domain, and wherein modulating transcription comprises activating transcription of the gene of interest.
151. The method of any one of embodiments 147 to 149, wherein the transcriptional effector domain comprises a transcriptional repressor domain, and wherein modulating transcription comprises repressing transcription of the gene of interest.

### EXAMPLES

Below are examples of specific embodiments for carrying out the present disclosure. The examples are offered for illustrative purposes only, and are not intended to limit the scope of the present disclosure in any way. Efforts have been made to ensure accuracy with respect to numbers used (e.g., amounts, temperatures, etc.), but some experimental error and deviation should, of course, be allowed for.

The practice of the present disclosure will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B (1992).

### Example 1: Transcription Modulation by Drug-Induced Transcription Factors

Transduction assays were performed to assess mCherry expression in response to inducible transcription factors activated by caffeine, rapamycin, CBD, or nicotine.

### Materials and Methods

U87-MG cells were transduced with a lentivirus encoding a synthetic ITF-responsive promoter (SEQ ID NO: 65) composed of 4×ZF binding sites that bind to ZF10 linked to YBTATA, a minimal promoter. The promoter drives expression of mCherry. This cell line was called “reporter cells.” Caffeine-inducible transcription factor constructs included a first monomer construct (construct 1) composed of an anti-caffeine V_{H}H (“aCaff”), a zinc finger protein domain of SEQ ID NO: 1 (ZF10), a nuclear localization signal of SEQ ID NO: 2, a cleavable linker, and a blue fluorescent protein tag, and a second monomer construct (construct 2) composed of a nuclear export signal (NES) of SEQ ID NO: 5, a VPR transcriptional activation domain, an anti-caffeine V_{H}H (“aCaff”), a cleavable linker, and a green fluorescent protein tag. Rapamycin-inducible transcription factor constructs included a first monomer construct (construct 3) composed of an FKBP domain, a zinc finger protein domain of SEQ ID NO: 1, a nuclear localization signal of SEQ ID NO: 2, a cleavable linker, and a blue fluorescent protein tag (construct 1), and a second monomer construct (construct 4) composed of a nuclear export signal (NES) of SEQ ID NO: 5, a VPR transcriptional activation domain, an FRB domain, a cleavable linker, and a green fluorescent protein tag. Cannabinoid-inducible transcription factor constructs included a first monomer construct (construct 5) composed of a first anti-CBD V_{H}H (CA-14), a zinc finger protein domain of SEQ ID NO: 1, a nuclear localization signal of SEQ ID NO: 2, a cleavable linker, and a blue fluorescent protein tag, and a second monomer construct (construct 6) composed of a nuclear export signal (NES) of SEQ ID NO: 5, a VPR transcriptional activation domain, a second anti-CBD V_{H}H that is oligomerizable with the first anti-CBD V_{H}H (DB-21), a cleavable linker, and a green fluorescent protein tag. Nicotine-inducible transcription factor constructs included a first monomer construct (construct 7) composed of an anti-nicotine VH, a zinc finger protein domain of SEQ ID NO: 1, a nuclear localization signal of SEQ ID NO: 2, a cleavable linker, and a blue fluorescent protein tag, and a second monomer construct (construct 8) composed of a nuclear export signal (NES) of SEQ ID NO: 5, a VPR transcriptional activation domain, an anti-nicotine VL that is oligomerizable with the anti-nicotine VH of the first construct, a cleavable linker, and a green fluorescent protein tag.

On day 1, reporter cells were transduced with lentivirus(es) encoding the DNA constructs shown in Table 4.

TABLE 4
Constructs used for transducing reporter cells

| Cell | Construct | |
|---|---|---|
| Groups | No. | Architecture |
| A | 1 | aCaff V_{H}H - ZF10_NLS - E2A-T2A - BFP |
| | 2 | NES_VPR - aCaff V_{H}H - E2A-T2A - GFP |
| B | 3 | FKBP - ZF10_NLS - E2A-T2A - BFP |
| | 4 | NES_VPR - FRB - E2A-T2A - GFP |
| C | 5 | CA-14 CBD V_{H}H - ZF10_NLS - E2A-T2A - BFP |
| | 6 | NES_VPR - DB-21 CBD V_{H}H - E2A-T2A - GFP |
| D | 7 | Nic VH - ZF10_NLS - E2A-T2A - BFP |
| | 8 | NES_VPR - Nic VL - E2A-T2A - GFP |

To assay for transcription modulation regulated by caffeine, rapamycin, a cannabidiol (CBD), and nicotine, on day 2, cell groups A-D and a control cell group (untransduced) were split into a 48-well plate with caffeine, rapamycin, CBD, or nicotine drug treatments as shown in Table 5. Cell population E was frozen for drug treatment on a later date.

TABLE 5
Assay Conditions

| | Conc. + | Conc. + | Conc. + | Conc. + | Conc. + | Conc. + |
|---|---|---|---|---|---|---|
| Cell Groups | Drug | Drug | Drug | Drug | Drug | Drug |
| A | 100 uM | 10 uM | 1 uM | 0.1 uM | 0.01 uM | No drug |
| | caffeine | caffeine | caffeine | caffeine | caffeine | |
| B | 1000 nM | 100 nM | 10 nM | 1 nM | No drug | — |
| | rapamycin | rapamycin | rapamycin | rapamycin | | |
| C | 1000 nM | 100 nM | 10 nM | No drug | — | — |
| | CBD | CBD | CBD | | | |
| D | 10 uM | 1 uM | 0.1 uM | No drug | — | — |
| | nicotine | nicotine | nicotine | | | |
| Control | 100 uM | 1000 nM | 1000 nM | 10 uM | No drug | No drug |
| (untransduced) | caffeine | rapamycin | CBD | nicotine | | |

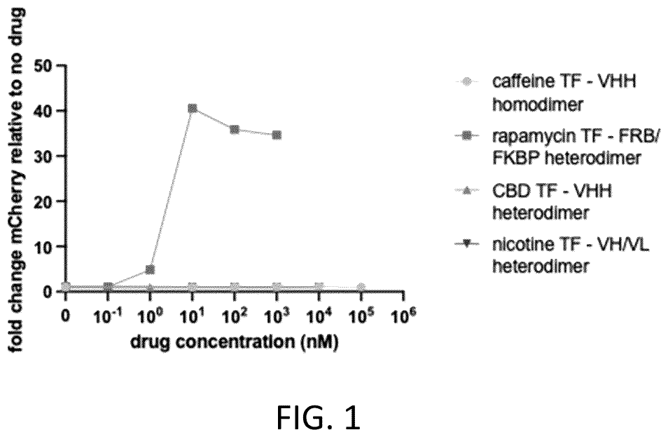

On day 4, media was removed from the 48-well plate and cells were trypsinized and then resuspended in fluorescence-activated cell sorting (FACS) buffer plus Sytox Red (fluoresces in APC channel) viability dye. Cells were run on flow cytometer and gated by FSC/SSC for cells, FSC/Sytox Red—for live cells, green fluorescent protein (GFP)/blue fluorescent protein (BFP)+ for transduced cells. mCherry geometric mean fluorescent intensity (gMFI) was measured at each concentration and normalized to no drug for cell groups A-D and the control cell group, as shown in FIG. 1.

### Results

As shown in FIG. 1, the rapamycin-regulated transcription modulation system both induced mCherry expression in the presence of drug in a concentration-dependent manner.

### Example 2: Transcription Modulation Systems Including Two Nuclear Localization Signals

Transduction assays were performed to assess transcriptional modulation activity of caffeine-, CBD-, and nicotine-inducible transcription factors with the first monomer and the second monomer each including a nuclear localization signal (NLS).

### Materials and Methods

U87MG cells transduced with a 4×ZF10-1 BS_minYBTATA:mCherry vector (referred to as “reporter cells”) were plated at 6e5/cells in a 6 well plate on day 0.

Caffeine-inducible transcription factor constructs were designed and included a first monomer construct (construct 10) composed of an anti-caffeine V_{H}H (“aCaff”), a zinc finger protein domain of SEQ ID NO: 1 (“ZF10”), a 3×FLAG-tagged nuclear localization signal (NLS) of SEQ ID NO: 2, a cleavable linker, and a blue fluorescent protein tag (BFP), and a second monomer construct (construct 11) composed of a 3×FLAG-tagged NLS of SEQ ID NO: 2, a VPR transcriptional activation domain, an anti-caffeine V_{H}H (“aCaff”), a cleavable linker, and a green fluorescent protein tag (GFP). Nicotine-inducible transcription factor constructs were designed and included a first monomer construct (construct 12) composed of an anti-nicotine VH, a zinc finger protein domain of SEQ ID NO: 1, an NLS of SEQ ID NO: 2, a cleavable linker, and a BFP tag, and a second monomer construct (construct 13) composed of a 3×FLAG-tagged nuclear localization signal (NLS) of SEQ ID NO: 2, a VPR transcriptional activation domain, an anti-nicotine VL that is oligomerizable with the anti-nicotine VH of the first construct, a cleavable linker, and a GFP tag. Cannabinoid-inducible transcription factor constructs included a first monomer construct (construct 14) composed of a first anti-CBD V_{H}H (CA-14), a zinc finger protein domain of SEQ ID NO: 1, a nuclear localization signal of SEQ ID NO: 2, a cleavable linker, and a BFP tag, and a second monomer construct (construct 15) composed of a 3×FLAG-tagged nuclear localization signal (NLS) of SEQ ID NO: 2, a VPR transcriptional activation domain, a second anti-CBD V_{H}H that is oligomerizable with the first anti-CBD V_{H}H (DB-21), a cleavable linker, and a GFP tag.

On day 1, the reporter cells were co-transduced with lentiviruses encoding the DNA constructs shown in Table 6.

TABLE 6
Constructs used for transducing reporter cells

| Cell | Construct | |
|---|---|---|
| Groups | No. | Architecture |
| F | 10 | aCaff V_{H}H_ZF10_3 × Flag NLS_dual 2A_BFP |
| | 11 | 3 × Flag NLS_VPR_aCaff V_{H}H_dual 2A_GFP |
| G | 12 | aNic VH_ZF10_3 × Flag NLS_dual 2A_BFP |
| | 13 | 3 × Flag NLS_VPR_aNic VL_dual 2A_GFP |
| H | 14 | CA-14_ZF10_3 × Flag NLS_dual 2A_BFP |
| | 15 | 3 × Flag NLS_VPR_DB-21 dual 2A_GFP |

On day 2, cells of each cell group F-H were split and treated with 10 uM, 1 uM, 0.1 uM, or 0 uM drug (caffeine for group F, nicotine for group G, or CBD for group H).

Day 5, flow cytometry was performed, and the cells were gate on GFP/BFP

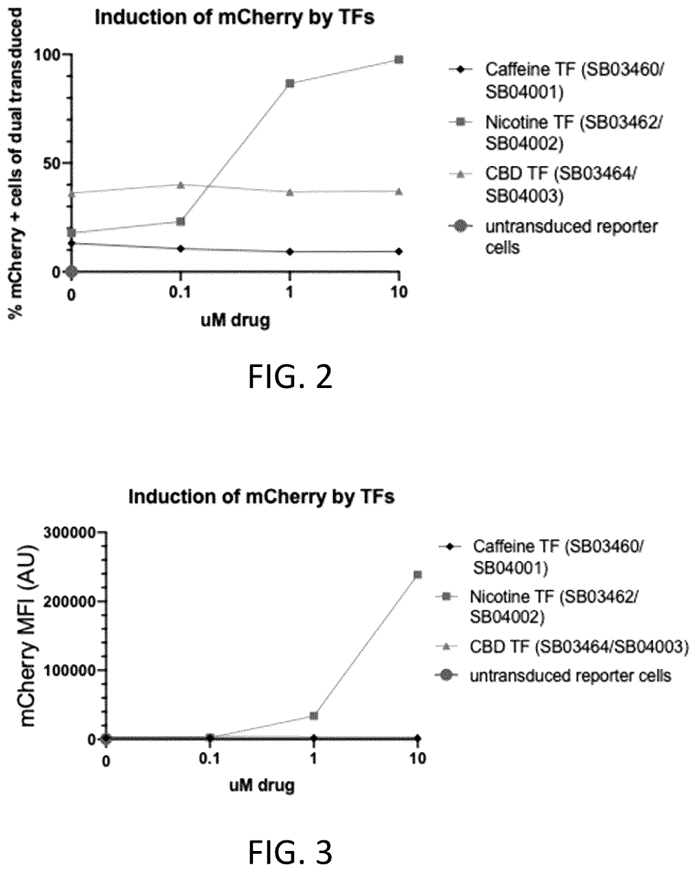

positive cells and assessed for mCherry expression. mCherry expression was analyzed and plotted as a percent of mCherry positive cells (FIG. 2), and by mean fluorescent intensity of mCherry (FIG. 3).

### Results

As shown in FIG. 2 and FIG. 3, the nicotine treatment at concentrations of 10 uM and 1 uM induced concentration-dependent reporter expression in cells expressing a nicotine-inducible transcription factor including an NLS in the first monomer and in the second monomer.

### Example 3: Transcription Modulation by Drug-Induced Transcription Factors

Transduction assays were performed to assess mCherry expression in response to inducible transcription factors activated by mifepristone.

### Materials and Methods

U87-MG cells were transduced with a lentivirus encoding a synthetic ITF-responsive promoter (SEQ ID NO: 65) that includes 4×ZF binding sites that bind to ZF10 linked to YBTATA, a minimal promoter. The promoter drives expression of mCherry. This cell line was called “reporter cells.” The mifepristone-inducible transcription factor construct includes (from N-terminus to C-terminus) a zinc finger protein domain (“ZF10”) of SEQ ID NO: 1, a progesterone receptor domain of SEQ ID NO: 68, a p65 transcriptional activator domain of SEQ ID NO:70, a cleavable linker (E2A-T2A) of SEQ ID NO:72, and a blue fluorescent protein tag (BFP) of SEQ ID NO:74. On day 1, reporter cells were transduced with lentivirus encoding the mifepristone-inducible transcription factor construct.

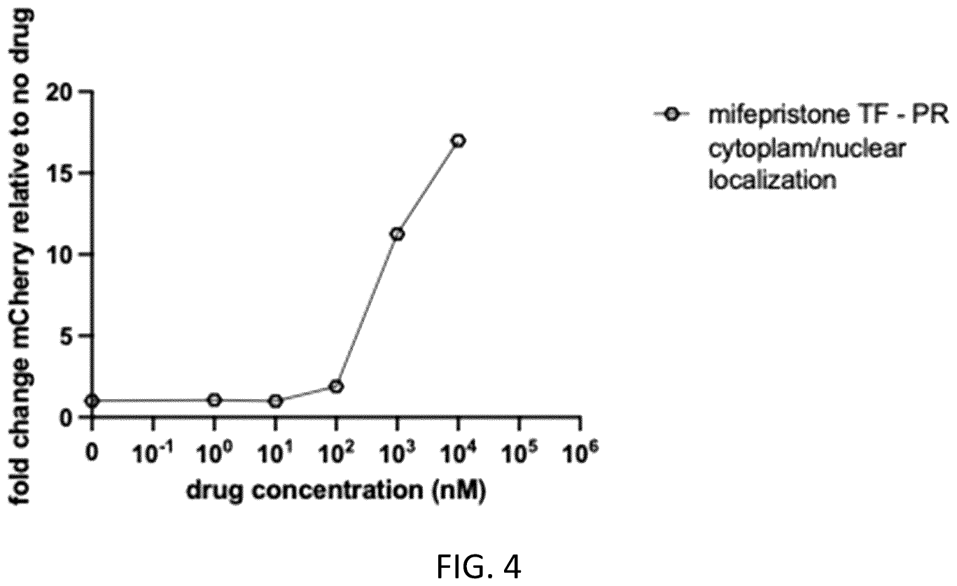

To assay for mifepristone-regulated transcription modulation, cell group E was thawed and four days later, the cells were split into a 48-well plate and treated with 0 nM, 1 nM, 100 nM, 1000 nM, or 10000 nM mifepristone. Six days after the cells were thawed, cells were trypsinized and then resuspended in FACS buffer plus Sytox Red (fluoresces in APC channel) viability dye. Cell mCherry geometric mean fluorescent intensity (gMFI) was measured at each concentration and normalized to no drug as shown in FIG. 4.

### Results

As shown in FIG. 4, the mifepristone-regulated transcription modulation system induced mCherry expression in the presence of drug in a concentration-dependent manner.

TABLE 14
Sequences

| Name | SEQ ID NO | Sequence |
|---|---|---|
| ZF Protein | 1 | SRPGERPFQCRICMRNFSRRHGLDRHTRTHTGEKPFQCRICMR |
| Domain | | NFSDHSSLKRHLRTHTGSQKPFQCRICMRNFSVRHNLTRHLRT |
| (ZF10) | | HTGEKPFQCRICMRNFSDHSNLSRHLKTHTGSQKPFQCRICMR |
| | | NFSQRSSLVRHLRTHTGEKPFQCRICMRNFSESGHLKRHLRTHL |
| | | RGS |
| NLS | 2 | PKKKRKVG |
| NLS | 3 | KRPAATKKRGQRKKKK |
| NLS | 4 | AAFEDLRVLS |
| NES | 5 | DNGMEELSQALASSFSVS |
| NES | 6 | MEELSQALASSFSV |
| NES | 7 | PLQLPPLERLTL |
| NES | 8 | NELALKLAGLDI |
| NES | 9 | ERFEMFRELNEALEL |
| NES | 10 | DHAEKVAEKLEALSV |
| NES | 11 | QLVEELLKIICAFQL |
| NES | 12 | TNLEALQKKLEELEL |
| NES | 13 | DVKEEMTSALATMRV |
| NES | 14 | STNGSLAAEFRHLQL |
| Peptide | 15 | GSGSGSGS |
| Linker | | |
| Peptide | 16 | KEGS |
| Linker | | |
| Peptide | 17 | EAAAK |
| Linker | | |
| Peptide | 18 | AAPAKQE |
| Linker | | |
| Peptide | 19 | GSGSGSGSGGAEAAAKEAAAKEAAAKA |
| Linker | | |
| Peptide | 20 | AAPAKQEAAAPAKQEAAAPAKQEAAAPAPAAKAEAPAAAPA |
| Linker | | AKA |
| Peptide | 21 | AEAAAKEAAAKEAAAKA |
| Linker | | |
| minimal | 22 | AGAGGGTATATAATGGAAGCTCGACTTCCAG |
| promoter; | | |
| minP | | |
| NFKB | 23 | GGGAATTTCCGGGGACTTTCCGGGAATTTCCGGGGACTTTCC |
| response | | GGGAATTTCC |
| element | | |
| protein | | |
| promoter; 5x | | |
| NFKB-RE | | |
| CREB | 24 | CACCAGACAGTGACGTCAGCTGCCAGATCCCATGGCCGTCA |
| response | | TACTGTGACGTCTTTCAGACACCCCATTGACGTCAATGGGAG |
| element | | AA |
| protein | | |
| promoter; 4x | | |
| CRE | | |
| NFAT | 25 | GGAGGAAAAACTGTTTCATACAGAAGGCGTGGAGGAAAAA |
| response | | CTGTTTCATACAGAAGGCGTGGAGGAAAAACTGTTTCATAC |
| element | | AGAAGGCGT |
| protein | | |
| promoter; 3x | | |
| NFAT | | |
| binding sites | | |
| SRF response | 26 | AGGATGTCCATATTAGGACATCTAGGATGTCCATATTAGGA |
| element | | CATCTAGGATGTCCATATTAGGACATCTAGGATGTCCATATT |
| protein | | AGGACATCTAGGATGTCCATATTAGGACATCT |
| promoter; 5x | | |
| SRE | | |
| SRF response | 27 | AGTATGTCCATATTAGGACATCTACCATGTCCATATTAGGAC |
| element | | ATCTACTATGTCCATATTAGGACATCTTGTATGTCCATATTA |
| protein | | GGACATCTAAAATGTCCATATTAGGACATCT |
| promoter 2; | | |
| 5x SRF-RE | | |
| AP1 response | 28 | TGAGTCAGTGACTCAGTGAGTCAGTGACTCAGTGAGTCAGT |
| element | | GACTCAG |
| protein | | |
| promoter; 6x | | |
| AP1-RE | | |
| TCF-LEF | 29 | AGATCAAAGGGTTTAAGATCAAAGGGCTTAAGATCAAAGGG |
| response | | TATAAGATCAAAGGGCCTAAGATCAAAGGGACTAAGATCAA |
| element | | AGGGTTTAAGATCAAAGGGCTTAAGATCAAAGGGCCTA |
| promoter; 8x | | |
| TCF-LEF-RE | | |
| SBEx4 | 30 | GTCTAGACGTCTAGACGTCTAGACGTCTAGAC |
| SMAD2/3 - | 31 | CAGACACAGACACAGACACAGACA |
| CAGACA x4 | | |
| STAT3 | 32 | Ggatccggtactcgagatctgcgatctaagtaagcttggcattccggtactgttggtaaagccac |
| binding site | | |
| CMV | 33 | GTTGACATTGATTATTGACTAGTTATTAATAGTAATCAATTA |
| | | CGGGGTCATTAGTTCATAGCCCATATATGGAGTTCCGCGTTA |
| | | CATAACTTACGGTAAATGGCCCGCCTGGCTGACCGCCCAAC |
| | | GACCCCCGCCCATTGACGTCAATAATGACGTATGTTCCCATA |
| | | GTAACGCCAATAGGGACTTTCCATTGACGTCAATGGGTGGA |
| | | GTATTTACGGTAAACTGCCCACTTGGCAGTACATCAAGTGTA |
| | | TCATATGCCAAGTACGCCCCCTATTGACGTCAATGACGGTA |
| | | AATGGCCCGCCTGGCATTATGCCCAGTACATGACCTTATGG |
| | | GACTTTCCTACTTGGCAGTACATCTACGTATTAGTCATCGCT |
| | | ATTACCATGGTGATGCGGTTTTGGCAGTACATCAATGGGCGT |
| | | GGATAGCGGTTTGACTCACGGGGATTTCCAAGTCTCCACCC |
| | | CATTGACGTCAATGGGAGTTTGTTTTGGCACCAAAATCAAC |
| | | GGGACTTTCCAAAATGTCGTAACAACTCCGCCCCATTGACG |
| | | CAAATGGGCGGTAGGCGTGTACGGTGGGAGGTCTATATAAG |
| | | CAGAGCTC |
| EF1a | 34 | GGCTCCGGTGCCCGTCAGTGGGCAGAGCGCACATCGCCCAC |
| | | AGTCCCCGAGAAGTTGGGGGGAGGGGTCGGCAATTGAACCG |
| | | GTGCCTAGAGAAGGTGGCGCGGGGTAAACTGGGAAAGTGA |
| | | TGCCGTGTACTGGCTCCGCCTTTTTCCCGAGGGTGGGGGAGA |
| | | ACCGTATATAAGTGCAGTAGTCGCCGTGAACGTTCTTTTTCG |
| | | CAACGGGTTTGCCGCCAGAACACAGGTAAGTGCCGTGTGTG |
| | | GTTCCCGCGGGCCTGGCCTCTTTACGGGTTATGGCCCTTGCG |
| | | TGCCTTGAATTACTTCCACCTGGCTGCAGTACGTGATTCTTG |
| | | ATCCCGAGCTTCGGGTTGGAAGTGGGTGGGAGAGTTCGAGG |
| | | CCTTGCGCTTAAGGAGCCCCTTCGCCTCGTGCTTGAGTTGAG |
| | | GCCTGGCCTGGGCGCTGGGGCCGCCGCGTGCGAATCTGGTG |
| | | GCACCTTCGCGCCTGTCTCGCTGCTTTCGATAAGTCTCTAGC |
| | | CATTTAAAATTTTTGATGACCTGCTGCGACGCTTTTTTTCTGG |
| | | CAAGATAGTCTTGTAAATGCGGGCCAAGATCTGCACACTGG |
| | | TATTTCGGTTTTTGGGGCCGCGGGCGGCGACGGGGCCCGTG |
| | | CGTCCCAGCGCACATGTTCGGCGAGGCGGGGCCTGCGAGCG |
| | | CGACCACCGAGAATCGGACGGGGGTAGTCTCAAGCTGGCCG |
| | | GCCTGCTCTGGTGCCTGTCCTCGCGCCGCCGTGTATCGCCCC |
| | | GCCCCGGGCGGCAAGGCTGGCCCGGTCGGCACCAGTTGCGT |
| | | GAGCGGAAAGATGGCCGCTTCCCGGTCCTGCTGCAGGGAGC |
| | | TCAAAATGGAGGACGCGGCGCTCGGGAGAGCGGGCGGGTG |
| | | AGTCACCCACACAAAGGAAAAGGGCCTTTCCGTCCTCAGCC |
| | | GTCGCTTCATGTGACTCCACGGAGTACCGGGCGCCGTCCAG |
| | | GCACCTCGATTAGTTCTCGAGCTTTTGGAGTACGTCGTCTTT |
| | | AGGTTGGGGGGAGGGGTTTTATGCGATGGAGTTTCCCCACA |
| | | CTGAGTGGGTGGAGACTGAAGTTAGGCCAGCTTGGCACTTG |
| | | ATGTAATTCTCCTTGGAATTTGCCCTTTTTGAGTTTGGATCTT |
| | | GGTTCATTCTCAAGCCTCAGACAGTGGTTCAAAGTTTTTTTC |
| | | TTCCATTTCAGGTGTCGTGA |
| EFS | 35 | GGATCTGCGATCGCTCCGGTGCCCGTCAGTGGGCAGAGCGC |
| | | ACATCGCCCACAGTCCCCGAGAAGTTGGGGGGAGGGGTCGG |
| | | CAATTGAACCGGTGCCTAGAGAAGGTGGCGCGGGGTAAACT |
| | | GGGAAAGTGATGTCGTGTACTGGCTCCGCCTTTTTCCCGAGG |
| | | GTGGGGGAGAACCGTATATAAGTGCAGTAGTCGCCGTGAAC |
| | | GTTCTTTTTCGCAACGGGTTTGCCGCCAGAACACAGCTGAAG |
| | | CTTCGAGGGGCTCGCATCTCTCCTTCACGCGCCCGCCGCCCT |
| | | ACCTGAGGCCGCCATCCACGCCGGTTGAGTCGCGTTCTGCC |
| | | GCCTCCCGCCTGTGGTGCCTCCTGAACTGCGTCCGCCGTCTA |
| | | GGTAAGTTTAAAGCTCAGGTCGAGACCGGGCCTTTGTCCGG |
| | | CGCTCCCTTGGAGCCTACCTAGACTCAGCCGGCTCTCCACGC |
| | | TTTGCCTGACCCTGCTTGCTCAACTCTACGTCTTTGTTTCGTT |
| | | TTCTGTTCTGCGCCGTTACAGATCCAAGCTGTGACCGGCGCC |
| | | TAC |
| MND | 36 | TTTATTTAGTCTCCAGAAAAAGGGGGGAATGAAAGACCCCA |
| | | CCTGTAGGTTTGGCAAGCTAGGATCAAGGTTAGGAACAGAG |
| | | AGACAGCAGAATATGGGCCAAACAGGATATCTGTGGTAAGC |
| | | AGTTCCTGCCCCGGCTCAGGGCCAAGAACAGTTGGAACAGC |
| | | AGAATATGGGCCAAACAGGATATCTGTGGTAAGCAGTTCCT |
| | | GCCCCGGCTCAGGGCCAAGAACAGATGGTCCCCAGATGCGG |
| | | TCCCGCCCTCAGCAGTTTCTAGAGAACCATCAGATGTTTCCA |
| | | GGGTGCCCCAAGGACCTGAAATGACCCTGTGCCTTATTTGA |
| | | ACTAACCAATCAGTTCGCTTCTCGCTTCTGTTCGCGCGCTTC |
| | | TGCTCCCCGAGCTCAATAAAAGAGCCCA |
| PGK | 37 | GGGGTTGGGGTTGCGCCTTTTCCAAGGCAGCCCTGGGTTTGC |
| | | GCAGGGACGCGGCTGCTCTGGGCGTGGTTCCGGGAAACGCA |
| | | GCGGCGCCGACCCTGGGTCTCGCACATTCTTCACGTCCGTTC |
| | | GCAGCGTCACCCGGATCTTCGCCGCTACCCTTGTGGGCCCCC |
| | | CGGCGACGCTTCCTGCTCCGCCCCTAAGTCGGGAAGGTTCCT |
| | | TGCGGTTCGCGGCGTGCCGGACGTGACAAACGGAAGCCGCA |
| | | CGTCTCACTAGTACCCTCGCAGACGGACAGCGCCAGGGAGC |
| | | AATGGCAGCGCGCCGACCGCGATGGGCTGTGGCCAATAGCG |
| | | GCTGCTCAGCGGGGCGCGCCGAGAGCAGCGGCCGGGAAGG |
| | | GGCGGTGCGGGAGGCGGGGTGTGGGGCGGTAGTGTGGGCC |
| | | CTGTTCCTGCCCGCGCGGTGTTCCGCATTCTGCAAGCCTCCG |
| | | GAGCGCACGTCGGCAGTCGGCTCCCTCGTTGACCGAATCAC |
| | | CGACCTCTCTCCCCAG |
| SFFV | 38 | GTAACGCCATTTTGCAAGGCATGGAAAAATACCAAACCAAG |
| | | AATAGAGAAGTTCAGATCAAGGGCGGGTACATGAAAATAG |
| | | CTAACGTTGGGCCAAACAGGATATCTGCGGTGAGCAGTTTC |
| | | GGCCCCGGCCCGGGGCCAAGAACAGATGGTCACCGCAGTTT |
| | | CGGCCCCGGCCCGAGGCCAAGAACAGATGGTCCCCAGATAT |
| | | GGCCCAACCCTCAGCAGTTTCTTAAGACCCATCAGATGTTTC |
| | | CAGGCTCCCCCAAGGACCTGAAATGACCCTGCGCCTTATTT |
| | | GAATTAACCAATCAGCCTGCTTCTCGCTTCTGTTCGCGCGCT |
| | | TCTGCTTCCCGAGCTCTATAAAAGAGCTCACAACCCCTCACT |
| | | CGGCGCGCCAGTCCTCCGACAGACTGAGTCGCCCGGG |
| SV40 | 39 | CTGTGGAATGTGTGTCAGTTAGGGTGTGGAAAGTCCCCAGG |
| | | CTCCCCAGCAGGCAGAAGTATGCAAAGCATGCATCTCAATT |
| | | AGTCAGCAACCAGGTGTGGAAAGTCCCCAGGCTCCCCAGCA |
| | | GGCAGAAGTATGCAAAGCATGCATCTCAATTAGTCAGCAAC |
| | | CATAGTCCCGCCCCTAACTCCGCCCATCCCGCCCCTAACTCC |
| | | GCCCAGTTCCGCCCATTCTCCGCCCCATGGCTGACTAATTTT |
| | | TTTTATTTATGCAGAGGCCGAGGCCGCCTCTGCCTCTGAGCT |
| | | ATTCCAGAAGTAGTGAGGAGGCTTTTTTGGAGGCCTAGGCT |
| | | TTTGCAAAAAGCT |
| UbC | 40 | GCGCCGGGTTTTGGCGCCTCCCGCGGGCGCCCCCCTCCTCAC |
| | | GGCGAGCGCTGCCACGTCAGACGAAGGGCGCAGGAGCGTT |
| | | CCTGATCCTTCCGCCCGGACGCTCAGGACAGCGGCCCGCTG |
| | | CTCATAAGACTCGGCCTTAGAACCCCAGTATCAGCAGAAGG |
| | | ACATTTTAGGACGGGACTTGGGTGACTCTAGGGCACTGGTTT |
| | | TCTTTCCAGAGAGCGGAACAGGCGAGGAAAAGTAGTCCCTT |
| | | CTCGGCGATTCTGCGGAGGGATCTCCGTGGGGCGGTGAACG |
| | | CCGATGATTATATAAGGACGCGCCGGGTGTGGCACAGCTAG |
| | | TTCCGTCGCAGCCGGGATTTGGGTCGCGGTTCTTGTTTGTGG |
| | | ATCGCTGTGATCGTCACTTGGTGAGTTGCGGGCTGCTGGGCT |
| | | GGCCGGGGCTTTCGTGGCCGCCGGGCCGCTCGGTGGGACGG |
| | | AAGCGTGTGGAGAGACCGCCAAGGGCTGTAGTCTGGGTCCG |
| | | CGAGCAAGGTTGCCCTGAACTGGGGGTTGGGGGGAGCGCAC |
| | | AAAATGGCGGCTGTTCCCGAGTCTTGAATGGAAGACGCTTG |
| | | TAAGGCGGGCTGTGAGGTCGTTGAAACAAGGTGGGGGGCAT |
| | | GGTGGGCGGCAAGAACCCAAGGTCTTGAGGCCTTCGCTAAT |
| | | GCGGGAAAGCTCTTATTCGGGTGAGATGGGCTGGGGCACCA |
| | | TCTGGGGACCCTGACGTGAAGTTTGTCACTGACTGGAGAAC |
| | | TCGGGTTTGTCGTCTGGTTGCGGGGGCGGCAGTTATGCGGTG |
| | | CCGTTGGGCAGTGCACCCGTACCTTTGGGAGCGCGCGCCTC |
| | | GTCGTGTCGTGACGTCACCCGTTCTGTTGGCTTATAATGCAG |
| | | GGTGGGGCCACCTGCCGGTAGGTGTGCGGTAGGCTTTTCTCC |
| | | GTCGCAGGACGCAGGGTTCGGGCCTAGGGTAGGCTCTCCTG |
| | | AATCGACAGGCGCCGGACCTCTGGTGAGGGGAGGGATAAGT |
| | | GAGGCGTCAGTTTCTTTGGTCGGTTTTATGTACCTATCTTCTT |
| | | AAGTAGCTGAAGCTCCGGTTTTGAACTATGCGCTCGGGGTT |
| | | GGCGAGTGTGTTTTGTGAAGTTTTTTAGGCACCTTTTGAAAT |
| | | GTAATCATTTGGGTCAATATGTAATTTTCAGTGTTAGACTAG |
| | | TAAAGCTTCTGCAGGTCGACTCTAGAAAATTGTCCGCTAAAT |
| | | TCTGGCCGTTTTTGGCTTTTTTGTTAGAC |
| hEF1aV1 | 41 | GGCTCCGGTGCCCGTCAGTGGGCAGAGCGCACATCGCCCAC |
| | | AGTCCCCGAGAAGTTGGGGGGAGGGGTCGGCAATTGAACCG |
| | | GTGCCTAGAGAAGGTGGCGCGGGGTAAACTGGGAAAGTGA |
| | | TGTCGTGTACTGGCTCCGCCTTTTTCCCGAGGGTGGGGGAGA |
| | | ACCGTATATAAGTGCAGTAGTCGCCGTGAACGTTCTTTTTCG |
| | | CAACGGGTTTGCCGCCAGAACACAGGTAAGTGCCGTGTGTG |
| | | GTTCCCGCGGGCCTGGCCTCTTTACGGGTTATGGCCCTTGCG |
| | | TGCCTTGAATTACTTCCACCTGGCTGCAGTACGTGATTCTTG |
| | | ATCCCGAGCTTCGGGTTGGAAGTGGGTGGGAGAGTTCGAGG |
| | | CCTTGCGCTTAAGGAGCCCCTTCGCCTCGTGCTTGAGTTGAG |
| | | GCCTGGCCTGGGCGCTGGGGCCGCCGCGTGCGAATCTGGTG |
| | | GCACCTTCGCGCCTGTCTCGCTGCTTTCGATAAGTCTCTAGC |
| | | CATTTAAAATTTTTGATGACCTGCTGCGACGCTTTTTTTCTGG |
| | | CAAGATAGTCTTGTAAATGCGGGCCAAGATCTGCACACTGG |
| | | TATTTCGGTTTTTGGGGCCGCGGGCGGCGACGGGGCCCGTG |
| | | CGTCCCAGCGCACATGTTCGGCGAGGCGGGGCCTGCGAGCG |
| | | CGGCCACCGAGAATCGGACGGGGGTAGTCTCAAGCTGGCCG |
| | | GCCTGCTCTGGTGCCTGGTCTCGCGCCGCCGTGTATCGCCCC |
| | | GCCCTGGGCGGCAAGGCTGGCCCGGTCGGCACCAGTTGCGT |
| | | GAGCGGAAAGATGGCCGCTTCCCGGCCCTGCTGCAGGGAGC |
| | | TCAAAATGGAGGACGCGGCGCTCGGGAGAGCGGGGGGTG |
| | | AGTCACCCACACAAAGGAAAAGGGCCTTTCCGTCCTCAGCC |
| | | GTCGCTTCATGTGACTCCACGGAGTACCGGGCGCCGTCCAG |
| | | GCACCTCGATTAGTTCTCGAGCTTTTGGAGTACGTCGTCTTT |
| | | AGGTTGGGGGGAGGGGTTTTATGCGATGGAGTTTCCCCACA |
| | | CTGAGTGGGTGGAGACTGAAGTTAGGCCAGCTTGGCACTTG |
| | | ATGTAATTCTCCTTGGAATTTGCCCTTTTTGAGTTTGGATCTT |
| | | GGTTCATTCTCAAGCCTCAGACAGTGGTTCAAAGTTTTTTTC |
| | | TTCCATTTCAGGTGTCGTGA |
| hCAGG | 42 | ACTAGTTATTAATAGTAATCAATTACGGGGTCATTAGTTCAT |
| | | AGCCCATATATGGAGTTCCGCGTTACATAACTTACGGTAAAT |
| | | GGCCCGCCTGGCTGACCGCCCAACGACCCCCGCCCATTGAC |
| | | GTCAATAATGACGTATGTTCCCATAGTAACGCCAATAGGGA |
| | | CTTTCCATTGACGTCAATGGGTGGAGTATTTACGGTAAACTG |
| | | CCCACTTGGCAGTACATCAAGTGTATCATATGCCAAGTACG |
| | | CCCCCTATTGACGTCAATGACGGTAAATGGCCCGCCTGGCA |
| | | TTATGCCCAGTACATGACCTTATGGGACTTTCCTACTTGGCA |
| | | GTACATCTACGTATTAGTCATCGCTATTACCATGGTCGAGGT |
| | | GAGCCCCACGTTCTGCTTCACTCTCCCCATCTCCCCCCCCTC |
| | | CCCACCCCCAATTTTGTATTTATTTATTTTTTAATTATTTTGT |
| | | GCAGCGATGGGGGCGGGGGGGGGGGGGGGGCGCGCGCCAG |
| | | GCGGGGGGGGCGGGGCGAGGGGCGGGGCGGGGCGAGGCG |
| | | GAGAGGTGCGGCGGCAGCCAATCAGAGCGGCGCGCTCCGA |
| | | AAGTTTCCTTTTATGGCGAGGCGGCGGCGGCGGCGGCCCTA |
| | | TAAAAAGCGAAGCGCGCGGCGGGCGGGGAGTCGCTGCGAC |
| | | GCTGCCTTCGCCCCGTGCCCCGCTCCGCCGCCGCCTCGCGCC |
| | | GCCCGCCCCGGCTCTGACTGACCGCGTTACTCCCACAGGTG |
| | | AGCGGGCGGGACGGCCCTTCTCCTCCGGGCTGTAATTAGCG |
| | | CTTGGTTTAATGACGGCTTGTTTCTTTTCTGTGGCTGCGTGA |
| | | AAGCCTTGAGGGGCTCCGGGAGGGCCCTTTGTGCGGGGGGA |
| | | GCGGCTCGGGGGGTGCGTGCGTGTGTGTGTGCGTGGGGAGC |
| | | GCCGCGTGCGGCTCCGCGCTGCCCGGCGGCTGTGAGCGCTG |
| | | CGGGCGCGGCGCGGGGCTTTGTGCGCTCCGCAGTGTGCGCG |
| | | AGGGGAGCGCGGCCGGGGGCGGTGCCCCGCGGTGCGGGGG |
| | | GGGCTGCGAGGGGAACAAAGGCTGCGTGCGGGGTGTGTGC |
| | | GTGGGGGGGTGAGCAGGGGGTGTGGGCGCGTCGGTCGGGCT |
| | | GCAACCCCCCCTGCACCCCCCTCCCCGAGTTGCTGAGCACG |
| | | GCCCGGCTTCGGGTGCGGGGCTCCGTACGGGGCGTGGCGCG |
| | | GGGCTCGCCGTGCCGGGCGGGGGGTGGCGGCAGGTGGGGG |
| | | TGCCGGGCGGGGCGGGGCCGCCTCGGGCCGGGGAGGGCTC |
| | | GGGGGAGGGGCGCGGCGGCCCCCGGAGCGCCGGCGGCTGT |
| | | CGAGGCGCGGCGAGCCGCAGCCATTGCCTTTTATGGTAATC |
| | | GTGCGAGAGGGCGCAGGGACTTCCTTTGTCCCAAATCTGTG |
| | | CGGAGCCGAAATCTGGGAGGCGCCGCCGCACCCCCTCTAGC |
| | | GGGCGCGGGGCGAAGCGGTGCGGCGCCGGCAGGAAGGAAA |
| | | TGGGCGGGGAGGGCCTTCGTGCGTCGCCGCGCCGCCGTCCC |
| | | CTTCTCCCTCTCCAGCCTCGGGGCTGTCCGCGGGGGGACGG |
| | | CTGCCTTCGGGGGGGACGGGGCAGGGCGGGGTTCGGCTTCT |
| | | GGCGTGTGACCGGCGGCTCTAGAGCCTCTGCTAACCATGTT |
| | | CATGCCTTCTTCTTTTTCCTACAGCTCCTGGGCAACGTGCTG |
| | | GTTATTGTGCTGTCTCATCATTTTGGCAAAGAATTC |
| hEF1aV2 | 43 | GGGCAGAGCGCACATCGCCCACAGTCCCCGAGAAGTTGGGG |
| | | GGAGGGGTCGGCAATTGAACCGGTGCCTAGAGAAGGTGGC |
| | | GCGGGGTAAACTGGGAAAGTGATGTCGTGTACTGGCTCCGC |
| | | CTTTTTCCCGAGGGTGGGGGAGAACCGTATATAAGTGCAGT |
| | | AGTCGCCGTGAACGTTCTTTTTCGCAACGGGTTTGCCGCCAG |
| | | AACACAG |
| hACTb | 44 | CCACTAGTTCCATGTCCTTATATGGACTCATCTTTGCCTATTG |
| | | CGACACACACTCAATGAACACCTACTACGCGCTGCAAAGAG |
| | | CCCCGCAGGCCTGAGGTGCCCCCACCTCACCACTCTTCCTAT |
| | | TTTTGTGTAAAAATCCAGCTTCTTGTCACCACCTCCAAGGAG |
| | | GGGGAGGAGGAGGAAGGCAGGTTCCTCTAGGCTGAGCCGA |
| | | ATGCCCCTCTGTGGTCCCACGCCACTGATCGCTGCATGCCCA |
| | | CCACCTGGGTACACACAGTCTGTGATTCCCGGAGCAGAACG |
| | | GACCCTGCCCACCCGGTCTTGTGTGCTACTCAGTGGACAGA |
| | | CCCAAGGCAAGAAAGGGTGACAAGGACAGGGTCTTCCCAG |
| | | GCTGGCTTTGAGTTCCTAGCACCGCCCCGCCCCCAATCCTCT |
| | | GTGGCACATGGAGTCTTGGTCCCCAGAGTCCCCCAGCGGCC |
| | | TCCAGATGGTCTGGGAGGGCAGTTCAGCTGTGGCTGCGCAT |
| | | AGCAGACATACAACGGACGGTGGGCCCAGACCCAGGCTGT |
| | | GTAGACCCAGCCCCCCCGCCCCGCAGTGCCTAGGTCACCCA |
| | | CTAACGCCCCAGGCCTGGTCTTGGCTGGGCGTGACTGTTACC |
| | | CTCAAAAGCAGGCAGCTCCAGGGTAAAAGGTGCCCTGCCCT |
| | | GTAGAGCCCACCTTCCTTCCCAGGGCTGCGGCTGGGTAGGT |
| | | TTGTAGCCTTCATCACGGGCCACCTCCAGCCACTGGACCGCT |
| | | GGCCCCTGCCCTGTCCTGGGGAGTGTGGTCCTGCGACTTCTA |
| | | AGTGGCCGCAAGCCACCTGACTCCCCCAACACCACACTCTA |
| | | CCTCTCAAGCCCAGGTCTCTCCCTAGTGACCCACCCAGCACA |
| | | TTTAGCTAGCTGAGCCCCACAGCCAGAGGTCCTCAGGCCCT |
| | | GCTTTCAGGGCAGTTGCTCTGAAGTCGGCAAGGGGGAGTGA |
| | | CTGCCTGGCCACTCCATGCCCTCCAAGAGCTCCTTCTGCAGG |
| | | AGCGTACAGAACCCAGGGCCCTGGCACCCGTGCAGACCCTG |
| | | GCCCACCCCACCTGGGCGCTCAGTGCCCAAGAGATGTCCAC |
| | | ACCTAGGATGTCCCGCGGTGGGTGGGGGGCCCGAGAGACG |
| | | GGCAGGCCGGGGGCAGGCCTGGCCATGCGGGGCCGAACCG |
| | | GGCACTGCCCAGCGTGGGGCGCGGGGGCCACGGCGCGCGC |
| | | CCCCAGCCCCCGGGCCCAGCACCCCAAGGCGGCCAACGCCA |
| | | AAACTCTCCCTCCTCCTCTTCCTCAATCTCGCTCTCGCTCTTT |
| | | TTTTTTTTCGCAAAAGGAGGGGAGAGGGGGTAAAAAAATGC |
| | | TGCACTGTGCGGCGAAGCCGGTGAGTGAGCGGCGCGGGGCC |
| | | AATCAGCGTGCGCCGTTCCGAAAGTTGCCTTTTATGGCTCGA |
| | | GCGGCCGCGGCGGCGCCCTATAAAACCCAGCGGCGCGACGC |
| | | GCCACCACCGCCGAGACCGCGTCCGCCCCGCGAGCACAGAG |
| | | CCTCGCCTTTGCCGATCCGCCGCCCGTCCACACCCGCCGCCA |
| | | GGTAAGCCCGGCCAGCCGACCGGGGCAGGCGGCTCACGGC |
| | | CCGGCCGCAGGCGGCCGCGGCCCCTTCGCCCGTGCAGAGCC |
| | | GCCGTCTGGGCCGCAGCGGGGGGCGCATGGGGGGGGAACC |
| | | GGACCGCCGTGGGGGGCGCGGGAGAAGCCCCTGGGCCTCC |
| | | GGAGATGGGGGACACCCCACGCCAGTTCGGAGGCGCGAGG |
| | | CCGCGCTCGGGAGGCGCGCTCCGGGGGTGCCGCTCTCGGGG |
| | | CGGGGGCAACCGGCGGGGTCTTTGTCTGAGCCGGGCTCTTG |
| | | CCAATGGGGATCGCAGGGTGGGCGCGGCGGAGCCCCCGCC |
| | | AGGCCCGGTGGGGGCTGGGGCGCCATTGCGCGTGCGCGCTG |
| | | GTCCTTTGGGCGCTAACTGCGTGCGCGCTGGGAATTGGCGCT |
| | | AATTGCGCGTGCGCGCTGGGACTCAAGGCGCTAACTGCGCG |
| | | TGCGTTCTGGGGCCCGGGGTGCCGCGGCCTGGGCTGGGGCG |
| | | AAGGCGGGCTCGGCCGGAAGGGGTGGGGTCGCCGCGGCTC |
| | | CCGGGCGCTTGCGCGCACTTCCTGCCCGAGCCGCTGGCCGC |
| | | CCGAGGGTGTGGCCGCTGCGTGCGCGCGCGCCGACCCGGCG |
| | | CTGTTTGAACCGGGCGGAGGCGGGGCTGGCGCCCGGTTGGG |
| | | AGGGGGTTGGGGCCTGGCTTCCTGCCGCGCGCCGCGGGGAC |
| | | GCCTCCGACCAGTGTTTGCCTTTTATGGTAATAACGCGGCCG |
| | | GCCCGGCTTCCTTTGTCCCCAATCTGGGCGCGCGCCGGCGCC |
| | | CCCTGGCGGCCTAAGGACTCGGCGCGCCGGAAGTGGCCAGG |
| | | GCGGGGGCGACCTCGGCTCACAGCGCGCCCGGCTAT |
| heIF4A1 | 45 | GTTGATTTCCTTCATCCCTGGCACACGTCCAGGCAGTGTCGA |
| | | ATCCATCTCTGCTACAGGGGAAAACAAATAACATTTGAGTC |
| | | CAGTGGAGACCGGGAGCAGAAGTAAAGGGAAGTGATAACC |
| | | CCCAGAGCCCGGAAGCCTCTGGAGGCTGAGACCTCGCCCCC |
| | | CTTGCGTGATAGGGCCTACGGAGCCACATGACCAAGGCACT |
| | | GTCGCCTCCGCACGTGTGAGAGTGCAGGGCCCCAAGATGGC |
| | | TGCCAGGCCTCGAGGCCTGACTCTTCTATGTCACTTCCGTAC |
| | | CGGCGAGAAAGGCGGGCCCTCCAGCCAATGAGGCTGCGGG |
| | | GCGGGCCTTCACCTTGATAGGCACTCGAGTTATCCAATGGTG |
| | | CCTGCGGGCCGGAGCGACTAGGAACTAACGTCATGCCGAGT |
| | | TGCTGAGCGCCGGCAGGCGGGGCCGGGGCGGCCAAACCAA |
| | | TGCGATGGCCGGGGCGGAGTCGGGCGCTCTATAAGTTGTCG |
| | | ATAGGCGGGCACTCCGCCCTAGTTTCTAAGGACCATG |
| hGAPDH | 46 | AGTTCCCCAACTTTCCCGCCTCTCAGCCTTTGAAAGAAAGAA |
| | | AGGGGAGGGGGCAGGCCGCGTGCAGTCGCGAGCGGTGCTG |
| | | GGCTCCGGCTCCAATTCCCCATCTCAGTCGCTCCCAAAGTCC |
| | | TTCTGTTTCATCCAAGCGTGTAAGGGTCCCCGTCCTTGACTC |
| | | CCTAGTGTCCTGCTGCCCACAGTCCAGTCCTGGGAACCAGC |
| | | ACCGATCACCTCCCATCGGGCCAATCTCAGTCCCTTCCCCCC |
| | | TACGTCGGGGCCCACACGCTCGGTGCGTGCCCAGTTGAACC |
| | | AGGCGGCTGCGGAAAAAAAAAAGCGGGGAGAAAGTAGGGC |
| | | CCGGCTACTAGCGGTTTTACGGGCGCACGTAGCTCAGGCCT |
| | | CAAGACCTTGGGCTGGGACTGGCTGAGCCTGGCGGGAGGCG |
| | | GGGTCCGAGTCACCGCCTGCCGCCGCGCCCCCGGTTTCTATA |
| | | AATTGAGCCCGCAGCCTCCCGCTTCGCTCTCTGCTCCTCCTG |
| | | TTCGACAGTCAGCCGCATCTTCTTTTGCGTCGCCAGGTGAAG |
| | | ACGGGCGGAGAGAAACCCGGGAGGCTAGGGACGGCCTGAA |
| | | GGCGGCAGGGGGGGGCGCAGGCCGGATGTGTTCGCGCCGCT |
| | | GCGGGGTGGGCCCGGGCGGCCTCCGCATTGCAGGGGGGGGC |
| | | GGAGGACGTGATGCGGCGCGGGCTGGGCATGGAGGCCTGGT |
| | | GGGGGAGGGGAGGGGAGGCGTGGGTGTCGGCCGGGGCCAC |
| | | TAGGCGCTCACTGTTCTCTCCCTCCGCGCAGCCGAGCCACAT |
| | | CGCTGAGACAC |
| hGRP78 | 47 | AGTGCGGTTACCAGCGGAAATGCCTCGGGGTCAGAAGTCGC |
| | | AGGAGAGATAGACAGCTGCTGAACCAATGGGACCAGCGGA |
| | | TGGGGCGGATGTTATCTACCATTGGTGAACGTTAGAAACGA |
| | | ATAGCAGCCAATGAATCAGCTGGGGGGGCGGAGCAGTGAC |
| | | GTTTATTGCGGAGGGGGCCGCTTCGAATCGGCGGCGGCCAG |
| | | CTTGGTGGCCTGGGCCAATGAACGGCCTCCAACGAGCAGGG |
| | | CCTTCACCAATCGGCGGCCTCCACGACGGGGCTGGGGGAGG |
| | | GTATATAAGCCGAGTAGGCGACGGTGAGGTCGACGCCGGCC |
| | | AAGACAGCACAGACAGATTGACCTATTGGGGTGTTTCGCGA |
| | | GTGTGAGAGGGAAGCGCCGCGGCCTGTATTTCTAGACCTGC |
| | | CCTTCGCCTGGTTCGTGGCGCCTTGTGACCCCGGGCCCCTGC |
| | | CGCCTGCAAGTCGGAAATTGCGCTGTGCTCCTGTGCTACGG |
| | | CCTGTGGCTGGACTGCCTGCTGCTGCCCAACTGGCTGGCAC |
| hGRP94 | 48 | TAGTTTCATCACCACCGCCACCCCCCCGCCCCCCCGCCATCT |
| | | GAAAGGGTTCTAGGGGATTTGCAACCTCTCTCGTGTGTTTCT |
| | | TCTTTCCGAGAAGCGCCGCCACACGAGAAAGCTGGCCGCGA |
| | | AAGTCGTGCTGGAATCACTTCCAACGAAACCCCAGGCATAG |
| | | ATGGGAAAGGGTGAAGAACACGTTGCCATGGCTACCGTTTC |
| | | CCCGGTCACGGAATAAACGCTCTCTAGGATCCGGAAGTAGT |
| | | TCCGCCGCGACCTCTCTAAAAGGATGGATGTGTTCTCTGCTT |
| | | ACATTCATTGGACGTTTTCCCTTAGAGGCCAAGGCCGCCCA |
| | | GGCAAAGGGGCGGTCCCACGCGTGAGGGGCCCGCGGAGCC |
| | | ATTTGATTGGAGAAAAGCTGCAAACCCTGACCAATCGGAAG |
| | | GAGCCACGCTTCGGGCATCGGTCACCGCACCTGGACAGCTC |
| | | CGATTGGTGGACTTCCGCCCCCCCTCACGAATCCTCATTGGG |
| | | TGCCGTGGGTGCGTGGTGCGGCGCGATTGGTGGGTTCATGTT |
| | | TCCCGTCCCCCGCCCGCGAGAAGTGGGGGTGAAAAGCGGCC |
| | | CGACCTGCTTGGGGTGTAGTGGGCGGACCGCGCGGCTGGAG |
| | | GTGTGAGGATCCGAACCCAGGGGTGGGGGGTGGAGGCGGC |
| | | TCCTGCGATCGAAGGGGACTTGAGACTCACCGGCCGCACGT |
| | | C |
| hHSP70 | 49 | GGGCCGCCCACTCCCCCTTCCTCTCAGGGTCCCTGTCCCCTC |
| | | CAGTGAATCCCAGAAGACTCTGGAGAGTTCTGAGCAGGGGG |
| | | CGGCACTCTGGCCTCTGATTGGTCCAAGGAAGGCTGGGGGG |
| | | CAGGACGGGAGGCGAAAACCCTGGAATATTCCCGACCTGGC |
| | | AGCCTCATCGAGCTCGGTGATTGGCTCAGAAGGGAAAAGGC |
| | | GGGTCTCCGTGACGACTTATAAAAGCCCAGGGGCAAGCGGT |
| | | CCGGATAACGGCTAGCCTGAGGAGCTGCTGCGACAGTCCAC |
| | | TACCTTTTTCGAGAGTGACTCCCGTTGTCCCAAGGCTTCCCA |
| | | GAGCGAACCTGTGCGGCTGCAGGCACCGGCGCGTCGAGTTT |
| | | CCGGCGTCCGGAAGGACCGAGCTCTTCTCGCGGATCCAGTG |
| | | TTCCGTTTCCAGCCCCCAATCTCAGAGCGGAGCCGACAGAG |
| | | AGCAGGGAACCC |
| hKINb | 50 | GCCCCACCCCCGTCCGCGTTACAACCGGGAGGCCCGCTGGG |
| | | TCCTGCACCGTCACCCTCCTCCCTGTGACCGCCCACCTGATA |
| | | CCCAAACAACTTTCTCGCCCCTCCAGTCCCCAGCTCGCCGAG |
| | | CGCTTGCGGGGAGCCACCCAGCCTCAGTTTCCCCAGCCCCG |
| | | GGCGGGGCGAGGGGCGATGACGTCATGCCGGCGCGCGGCA |
| | | TTGTGGGGCGGGGCGAGGCGGGGCGCCGGGGGGAGCAACA |
| | | CTGAGACGCCATTTTCGGCGGCGGGAGCGGCGCAGGCGGCC |
| | | GAGCGGGACTGGCTGGGTCGGCTGGGCTGCTGGTGCGAGGA |
| | | GCCGCGGGGCTGTGCTCGGCGGCCAAGGGGACAGCGCGTG |
| | | GGTGGCCGAGGATGCTGCGGGGCGGTAGCTCCGGCGCCCCT |
| | | CGCTGGTGACTGCTGCGCCGTGCCTCACACAGCCGAGGCGG |
| | | GCTCGGCGCACAGTCGCTGCTCCGCGCTCGCGCCCGGCGGC |
| | | GCTCCAGGTGCTGACAGCGCGAGAGAGCGCGGCCTCAGGA |
| | | GCAACAC |
| hUBIb | 51 | TTCCAGAGCTTTCGAGGAAGGTTTCTTCAACTCAAATTCATC |
| | | CGCCTGATAATTTTCTTATATTTTCCTAAAGAAGGAAGAGAA |
| | | GCGCATAGAGGAGAAGGGAAATAATTTTTTAGGAGCCTTTC |
| | | TTACGGCTATGAGGAATTTGGGGCTCAGTTGAAAAGCCTAA |
| | | ACTGCCTCTCGGGAGGTTGGGCGCGGCGAACTACTTTCAGC |
| | | GGCGCACGGAGACGGCGTCTACGTGAGGGGTGATAAGTGAC |
| | | GCAACACTCGTTGCATAAATTTGCGCTCCGCCAGCCCGGAG |
| | | CATTTAGGGGCGGTTGGCTTTGTTGGGTGAGCTTGTTTGTGT |
| | | CCCTGTGGGTGGACGTGGTTGGTGATTGGCAGGATCCTGGT |
| | | ATCCGCTAACAGGTACTGGCCCACAGCCGTAAAGACCTGCG |
| | | GGGGCGTGAGAGGGGGGAATGGGTGAGGTCAAGCTGGAGG |
| | | CTTCTTGGGGTTGGGTGGGCCGCTGAGGGGAGGGGAGGGCG |
| | | AGGTGACGCGACACCCGGCCTTTCTGGGAGAGTGGGCCTTG |
| | | TTGACCTAAGGGGGGCGAGGGCAGTTGGCACGCGCACGCGC |
| | | CGACAGAAACTAACAGACATTAACCAACAGCGATTCCGTCG |
| | | CGTTTACTTGGGAGGAAGGCGGAAAAGAGGTAGTTTGTGTG |
| | | GCTTCTGGAAACCCTAAATTTGGAATCCCAGTATGAGAATG |
| | | GTGTCCCTTCTTGTGTTTCAATGGGATTTTTACTTCGCGAGTC |
| | | TTGTGGGTTTGGTTTTGTTTTCAGTTTGCCTAACACCGTGCTT |
| | | AGGTTTGAGGCAGATTGGAGTTCGGTCGGGGGAGTTTGAAT |
| | | ATCCGGAACAGTTAGTGGGGAAAGCTGTGGACGCTTGGTAA |
| | | GAGAGCGCTCTGGATTTTCCGCTGTTGACGTTGAAACCTTGA |
| | | ATGACGAATTTCGTATTAAGTGACTTAGCCTTGTAAAATTGA |
| | | GGGGAGGCTTGCGGAATATTAACGTATTTAAGGCATTTTGA |
| | | AGGAATAGTTGCTAATTTTGAAGAATATTAGGTGTAAAAGC |
| | | AAGAAATACAATGATCCTGAGGTGACACGCTTATGTTTTACT |
| | | TTTAAACTAGGTCACC |
| caffeine- | 52 | GSQVQLVESGGGLVQAGGSLRLSCTASGRTGTIYSMAWFRQA |
| binding | | PGKEREFLATVGWSSGITYYMDSVKGRFTISRDKGKNTVYLQ |
| domain (ac | | MDSLKPEDTAVYYCTATRAYSVGYDYWGQGTQVTVSS |
| V_{H}H/aCaff | | |
| V_{H}H) | | |
| cannabidiol | 53 | EVQLQASGGGFVQPGGSLRLSCAASGSTSRQYDMGWFRQAPG |
| binding | | KEREFVSAISSNQDQPPYYADSVKGRFTISRDNSKNTVYLQMN |
| domain 1 | | SLRAEDTATYYCAFKQHHANGAYWGQGTQVTVSS |
| (CA14) | | |
| cannabidiol | 54 | EVQLQASGGGFVQPGGSLRLSCAASGRFSWGEEMGWFRQAPG |
| binding | | KEREFVSAISWAATPWQYYADSVKGRFTISRDNSKNTVYLQM |
| domain 2 | | NSLRAEDTATYYCADEWHIGHVSYWGQGTQVTVSS |
| (DB6) | | |
| cannabidiol | 55 | EVQLQASGGGFVQPGGSLRLSCAASGTTSDNDTMGWFRQAPG |
| binding | | KEREFVSAISWNGGRDEYYADSVKGRFTISRDNSKNTVYLQM |
| domain 2 | | NSLRAEDTATYYCAYQDNRSWQEYWGQGTQVTVSS |
| (DB11) | | |
| cannabidiol | 56 | EVQLQASGGGFVQPGGSLRLSCAASGGYSRADDMGWFRQAP |
| binding | | GKEREFVSAISFGETDSFYYADSVKGRFTISRDNSKNTVYLQM |
| domain 2 | | NSLRAEDTATYYCAYHNYTNMFEYWGQGTQVTVSS |
| (DB18) | | |
| cannabidiol | 57 | EVQLQASGGGFVQPGGSLRLSCAASGTTYGQTNMGWFRQAPG |
| binding | | KEREFVSAISGLQGRDLYYADSVKGRFTISRDNSKNTVYLQMN |
| domain 2 | | SLRAEDTATYYCAFHDFLRMWEYWGQGTQVTVSS |
| (DB21) | | |
| NicVH | 58 | QMQLLESGPGLVKPSETLSLTCTVSGGSIWGWIRQPPGKGLEWI |
| | | GSIYSSGSTYYNPSLKSRVTTSVDTSKNQFSLRLSSVTAADTAV |
| | | YYCVAWFGDLLSLKGVELWGQGTLVTVS |
| NicVL | 59 | QSELTQPPSASGTPGQRVTISCSGSSSNIGSNYVYWYQQLPGTA |
| | | PKLLIYRNNQRPSGVPDRFSGSKSGTSASLAISGLRSEDEADYY |
| | | CAAWDDSLSAWVFGGGTQLDILG |
| FKBP | 60 | GVQVETISPGDGRTFPKRGQTCVVHYTGMLEDGKKFDSSRDR |
| | | NKPFKFMLGKQEVIRGWEEGVAQMSVGQRAKLTISPDYAYGA |
| | | TGHPGIIPPHATLVFDVELLKLE |
| FRB | 61 | ILWHEMWHEGLEEASRLYFGERNVKGMFEVLEPLHAMMERG |
| | | PQTLKETSFNQAYGRDLMEAQEWCRKYMKSGNVKDLTQAW |
| | | DLYYHVFRRISK |
| Exemplary | 62 | GGCGTAGCCGATGTCGCG |
| Zinc Finger | | |
| Binding Site | | |
| 4X Zinc | 75 | cgggtttcgtaacaatcgcatgaggattcgcaacgccttcGGCGTAGCCGATGTCGC |
| Finger | | GctcccgtctcagtaaaggtcGGCGTAGCCGATGTCGCGcaatcggactgccttcg |
| Binding Site | | tacGGCGTAGCCGATGTCGCG |
| Exemplary | 64 | TCTAGAGGGTATATAATGGGGGCCA |
| Core | | |
| Promoter | | |
| Sequence | | |
| Exemplary | 65 | cgggtttcgtaacaatcgcatgaggattcgcaacgccttcGGCGTAGCCGATGTCGC |
| ITF- | | GctcccgtctcagtaaaggtcGGCGTAGCCGATGTCGCGcaatcggactgccttcg |
| Responsive | | tacGGCGTAGCCGATGTCGCGcgtatcagtcgcctcggaacGGCGTAGCC |
| Promoter | | GATGTCGCGcattcgtaagaggctcactctcccttacacggagtggataACTAGTTC |
| | | TAGAGGGTATATAATGGGGGCCA |
| WPRE | 66 | TCTGTTCCTGTTAATCAACCTCTGGATTACAAAATTTGTGAA |
| | | AGATTGACTGATATTCTTAACTATGTTGCTCCTTTTACGCTGT |
| | | GTGGATATGCTGCTTTAATGCCTCTGTATCATGCTATTGCTT |
| | | CCCGTACGGCTTTCGTTTTCTCCTCCTTGTATAAATCCTGGTT |
| | | GCTGTCTCTTTATGAGGAGTTGTGGCCCGTTGTCCGTCAACG |
| | | TGGCGTGGTGTGCTCTGTGTTTGCTGACGCAACCCCCACTGG |
| | | CTGGGGCATTGCCACCACCTGTCAACTCCTTTCTGGGACTTT |
| | | CGCTTTCCCCCTCCCGATCGCCACGGCAGAACTCATCGCCGC |
| | | CTGCCTTGCCCGCTGCTGGACAGGGGCTAGGTTGCTGGGCA |
| | | CTGATAATTCCGTGGTGTTGTCGGGGAAGCTGACGTCCTTTC |
| | | CATGGCTGCTCGCCTGTGTTGCCAACTGGATCCTGCGCGGG |
| | | ACGTCCTTCTGCTACGTCCCTTCGGCTCTCAATCCAGCGGAC |
| | | CTCCCTTCCCGAGGCCTTCTGCCGGTTCTGCGGCCTCTCCCG |
| | | CGTCTTCGCTTTCGGCCTCCGACGAGTCGGATCTCCCTTTGG |
| | | GCCGCCTCCCCGCCTGTTTCGCCTCGGCGTCCGGTCCGTGTT |
| | | GCTTGGTCGTCACCTGTGCAGAATTGCGAACCATGGATTCCA |
| WPRE | 67 | TCTGTTCCTGTTAATCAACCTCTGGATTACAAAATTTGTGAA |
| | | AGATTGACTGATATTCTTAACTATGTTGCTCCTTTTACGCTGT |
| | | GTGGATATGCTGCTTTAATGCCTCTGTATCATGCTATTGCTT |
| | | CCCGTACGGCTTTCGTTTTCTCCTCCTTGTATAAATCCTGGTT |
| | | GCTGTCTCTTTATGAGGAGTTGTGGCCCGTTGTCCGTCAACG |
| | | TGGCGTGGTGTGCTCTGTGTTTGCTGACGCAACCCCCACTGG |
| | | CTGGGGCATTGCCACCACCTGTCAACTCCTTTCTGGGACTTT |
| | | CGCTTTCCCCCTCCCGATCGCCACGGCAGAACTCATCGCCGC |
| | | CTGCCTTGCCCGCTGCTGGACAGGGGCTAGGTTGCTGGGCA |
| | | CTGATAATTCCGTGGTGTTGTCGGGGAAATCATCGTCCTTTC |
| | | CTTGGCTGCTCGCCTGTGTTGCCAACTGGATCCTGCGCGGGA |
| | | CGTCCTTCTGCTACGTCCCTTCGGCTCTCAATCCAGCGGACC |
| | | TCCCTTCCCGAGGCCTTCTGCCGGTTCTGCGGCCTCTCCCGC |
| | | GTCTTCGCTTTCGGCCTCCGACGAGTCGGATCTCCCTTTGGG |
| | | CCGCCTCCCCGCCTGTTTCGCCTCGGCGTCCGGTCCGTGTTG |
| | | CTTGGTCGTCACCTGTGCAGAATTGCGAACCATGGATTCCA |
| Progesterone | 68 | GIRKFKKFNKVRVVRALDAVALPQPLGVPNESQALSQRFTFSP |
| Receptor | | GQDIQLIPPLINLLMSIEPDVIYAGHDNTKPDTSSSLLTSLNQLGE |
| Domain | | RQLLSVVKWSKSLPGFRNLHIDDQITLIQYSWMSLMVFGLGW |
| | | RSYKHVSGQMLYFAPDLILNEQRMKESSFYSLCLTMWQIPQEF |
| | | VKLQVSQEEFLCMKVLLLLNTIPLEGLRSQTQFEEMRSSYIRELI |
| | | KAIGLRQKGVVSSSQRFYQLTKLLDNLHDLVKQLHLYCLNTFI |
| | | QSRALSVEFPEMMSEVIAAQLPKILAGMVKPLLFHKK |
| p65 VPR | 69 | CAGTACCTGCCAGATACAGACGATCGTCACCGGATTGAGGA |
| domain | | GAAACGTAAAAGGACATATGAGACCTTCAAGAGCATCATGA |
| | | AGAAGAGTCCTTTCAGCGGACCCACCGACCCCCGGCCTCCA |
| | | CCTCGACGCATTGCTGTGCCTTCCCGCAGCTCAGCTTCTGTC |
| | | CCCAAGCCAGCACCCCAGCCCTATCCCTTTACGTCATCCCTG |
| | | AGCACCATCAACTATGATGAGTTTCCCACCATGGTGTTTCCT |
| | | TCTGGGCAGATCAGCCAGGCCTCGGCCTTGGCCCCGGCCCC |
| | | TCCCCAAGTCCTGCCCCAGGCTCCAGCCCCTGCCCCTGCTCC |
| | | AGCCATGGTATCAGCTCTGGCCCAGGCCCCAGCCCCTGTCC |
| | | CAGTCCTAGCCCCAGGCCCTCCTCAGGCTGTGGCCCCACCTG |
| | | CCCCCAAGCCCACCCAGGCTGGGGAAGGAACGCTGTCAGAG |
| | | GCCCTGCTGCAGCTGCAGTTTGATGATGAAGACCTGGGGGC |
| | | CTTGCTTGGCAACAGCACAGACCCAGCTGTGTTCACAGACC |
| | | TGGCATCCGTCGACAACTCCGAGTTTCAGCAGCTGCTGAAC |
| | | CAGGGCATACCTGTGGCCCCCCACACAACTGAGCCCATGCT |
| | | GATGGAGTACCCTGAGGCTATAACTCGCCTAGTGACAGGGG |
| | | CCCAGAGGCCCCCCGACCCAGCTCCTGCTCCACTGGGGGCC |
| | | CCGGGGCTCCCCAATGGCCTCCTTTCAGGAGATGAAGACTT |
| | | CTCCTCCATTGCGGACATGGACTTCTCAGCCCTGCTG |
| p65 VPR | 70 | QYLPDTDDRHRIEEKRKRTYETFKSIMKKSPFSGPTDPRPPPRRI |
| domain | | AVPSRSSASVPKPAPQPYPFTSSLSTINYDEFPTMVFPSGQISQA |
| | | SALAPAPPQVLPQAPAPAPAPAMVSALAQAPAPVPVLAPGPPQ |
| | | AVAPPAPKPTQAGEGTLSEALLQLQFDDEDLGALLGNSTDPAV |
| | | FTDLASVDNSEFQQLLNQGIPVAPHTTEPMLMEYPEAITRLVTG |
| | | AQRPPDPAPAPLGAPGLPNGLLSGDEDFSSIADMDFSALL |
| E2A-T2A | 71 | CAGTGTACCAACTACGCCCTGCTGAAACTGGCCGGCGACGT |
| (linker | | GGAATCTAATCCTGGACCTGGATCTGGCGAGGGACGCGGGA |
| italicized) | | GTCTACTGACGTGTGGAGACGTGGAGGAAAACCCTGGACCT |
| E2A-T2A | 72 | QCTNYALLKLAGDVESNPGPGSGEGRGSLLTCGDVEENPGP |
| (linker | | |
| italicized) | | |
| BFP | 73 | atgAGCGAGCTGATTAAGGAGAACATGCACATGAAGCTGTAC |
| | | ATGGAGGGCACCGTGGACAACCATCACTTCAAGTGCACATC |
| | | CGAGGGCGAAGGCAAGCCCTACGAGGGCACCCAGACCATG |
| | | AGAATCAAGGTGGTCGAGGGCGGCCCTCTCCCCTTCGCCTT |
| | | CGACATCCTGGCTACTAGCTTCCTCTACGGCAGCAAGACCTT |
| | | CATCAACCACACCCAGGGCATCCCCGACTTCTTCAAGCAGT |
| | | CCTTCCCTGAGGGCTTCACATGGGAGAGAGTCACCACATAC |
| | | GAAGACGGGGGCGTGCTGACCGCTACCCAGGACACCAGCCT |
| | | CCAGGACGGCTGCCTCATCTACAACGTCAAGATCAGAGGGG |
| | | TGAACTTCACATCCAACGGCCCTGTGATGCAGAAGAAAACA |
| | | CTCGGCTGGGAGGCCTTCACCGAGACGCTGTACCCCGCTGA |
| | | CGGCGGCCTGGAAGGCAGAAACGACATGGCCCTGAAGCTC |
| | | GTGGGCGGGAGCCATCTGATCGCAAACATCAAGACCACATA |
| | | TAGATCCAAGAAACCCGCTAAGAACCTCAAGATGCCTGGCG |
| | | TCTACTATGTGGACTACAGACTGGAAAGAATCAAGGAGGCC |
| | | AACAACGAGACCTACGTCGAGCAGCACGAGGTGGCAGTGG |
| | | CCAGATACTGCGACCTCCCTAGCAAACTGGGGCACAAGCTT |
| | | AAT |
| BFP | 74 | MSELIKENMHMKLYMEGTVDNHHFKCTSEGEGKPYEGTQTM |
| | | RIKVVEGGPLPFAFDILATSFLYGSKTFINHTQGIPDFFKQSFPEG |
| | | FTWERVTTYEDGGVLTATQDTSLQDGCLIYNVKIRGVNFTSNG |
| | | PVMQKKTLGWEAFTETLYPADGGLEGRNDMALKLVGGSHLIA |
| | | NIKTTYRSKKPAKNLKMPGVYYVDYRLERIKEANNETYVEQH |
| | | EVAVARYCDLPSKLGHKLN |
| f | | |

While the present disclosure has been particularly shown and described with reference to a preferred embodiment and various alternate embodiments, it will be understood by persons skilled in the relevant art that various changes in form and details can be made therein without departing from the spirit and scope of the present disclosure and appended claims.

### INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### EQUIVALENTS

While various specific embodiments have been illustrated and described, the above specification is not restrictive. It will be appreciated that various changes can be made without departing from the spirit and scope of the present disclosure(s). Many variations will become apparent to those skilled in the art upon review of this specification.

1.-15. (canceled)

16. An engineered cell comprising an inducible transcription factor (ITF), wherein the ITF comprises:
a first monomer, wherein the first monomer comprises a DNA binding domain and a first ligand binding domain; and a second monomer, wherein the second monomer comprises a transcriptional effector domain and a second ligand binding domain,
wherein each monomer is oligomerizable via a cognate ligand that binds to each ligand binding domain,
wherein ligand-induced oligomerization forms the ITF,
wherein the cognate ligand is selected from the group consisting of: caffeine, a cannabidiol (CBD), a phytocannabinoid, nicotine, rapamycin, AP1903, AP20187, FK1012, derivatives thereof, and analogs thereof,
optionally wherein the DNA binding domain comprises a zinc finger (ZF) protein domain,
optionally wherein each ligand binding domain comprises a single-domain antibody, wherein the single-domain antibody comprises a V_{H}H, and
optionally wherein the ITF is capable of modulating transcription of a gene of interest operably linked to an ITF-responsive promoter.

17. The engineered cell of claim 16, wherein:
a. the V_{H}H is an anti-caffeine V_{H}H (ac V_{H}H), optionally wherein the anti-caffeine V_{H}H (ac V_{H}H) comprises the amino acid sequence of SEQ ID NO: 52, and/or optionally wherein each of the first ligand binding domain and the second ligand binding domain comprises the ac V_{H}H; or
b. the cognate ligand is a cannabidiol or a phytocannabinoid, and the V_{H}H comprises an amino acid sequence selected from the group consisting of SEQ ID NO: 53, SEQ ID NO: 54, SEQ ID NO: 55, SEQ ID NO: 56, and SEQ ID NO: 57 (CA-14, DB-6, DB-11, DB-18, and DB-21, respectively), optionally wherein one of the first or the second ligand binding domain comprises a V_{H}H comprising the amino acid sequence of SEQ ID NO: 53 (CA-14) and the other of the first or the second binding domain comprises a V_{H}H comprising an amino acid sequence selected from the group consisting of: SEQ ID NO: 54, SEQ ID NO: 55, SEQ ID NO: 56, and SEQ ID NO: 57 (DB-6, DB-11, DB-18, and DB-21, respectively), optionally wherein one of the first or the second ligand binding domain comprises a V_{H}H comprising the amino acid sequence of SEQ ID NO: 53 (CA-14) and the other of the first or the second binding domain comprises a V_{H}H comprising an amino acid sequence selected from the group consisting of: SEQ ID NO: 54, SEQ ID NO: 55, SEQ ID NO: 56, and SEQ ID NO: 57 (DB-6, DB-11, DB-18, and DB-21, respectively), optionally wherein one of the first or the second ligand binding domain comprises a V_{H}H comprising the amino acid sequence of SEQ ID NO: 53 (CA-14) and the other of the first or the second binding domain comprises a V_{H}H comprising the amino acid sequence of SEQ ID NO: 57 (DB-21); or
c. each ligand binding domain comprises an anti-nicotine antibody heavy chain variable domain (anti-Nic VH) or an anti-nicotine antibody light chain variable domain (anti-Nic VL), optionally wherein the anti-Nic VH comprises the amino acid sequence of SEQ ID NO: 58 and the anti-Nic VL comprises the amino acid sequence of SEQ ID NO: 59, optionally wherein one of the first or the second ligand binding domain comprises the anti-Nic VH and the other of the first or the second ligand binding domain comprises the anti-Nic VL; or
d. one of the first or the second ligand binding domain comprises an FKBP domain, and the other of the first or the second ligand binding domain comprises an FRB domain, optionally wherein the FKBP comprises the amino acid sequence of SEQ ID NO: 60, optionally wherein the FRB domain comprises the amino acid sequence of SEQ ID NO: 61; or
e. the first ligand binding domain comprises the FKBP domain and the second ligand binding domain comprises the FRB domain, optionally wherein the FKBP comprises the amino acid sequence of SEQ ID NO: 60, optionally wherein the FRB domain comprises the amino acid sequence of SEQ ID NO: 61; or
f. one of the first or the second ligand binding domain comprises the FRB domain, and the other of the first or the second ligand binding domain comprises the FKBP domain, optionally wherein the FKBP comprises the amino acid sequence of SEQ ID NO: 60, optionally wherein the FRB domain comprises the amino acid sequence of SEQ ID NO: 61; or
g. the first ligand binding domain comprises the FRB domain and the second ligand binding domain comprises the FKBP domain, optionally wherein the FKBP comprises the amino acid sequence of SEQ ID NO: 60, optionally wherein the FRB domain comprises the amino acid sequence of SEQ ID NO: 61.

18. The engineered cell of claim 16, wherein:
a. the first monomer and/or the second monomer further comprises a nuclear localization signal (NLS),
optionally wherein the NLS comprises an amino acid sequence selected from the group consisting of: SEQ ID NO: 2, SEQ ID NO: 3, and SEQ ID NO: 4; and/or
b. the second monomer further comprises a nuclear export signal (NES), optionally wherein the NES comprises an amino acid sequence selected from the group consisting of: SEQ ID NO: 5, SEQ ID NO: 6, SEQ ID NO: 7 SEQ ID NO: 8, SEQ ID NO: 9, SEQ ID NO: 10, SEQ ID NO: 11, SEQ ID NO: 12, SEQ ID NO: 13, and SEQ ID NO: 14.

19. The engineered cell of claim 16, wherein:
a. the ZF protein domain is modular in design and is composed of one or more zinc finger arrays (ZFA);
b. the ZF protein domain comprises an array of one to ten zinc finger motifs; and/or
c. the ZF protein domain comprises an array of six zinc finger motifs.

20. The engineered cell of claim 16, wherein:
a. the transcriptional effector domain comprises a transcriptional activation domain, optionally wherein the transcriptional activation domain is selected from the group consisting of: a Herpes Simplex Virus Protein 16 (VP16) activation domain; an activation domain comprising four tandem copies of VP16; a VP64 activation domain; a p65 activation domain of NFκB; an Epstein-Barr virus R transactivator (Rta) activation domain; a tripartite activator comprising the VP64, the p65, and the Rta activation domains (VPR activation domain); and a histone acetyltransferase (HAT) core domain of the human E1A-associated protein p300 (p300 HAT core activation domain); or
b. the transcriptional effector domain comprises a transcriptional repressor domain, optionally wherein the transcriptional repressor domain is selected from the group consisting of: a Krüppel associated box (KRAB) repression domain; a truncated Krüppel associated box (KRAB) repression domain; a Histone Deacetylase 4 (HDAC4) repressor domain; a Scleraxis (SCX) HLH domain, an Inhibitor of DNA binding 1 (ID1) HLH domain, a HECT domain and RCC1-like domain-containing protein 2 (HERC2) Cyt-b5 domain, a Twist-related protein 1 (TWST1) HLH domain, an Homeobox protein Nkx-2.2 (NKX22) homeodomain, an Inhibitor of DNA binding 1 (ID3) HLH domain, and a Twist-related protein 2 (TWST2) HLH domain, and EED repressor domain; a Repressor Element Silencing Transcription Factor (REST) repression domain; a WRPW motif (SEQ ID NO: 76) of the hairy-related basic helix-loop-helix repressor proteins, the motif is known as a WRPW (SEQ ID NO: 76) repression domain; a DNA (cytosine-5)-methyltransferase 3B (DNMT3B) repression domain; and an HP1 alpha chromoshadow repression domain.

21. The engineered cell of claim 16, wherein:
a. the cell comprises a human cell; and/or
b. the cell comprises a stem cell; and/or
c. the cell comprises an immune cell; and/or
d. the cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an erythrocyte, a platelet cell, a human embryonic stem cell (ESC), an ESC-derived cell, a pluripotent stem cell, a mesenchymal stromal cell (MSC), an induced pluripotent stem cell (iPSC), and an iPSC-derived cell.

22. A genetic switch for modulating transcription of a gene of interest, comprising the engineered cell of claim 16 and a cognate ligand selected from the group consisting of: caffeine, a cannabidiol (CBD), a phytocannabinoid, nicotine, rapamycin, AP1903, AP20187, FK1012, derivatives thereof, and analogs thereof, to induce formation of the inducible transcription factor (ITF).

23. A method of modulating transcription of a gene of interest, comprising:
a. contacting the engineered cell of claim 16 with a cognate ligand selected from the group consisting of: caffeine, a cannabidiol (CBD), a phytocannabinoid, and nicotine, to induce formation of the inducible transcription factor (ITF), optionally further comprising culturing the engineered cell for a time period sufficient to allow the ITF to modulate transcription of a gene operably linked to an ITF-responsive promoter, optionally wherein the contacting is performed in a human or animal, and optionally wherein contacting the transformed cell with the cognate ligand comprises administering a pharmacological dose of the cognate ligand to the human or animal; or
b. contacting the engineered cell of claim 16 with a cognate ligand selected from the group consisting of: rapamycin, AP1903, AP20187, FK1012, derivatives thereof, and analogs thereof to induce formation of the inducible transcription factor (ITF), optionally further comprising culturing the engineered cell for a time period sufficient to allow the ITF to modulate transcription of a gene operably linked to an ITF-responsive promoter, optionally wherein the engineered cell is in a human or animal, and wherein contacting the engineered cell with the cognate ligand comprises administering a pharmacological dose of the rapamycin to the human or animal, optionally wherein the transcription effector domain of the second monomer comprises a transcriptional activation domain and modulating transcription comprises activating transcription of the gene of interest, or wherein the transcription effector domain of the second monomer comprises a transcriptional repressor domain and modulating transcription comprises repressing transcription of the gene of interest.

24. An engineered cell comprising an inducible transcription factor (ITF), wherein the ITF comprises a DNA binding domain, a transcriptional effector domain, and a ligand binding domain,
wherein the ITF undergoes nuclear localization upon binding of the ligand binding domain to a cognate ligand,
wherein when localized to a cell nucleus, the ITF is capable of modulating transcription of a gene of interest operably linked to an ITF-responsive promoter,
wherein the cognate ligand is mifepristone or a derivative thereof, optionally wherein the ligand binding domain comprises a progesterone receptor domain and the progesterone receptor domain comprises the amino acid sequence of SEQ ID NO: 68,
optionally wherein the DNA binding domain comprises a zinc finger (ZF) protein domain,
optionally wherein the ITF further comprises a peptide linker between the DNA binding domain and the transcriptional effector domain,
optionally wherein the DNA binding domain binds to the ITF-responsive promoter, and
optionally wherein the cell further comprises an expression cassette comprising the ITF-responsive promoter operably linked to an exogenous polynucleotide sequence encoding the gene of interest,
optionally wherein the ITF-responsive promoter comprises an ITF-binding domain sequence and a core promoter sequence,
optionally wherein the core promoter sequence comprises a minimal promoter and the minimal promoter is selected from the group consisting of: minP, minCMV, YB_TATA, minTATA, and minTK, or wherein the core promoter sequence is derived from a constitutive promoter and the core promoter sequence is derived from a constitutive promoter selected from the group consisting of: CMV, EFS, SFFV, SV40, MND, PGK, UbC, EF1a, hCAGG, hACTb, heIF4A1, hGAPDH, hGRP78, hGRP94, hHSP70, hKINb, and hUBIb.

25. The engineered cell of claim 24, wherein:
a. the ZF protein domain is modular in design and is composed of one or more zinc finger arrays (ZFA);
b. the ZF protein domain comprises an array of one to ten zinc finger motifs; and/or
c. the ZF protein domain comprises an array of six zinc finger motifs.

26. The engineered cell of claim 24, wherein:
a. the transcriptional effector domain comprises a transcriptional activation domain, optionally wherein the transcriptional activation domain is selected from the group consisting of: a Herpes Simplex Virus Protein 16 (VP16) activation domain; an activation domain comprising four tandem copies of VP16; a VP64 activation domain; a p65 activation domain of NFκB; an Epstein-Barr virus R transactivator (Rta) activation domain; a tripartite activator comprising the VP64, the p65, and the Rta activation domains (VPR activation domain); and a histone acetyltransferase (HAT) core domain of the human E1A-associated protein p300 (p300 HAT core activation domain); or
b. the transcriptional effector domain comprises a transcriptional repressor domain, optionally wherein the transcriptional repressor domain is selected from the group consisting of: a Krüppel associated box (KRAB) repression domain; a truncated Krüppel associated box (KRAB) repression domain; a Histone Deacetylase 4 (HDAC4) repressor domain; a Scleraxis (SCX) HLH domain, an Inhibitor of DNA binding 1 (ID1) HLH domain, a HECT domain and RCC1-like domain-containing protein 2 (HERC2) Cyt-b5 domain, a Twist-related protein 1 (TWST1) HLH domain, an Homeobox protein Nkx-2.2 (NKX22) homeodomain, an Inhibitor of DNA binding 1 (ID3) HLH domain, and a Twist-related protein 2 (TWST2) HLH domain, and EED repressor domain; a Repressor Element Silencing Transcription Factor (REST) repression domain; a WRPW motif (SEQ ID NO: 76) of the hairy-related basic helix-loop-helix repressor proteins, the motif is known as a WRPW (SEQ ID NO: 76) repression domain; a DNA (cytosine-5)-methyltransferase 3B (DNMT3B) repression domain; and an HP1 alpha chromoshadow repression domain.

27. The engineered cell of claim 24, wherein:
a. the cell comprises a human cell; and/or
b. the cell comprises a stem cell; and/or
c. the cell comprises an immune cell; and/or
d. the cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an erythrocyte, a platelet cell, a human embryonic stem cell (ESC), an ESC-derived cell, a pluripotent stem cell, a mesenchymal stromal cell (MSC), an induced pluripotent stem cell (iPSC), and an iPSC-derived cell.

28. A genetic switch for modulating transcription of a gene of interest, comprising the engineered cell of claim 24 and mifepristone, or a derivative thereof.

29. A method of modulating transcription of a gene of interest, comprising: contacting the engineered cell of claim 24 with mifepristone, or a derivative thereof, to induce nuclear localization of the inducible transcription factor (ITF), optionally further comprising culturing the engineered cell for a time period sufficient to allow the ITF to modulate transcription of a gene operably linked to an ITF-responsive promoter, optionally wherein the engineered cell is in a human or animal, and wherein contacting the engineered cell with the mifepristone comprises administering a pharmacological dose of the mifepristone to the human or animal, optionally wherein the transcriptional effector domain comprises a transcriptional activation domain and modulating transcription comprises activating transcription of the gene of interest, or wherein the transcriptional effector domain comprises a transcriptional repressor domain and modulating transcription comprises repressing transcription of the gene of interest.

30. An expression system comprising:
a) a first expression cassette comprising a first promoter and an exogenous polynucleotide sequence encoding a first monomer, wherein the first monomer comprises a DNA binding domain and a first ligand binding domain; and a second expression cassette comprising a second promoter and an exogenous polynucleotide sequence encoding a second monomer, wherein the second monomer comprises a transcription effector domain and a second ligand binding domain, wherein each monomer is oligomerizable via a cognate ligand that binds to each ligand binding domain, wherein ligand-induced oligomerization of the first and the second monomer forms an inducible transcription factor (ITF), and wherein the cognate ligand is: selected from the group consisting of: caffeine, a cannabidiol (CBD), a phytocannabinoid, nicotine, rapamycin, AP1903, AP20187, FK1012, derivatives thereof, and analogs thereof; or
b) a first promoter and an exogenous polynucleotide sequence encoding an inducible transcription factor (ITF), wherein the ITF comprises a DNA binding domain, a transcriptional effector domain, and a ligand binding domain, wherein the ITF undergoes nuclear localization upon binding of the ligand binding domain to a cognate ligand, wherein when localized to a cell nucleus, the ITF is capable of modulating transcription of a gene of interest operably linked to an ITF-responsive promoter, wherein the cognate ligand is mifepristone or a derivative thereof,
optionally wherein the DNA binding domain comprises a zinc finger (ZF) protein domain,
optionally wherein the ZF protein domain is modular in design and is composed of one or more zinc finger arrays (ZFA),
optionally wherein the ZF protein domain comprises an array of one to ten zinc finger motifs, and
optionally wherein the expression system further comprises a third expression cassette comprising an ITF-responsive promoter operably linked to a gene of interest, wherein the ITF-responsive promoter comprises a core promoter sequence and a sequence that binds to the DNA binding domain of the first engineered polypeptide.

31. The engineered cell of claim 30, wherein:
a. the ZF protein domain is modular in design and is composed of one or more zinc finger arrays (ZFA); and/or
b. the ZF protein domain comprises an array of one to ten zinc finger motifs.

32. An engineered cell comprising the expression system of claim 30.

33. The engineered cell of claim 32, wherein:
a. the cell comprises a human cell; and/or
b. the cell comprises a stem cell; and/or
c. the cell comprises an immune cell; and/or
d. the cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an erythrocyte, a platelet cell, a human embryonic stem cell (ESC), an ESC-derived cell, a pluripotent stem cell, a mesenchymal stromal cell (MSC), an induced pluripotent stem cell (iPSC), and an iPSC-derived cell.


SEQUENCE LISTING (76 sequences)

SEQ ID NO: 1 - protein, 176 aa, synthetic construct
  REGION 1..176: note = Description of Artificial Sequence: Synthetic polypeptide
SRPGERPFQCRICMRNFSRRHGLDRHTRTHTGEKPFQCRICMRNFSDHSSLKRHLRTHTGSQKPFQCRICMRNFSVRHNLTRHLRTHTGEKPFQCRICMRNFSDHSNLSRHLKTHTGSQKPFQCRICMRNFSQRSSLVRHLRTHTGEKPFQCRICMRNFSESGHLKRHLRTHLRGS

SEQ ID NO: 2 - protein, 8 aa, synthetic construct
  REGION 1..8: note = Description of Artificial Sequence: Synthetic peptide
PKKKRKVG

SEQ ID NO: 3 - protein, 16 aa, synthetic construct
  REGION 1..16: note = Description of Artificial Sequence: Synthetic peptide
KRPAATKKRGQRKKKK

SEQ ID NO: 4 - protein, 10 aa, synthetic construct
  REGION 1..10: note = Description of Artificial Sequence: Synthetic peptide
AAFEDLRVLS

SEQ ID NO: 5 - protein, 18 aa, synthetic construct
  REGION 1..18: note = Description of Artificial Sequence: Synthetic peptide
DNGMEELSQALASSFSVS

SEQ ID NO: 6 - protein, 14 aa, synthetic construct
  REGION 1..14: note = Description of Artificial Sequence: Synthetic peptide
MEELSQALASSFSV

SEQ ID NO: 7 - protein, 12 aa, synthetic construct
  REGION 1..12: note = Description of Artificial Sequence: Synthetic peptide
PLQLPPLERLTL

SEQ ID NO: 8 - protein, 12 aa, synthetic construct
  REGION 1..12: note = Description of Artificial Sequence: Synthetic peptide
NELALKLAGLDI

SEQ ID NO: 9 - protein, 15 aa, synthetic construct
  REGION 1..15: note = Description of Artificial Sequence: Synthetic peptide
ERFEMFRELNEALEL

SEQ ID NO: 10 - protein, 15 aa, synthetic construct
  REGION 1..15: note = Description of Artificial Sequence: Synthetic peptide
DHAEKVAEKLEALSV

SEQ ID NO: 11 - protein, 15 aa, synthetic construct
  REGION 1..15: note = Description of Artificial Sequence: Synthetic peptide
QLVEELLKIICAFQL

SEQ ID NO: 12 - protein, 15 aa, synthetic construct
  REGION 1..15: note = Description of Artificial Sequence: Synthetic peptide
TNLEALQKKLEELEL

SEQ ID NO: 13 - protein, 15 aa, synthetic construct
  REGION 1..15: note = Description of Artificial Sequence: Synthetic peptide
DVKEEMTSALATMRV

SEQ ID NO: 14 - protein, 15 aa, synthetic construct
  REGION 1..15: note = Description of Artificial Sequence: Synthetic peptide
STNGSLAAEFRHLQL

SEQ ID NO: 15 - protein, 8 aa, synthetic construct
  REGION 1..8: note = Description of Artificial Sequence: Synthetic peptide
GSGSGSGS

SEQ ID NO: 16 - protein, 4 aa, synthetic construct
  REGION 1..4: note = Description of Artificial Sequence: Synthetic peptide
KEGS

SEQ ID NO: 17 - protein, 5 aa, synthetic construct
  REGION 1..5: note = Description of Artificial Sequence: Synthetic peptide
EAAAK

SEQ ID NO: 18 - protein, 7 aa, synthetic construct
  REGION 1..7: note = Description of Artificial Sequence: Synthetic peptide
AAPAKQE

SEQ ID NO: 19 - protein, 27 aa, synthetic construct
  REGION 1..27: note = Description of Artificial Sequence: Synthetic peptide
GSGSGSGSGGAEAAAKEAAAKEAAAKA

SEQ ID NO: 20 - protein, 44 aa, synthetic construct
  REGION 1..44: note = Description of Artificial Sequence: Synthetic polypeptide
AAPAKQEAAAPAKQEAAAPAKQEAAAPAPAAKAEAPAAAPAAKA

SEQ ID NO: 21 - protein, 17 aa, synthetic construct
  REGION 1..17: note = Description of Artificial Sequence: Synthetic peptide
AEAAAKEAAAKEAAAKA

SEQ ID NO: 22 - DNA, 31 bp, synthetic construct
  misc_feature 1..31: note = Description of Artificial Sequence: Synthetic oligonucleotide
  source 1..31: mol_type = other DNA
agagggtatataatggaagctcgacttccag

SEQ ID NO: 23 - DNA, 52 bp, synthetic construct
  misc_feature 1..52: note = Description of Artificial Sequence: Synthetic oligonucleotide
  source 1..52: mol_type = other DNA
gggaatttccggggactttccgggaatttccggggactttccgggaatttcc

SEQ ID NO: 24 - DNA, 85 bp, synthetic construct
  misc_feature 1..85: note = Description of Artificial Sequence: Synthetic oligonucleotide
  source 1..85: mol_type = other DNA
caccagacagtgacgtcagctgccagatcccatggccgtcatactgtgacgtctttcagacaccccattgacgtcaatgggagaa

SEQ ID NO: 25 - DNA, 90 bp, synthetic construct
  misc_feature 1..90: note = Description of Artificial Sequence: Synthetic oligonucleotide
  source 1..90: mol_type = other DNA
ggaggaaaaactgtttcatacagaaggcgtggaggaaaaactgtttcatacagaaggcgtggaggaaaaactgtttcatacagaaggcgt

SEQ ID NO: 26 - DNA, 115 bp, synthetic construct
  misc_feature 1..115: note = Description of Artificial Sequence: Synthetic polynucleotide
  source 1..115: mol_type = other DNA
aggatgtccatattaggacatctaggatgtccatattaggacatctaggatgtccatattaggacatctaggatgtccatattaggacatctaggatgtccatattaggacatct

SEQ ID NO: 27 - DNA, 115 bp, synthetic construct
  misc_feature 1..115: note = Description of Artificial Sequence: Synthetic polynucleotide
  source 1..115: mol_type = other DNA
agtatgtccatattaggacatctaccatgtccatattaggacatctactatgtccatattaggacatcttgtatgtccatattaggacatctaaaatgtccatattaggacatct

SEQ ID NO: 28 - DNA, 48 bp, synthetic construct
  misc_feature 1..48: note = Description of Artificial Sequence: Synthetic oligonucleotide
  source 1..48: mol_type = other DNA
tgagtcagtgactcagtgagtcagtgactcagtgagtcagtgactcag

SEQ ID NO: 29 - DNA, 120 bp, synthetic construct
  misc_feature 1..120: note = Description of Artificial Sequence: Synthetic polynucleotide
  source 1..120: mol_type = other DNA
agatcaaagggtttaagatcaaagggcttaagatcaaagggtataagatcaaagggcctaagatcaaagggactaagatcaaagggtttaagatcaaagggcttaagatcaaagggccta

SEQ ID NO: 30 - DNA, 32 bp, synthetic construct
  misc_feature 1..32: note = Description of Artificial Sequence: Synthetic oligonucleotide
  source 1..32: mol_type = other DNA
gtctagacgtctagacgtctagacgtctagac

SEQ ID NO: 31 - DNA, 24 bp, synthetic construct
  misc_feature 1..24: note = Description of Artificial Sequence: Synthetic oligonucleotide
  source 1..24: mol_type = other DNA
cagacacagacacagacacagaca

SEQ ID NO: 32 - DNA, 65 bp, synthetic construct
  misc_feature 1..65: note = Description of Artificial Sequence: Synthetic oligonucleotide
  source 1..65: mol_type = other DNA
ggatccggtactcgagatctgcgatctaagtaagcttggcattccggtactgttggtaaagccac

SEQ ID NO: 33 - DNA, 588 bp, unidentified
  misc_feature 1..588: note = Description of Artificial Sequence: Synthetic polynucleotide
  source 1..588: mol_type = other DNA; note = Cytomegalovirus
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctc

SEQ ID NO: 34 - DNA, 1179 bp, synthetic construct
  misc_feature 1..1179: note = Description of Artificial Sequence: Synthetic polynucleotide
  source 1..1179: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgccgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcgaccaccgagaatcggacgggggtagtctcaagctggccggcctgctctggtgcctgtcctcgcgccgccgtgtatcgccccgccccgggcggcaaggctggcccggtcggcaccagttgcgtgagcggaaagatggccgcttcccggtcctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 35 - DNA, 544 bp, synthetic construct
  misc_feature 1..544: note = Description of Artificial Sequence: Synthetic polynucleotide
  source 1..544: mol_type = other DNA
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 36 - DNA, 399 bp, synthetic construct
  misc_feature 1..399: note = Description of Artificial Sequence: Synthetic polynucleotide
  source 1..399: mol_type = other DNA
tttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagccca

SEQ ID NO: 37 - DNA, 511 bp, synthetic construct
  misc_feature 1..511: note = Description of Artificial Sequence: Synthetic polynucleotide
  source 1..511: mol_type = other DNA
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 38 - DNA, 408 bp, unidentified
  misc_feature 1..408: note = Description of Artificial Sequence: Synthetic polynucleotide
  source 1..408: mol_type = other DNA; note = Spleen focus-forming virus
gtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccggg

SEQ ID NO: 39 - DNA, 344 bp, unidentified
  misc_feature 1..344: note = Description of Artificial Sequence: Synthetic polynucleotide
  source 1..344: mol_type = other DNA; note = Simian virus 40
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagct

SEQ ID NO: 40 - DNA, 1229 bp, synthetic construct
  misc_feature 1..1229: note = Description of Artificial Sequence: Synthetic polynucleotide
  source 1..1229: mol_type = other DNA
gcgccgggttttggcgcctcccgcgggcgcccccctcctcacggcgagcgctgccacgtcagacgaagggcgcaggagcgttcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagttgcgggctgctgggctggccggggctttcgtggccgccgggccgctcggtgggacggaagcgtgtggagagaccgccaagggctgtagtctgggtccgcgagcaaggttgccctgaactgggggttggggggagcgcacaaaatggcggctgttcccgagtcttgaatggaagacgcttgtaaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcgggtttgtcgtctggttgcgggggcggcagttatgcggtgccgttgggcagtgcacccgtacctttgggagcgcgcgcctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaagcttctgcaggtcgactctagaaaattgtccgctaaattctggccgtttttggcttttttgttagac

SEQ ID NO: 41 - DNA, 1179 bp, synthetic construct
  misc_feature 1..1179: note = Description of Artificial Sequence: Synthetic polynucleotide
  source 1..1179: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 42 - DNA, 1718 bp, synthetic construct
  misc_feature 1..1718: note = Description of Artificial Sequence: Synthetic polynucleotide
  source 1..1718: mol_type = other DNA
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattc

SEQ ID NO: 43 - DNA, 212 bp, synthetic construct
  misc_feature 1..212: note = Description of Artificial Sequence: Synthetic polynucleotide
  source 1..212: mol_type = other DNA
gggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 44 - DNA, 2379 bp, synthetic construct
  misc_feature 1..2379: note = Description of Artificial Sequence: Synthetic polynucleotide
  source 1..2379: mol_type = other DNA
ccactagttccatgtccttatatggactcatctttgcctattgcgacacacactcaatgaacacctactacgcgctgcaaagagccccgcaggcctgaggtgcccccacctcaccactcttcctatttttgtgtaaaaatccagcttcttgtcaccacctccaaggagggggaggaggaggaaggcaggttcctctaggctgagccgaatgcccctctgtggtcccacgccactgatcgctgcatgcccaccacctgggtacacacagtctgtgattcccggagcagaacggaccctgcccacccggtcttgtgtgctactcagtggacagacccaaggcaagaaagggtgacaaggacagggtcttcccaggctggctttgagttcctagcaccgccccgcccccaatcctctgtggcacatggagtcttggtccccagagtcccccagcggcctccagatggtctgggagggcagttcagctgtggctgcgcatagcagacatacaacggacggtgggcccagacccaggctgtgtagacccagcccccccgccccgcagtgcctaggtcacccactaacgccccaggcctggtcttggctgggcgtgactgttaccctcaaaagcaggcagctccagggtaaaaggtgccctgccctgtagagcccaccttccttcccagggctgcggctgggtaggtttgtagccttcatcacgggccacctccagccactggaccgctggcccctgccctgtcctggggagtgtggtcctgcgacttctaagtggccgcaagccacctgactcccccaacaccacactctacctctcaagcccaggtctctccctagtgacccacccagcacatttagctagctgagccccacagccagaggtcctcaggccctgctttcagggcagttgctctgaagtcggcaagggggagtgactgcctggccactccatgccctccaagagctccttctgcaggagcgtacagaacccagggccctggcacccgtgcagaccctggcccaccccacctgggcgctcagtgcccaagagatgtccacacctaggatgtcccgcggtgggtggggggcccgagagacgggcaggccgggggcaggcctggccatgcggggccgaaccgggcactgcccagcgtggggcgcgggggccacggcgcgcgcccccagcccccgggcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagaccgcgtccgccccgcgagcacagagcctcgcctttgccgatccgccgcccgtccacacccgccgccaggtaagcccggccagccgaccggggcaggcggctcacggcccggccgcaggcggccgcggccccttcgcccgtgcagagccgccgtctgggccgcagcggggggcgcatggggggggaaccggaccgccgtggggggcgcgggagaagcccctgggcctccggagatgggggacaccccacgccagttcggaggcgcgaggccgcgctcgggaggcgcgctccgggggtgccgctctcggggcgggggcaaccggcggggtctttgtctgagccgggctcttgccaatggggatcgcagggtgggcgcggcggagcccccgccaggcccggtgggggctggggcgccattgcgcgtgcgcgctggtcctttgggcgctaactgcgtgcgcgctgggaattggcgctaattgcgcgtgcgcgctgggactcaaggcgctaactgcgcgtgcgttctggggcccggggtgccgcggcctgggctggggcgaaggcgggctcggccggaaggggtggggtcgccgcggctcccgggcgcttgcgcgcacttcctgcccgagccgctggccgcccgagggtgtggccgctgcgtgcgcgcgcgccgacccggcgctgtttgaaccgggcggaggcggggctggcgcccggttgggagggggttggggcctggcttcctgccgcgcgccgcggggacgcctccgaccagtgtttgccttttatggtaataacgcggccggcccggcttcctttgtccccaatctgggcgcgcgccggcgccccctggcggcctaaggactcggcgcgccggaagtggccagggcgggggcgacctcggctcacagcgcgcccggctat

SEQ ID NO: 45 - DNA, 529 bp, synthetic construct
  misc_feature 1..529: note = Description of Artificial Sequence: Synthetic polynucleotide
  source 1..529: mol_type = other DNA
gttgatttccttcatccctggcacacgtccaggcagtgtcgaatccatctctgctacaggggaaaacaaataacatttgagtccagtggagaccgggagcagaagtaaagggaagtgataacccccagagcccggaagcctctggaggctgagacctcgccccccttgcgtgatagggcctacggagccacatgaccaaggcactgtcgcctccgcacgtgtgagagtgcagggccccaagatggctgccaggcctcgaggcctgactcttctatgtcacttccgtaccggcgagaaaggcgggccctccagccaatgaggctgcggggcgggccttcaccttgataggcactcgagttatccaatggtgcctgcgggccggagcgactaggaactaacgtcatgccgagttgctgagcgccggcaggcggggccggggcggccaaaccaatgcgatggccggggcggagtcgggcgctctataagttgtcgataggcgggcactccgccctagtttctaaggaccatg

SEQ ID NO: 46 - DNA, 794 bp, synthetic construct
  misc_feature 1..794: note = Description of Artificial Sequence: Synthetic polynucleotide
  source 1..794: mol_type = other DNA
agttccccaactttcccgcctctcagcctttgaaagaaagaaaggggagggggcaggccgcgtgcagtcgcgagcggtgctgggctccggctccaattccccatctcagtcgctcccaaagtccttctgtttcatccaagcgtgtaagggtccccgtccttgactccctagtgtcctgctgcccacagtccagtcctgggaaccagcaccgatcacctcccatcgggccaatctcagtcccttcccccctacgtcggggcccacacgctcggtgcgtgcccagttgaaccaggcggctgcggaaaaaaaaaagcggggagaaagtagggcccggctactagcggttttacgggcgcacgtagctcaggcctcaagaccttgggctgggactggctgagcctggcgggaggcggggtccgagtcaccgcctgccgccgcgcccccggtttctataaattgagcccgcagcctcccgcttcgctctctgctcctcctgttcgacagtcagccgcatcttcttttgcgtcgccaggtgaagacgggcggagagaaacccgggaggctagggacggcctgaaggcggcaggggcgggcgcaggccggatgtgttcgcgccgctgcggggtgggcccgggcggcctccgcattgcaggggcgggcggaggacgtgatgcggcgcgggctgggcatggaggcctggtgggggaggggaggggaggcgtgggtgtcggccggggccactaggcgctcactgttctctccctccgcgcagccgagccacatcgctgagacac

SEQ ID NO: 47 - DNA, 532 bp, synthetic construct
  misc_feature 1..532: note = Description of Artificial Sequence: Synthetic polynucleotide
  source 1..532: mol_type = other DNA
agtgcggttaccagcggaaatgcctcggggtcagaagtcgcaggagagatagacagctgctgaaccaatgggaccagcggatggggcggatgttatctaccattggtgaacgttagaaacgaatagcagccaatgaatcagctgggggggcggagcagtgacgtttattgcggagggggccgcttcgaatcggcggcggccagcttggtggcctgggccaatgaacggcctccaacgagcagggccttcaccaatcggcggcctccacgacggggctgggggagggtatataagccgagtaggcgacggtgaggtcgacgccggccaagacagcacagacagattgacctattggggtgtttcgcgagtgtgagagggaagcgccgcggcctgtatttctagacctgcccttcgcctggttcgtggcgccttgtgaccccgggcccctgccgcctgcaagtcggaaattgcgctgtgctcctgtgctacggcctgtggctggactgcctgctgctgcccaactggctggcac

SEQ ID NO: 48 - DNA, 701 bp, synthetic construct
  misc_feature 1..701: note = Description of Artificial Sequence: Synthetic polynucleotide
  source 1..701: mol_type = other DNA
tagtttcatcaccaccgccacccccccgcccccccgccatctgaaagggttctaggggatttgcaacctctctcgtgtgtttcttctttccgagaagcgccgccacacgagaaagctggccgcgaaagtcgtgctggaatcacttccaacgaaaccccaggcatagatgggaaagggtgaagaacacgttgccatggctaccgtttccccggtcacggaataaacgctctctaggatccggaagtagttccgccgcgacctctctaaaaggatggatgtgttctctgcttacattcattggacgttttcccttagaggccaaggccgcccaggcaaaggggcggtcccacgcgtgaggggcccgcggagccatttgattggagaaaagctgcaaaccctgaccaatcggaaggagccacgcttcgggcatcggtcaccgcacctggacagctccgattggtggacttccgccccccctcacgaatcctcattgggtgccgtgggtgcgtggtgcggcgcgattggtgggttcatgtttcccgtcccccgcccgcgagaagtgggggtgaaaagcggcccgacctgcttggggtgtagtgggcggaccgcgcggctggaggtgtgaggatccgaacccaggggtggggggtggaggcggctcctgcgatcgaaggggacttgagactcaccggccgcacgtc

SEQ ID NO: 49 - DNA, 465 bp, synthetic construct
  misc_feature 1..465: note = Description of Artificial Sequence: Synthetic polynucleotide
  source 1..465: mol_type = other DNA
gggccgcccactcccccttcctctcagggtccctgtcccctccagtgaatcccagaagactctggagagttctgagcagggggcggcactctggcctctgattggtccaaggaaggctggggggcaggacgggaggcgaaaaccctggaatattcccgacctggcagcctcatcgagctcggtgattggctcagaagggaaaaggcgggtctccgtgacgacttataaaagcccaggggcaagcggtccggataacggctagcctgaggagctgctgcgacagtccactacctttttcgagagtgactcccgttgtcccaaggcttcccagagcgaacctgtgcggctgcaggcaccggcgcgtcgagtttccggcgtccggaaggaccgagctcttctcgcggatccagtgttccgtttccagcccccaatctcagagcggagccgacagagagcagggaaccc

SEQ ID NO: 50 - DNA, 538 bp, synthetic construct
  misc_feature 1..538: note = Description of Artificial Sequence: Synthetic polynucleotide
  source 1..538: mol_type = other DNA
gccccacccccgtccgcgttacaaccgggaggcccgctgggtcctgcaccgtcaccctcctccctgtgaccgcccacctgatacccaaacaactttctcgcccctccagtccccagctcgccgagcgcttgcggggagccacccagcctcagtttccccagccccgggcggggcgaggggcgatgacgtcatgccggcgcgcggcattgtggggcggggcgaggcggggcgccggggggagcaacactgagacgccattttcggcggcgggagcggcgcaggcggccgagcgggactggctgggtcggctgggctgctggtgcgaggagccgcggggctgtgctcggcggccaaggggacagcgcgtgggtggccgaggatgctgcggggcggtagctccggcgcccctcgctggtgactgctgcgccgtgcctcacacagccgaggcgggctcggcgcacagtcgctgctccgcgctcgcgcccggcggcgctccaggtgctgacagcgcgagagagcgcggcctcaggagcaacac

SEQ ID NO: 51 - DNA, 1091 bp, synthetic construct
  misc_feature 1..1091: note = Description of Artificial Sequence: Synthetic polynucleotide
  source 1..1091: mol_type = other DNA
ttccagagctttcgaggaaggtttcttcaactcaaattcatccgcctgataattttcttatattttcctaaagaaggaagagaagcgcatagaggagaagggaaataattttttaggagcctttcttacggctatgaggaatttggggctcagttgaaaagcctaaactgcctctcgggaggttgggcgcggcgaactactttcagcggcgcacggagacggcgtctacgtgaggggtgataagtgacgcaacactcgttgcataaatttgcgctccgccagcccggagcatttaggggcggttggctttgttgggtgagcttgtttgtgtccctgtgggtggacgtggttggtgattggcaggatcctggtatccgctaacaggtactggcccacagccgtaaagacctgcgggggcgtgagaggggggaatgggtgaggtcaagctggaggcttcttggggttgggtgggccgctgaggggaggggagggcgaggtgacgcgacacccggcctttctgggagagtgggccttgttgacctaaggggggcgagggcagttggcacgcgcacgcgccgacagaaactaacagacattaaccaacagcgattccgtcgcgtttacttgggaggaaggcggaaaagaggtagtttgtgtggcttctggaaaccctaaatttggaatcccagtatgagaatggtgtcccttcttgtgtttcaatgggatttttacttcgcgagtcttgtgggtttggttttgttttcagtttgcctaacaccgtgcttaggtttgaggcagattggagttcggtcgggggagtttgaatatccggaacagttagtggggaaagctgtggacgcttggtaagagagcgctctggattttccgctgttgacgttgaaaccttgaatgacgaatttcgtattaagtgacttagccttgtaaaattgaggggaggcttgcggaatattaacgtatttaaggcattttgaaggaatagttgctaattttgaagaatattaggtgtaaaagcaagaaatacaatgatcctgaggtgacacgcttatgttttacttttaaactaggtcacc

SEQ ID NO: 52 - protein, 121 aa, synthetic construct
  REGION 1..121: note = Description of Artificial Sequence: Synthetic polypeptide
GSQVQLVESGGGLVQAGGSLRLSCTASGRTGTIYSMAWFRQAPGKEREFLATVGWSSGITYYMDSVKGRFTISRDKGKNTVYLQMDSLKPEDTAVYYCTATRAYSVGYDYWGQGTQVTVSS

SEQ ID NO: 53 - protein, 119 aa, synthetic construct
  REGION 1..119: note = Description of Artificial Sequence: Synthetic polypeptide
EVQLQASGGGFVQPGGSLRLSCAASGSTSRQYDMGWFRQAPGKEREFVSAISSNQDQPPYYADSVKGRFTISRDNSKNTVYLQMNSLRAEDTATYYCAFKQHHANGAYWGQGTQVTVSS

SEQ ID NO: 54 - protein, 119 aa, synthetic construct
  REGION 1..119: note = Description of Artificial Sequence: Synthetic polypeptide
EVQLQASGGGFVQPGGSLRLSCAASGRFSWGEEMGWFRQAPGKEREFVSAISWAATPWQYYADSVKGRFTISRDNSKNTVYLQMNSLRAEDTATYYCADEWHIGHVSYWGQGTQVTVSS

SEQ ID NO: 55 - protein, 119 aa, synthetic construct
  REGION 1..119: note = Description of Artificial Sequence: Synthetic polypeptide
EVQLQASGGGFVQPGGSLRLSCAASGTTSDNDTMGWFRQAPGKEREFVSAISWNGGRDEYYADSVKGRFTISRDNSKNTVYLQMNSLRAEDTATYYCAYQDNRSWQEYWGQGTQVTVSS

SEQ ID NO: 56 - protein, 119 aa, synthetic construct
  REGION 1..119: note = Description of Artificial Sequence: Synthetic polypeptide
EVQLQASGGGFVQPGGSLRLSCAASGGYSRADDMGWFRQAPGKEREFVSAISFGETDSFYYADSVKGRFTISRDNSKNTVYLQMNSLRAEDTATYYCAYHNYTNMFEYWGQGTQVTVSS

SEQ ID NO: 57 - protein, 119 aa, synthetic construct
  REGION 1..119: note = Description of Artificial Sequence: Synthetic polypeptide
EVQLQASGGGFVQPGGSLRLSCAASGTTYGQTNMGWFRQAPGKEREFVSAISGLQGRDLYYADSVKGRFTISRDNSKNTVYLQMNSLRAEDTATYYCAFHDFLRMWEYWGQGTQVTVSS

SEQ ID NO: 58 - protein, 116 aa, synthetic construct
  REGION 1..116: note = Description of Artificial Sequence: Synthetic polypeptide
QMQLLESGPGLVKPSETLSLTCTVSGGSIWGWIRQPPGKGLEWIGSIYSSGSTYYNPSLKSRVTTSVDTSKNQFSLRLSSVTAADTAVYYCVAWFGDLLSLKGVELWGQGTLVTVS

SEQ ID NO: 59 - protein, 111 aa, synthetic construct
  REGION 1..111: note = Description of Artificial Sequence: Synthetic polypeptide
QSELTQPPSASGTPGQRVTISCSGSSSNIGSNYVYWYQQLPGTAPKLLIYRNNQRPSGVPDRFSGSKSGTSASLAISGLRSEDEADYYCAAWDDSLSAWVFGGGTQLDILG

SEQ ID NO: 60 - protein, 107 aa, synthetic construct
  REGION 1..107: note = Description of Artificial Sequence: Synthetic polypeptide
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 61 - protein, 93 aa, synthetic construct [sequence omitted]

SEQ ID NO: 62 - DNA, 18 bp, synthetic construct
  misc_feature 1..18: note = Description of Artificial Sequence: Synthetic oligonucleotide
  source 1..18: mol_type = other DNA
ggcgtagccgatgtcgcg

SEQ ID NO: 63 - DNA, 172 bp, synthetic construct
  misc_feature 1..172: note = Description of Artificial Sequence: Synthetic polynucleotide
  source 1..172: mol_type = other DNA
cgggtttcgtaacaatcgcatgaggattcgcaacgccttcggcgtagccgatgtcgcgctcccgtctcagtaaaggtcggcgtagccgatgtcgcgcaatcggactgccttcgtacggcgtagccgatgtcgcgcgtatcagtcgcctcggaacggcgtagccgatgtcgcg

SEQ ID NO: 64 - DNA, 25 bp, synthetic construct
  misc_feature 1..25: note = Description of Artificial Sequence: Synthetic oligonucleotide
  source 1..25: mol_type = other DNA
tctagagggtatataatgggggcca

SEQ ID NO: 65 - DNA, 243 bp, synthetic construct
  misc_feature 1..243: note = Description of Artificial Sequence: Synthetic polynucleotide
  source 1..243: mol_type = other DNA
cgggtttcgtaacaatcgcatgaggattcgcaacgccttcggcgtagccgatgtcgcgctcccgtctcagtaaaggtcggcgtagccgatgtcgcgcaatcggactgccttcgtacggcgtagccgatgtcgcgcgtatcagtcgcctcggaacggcgtagccgatgtcgcgcattcgtaagaggctcactctcccttacacggagtggataactagttctagagggtatataatgggggcca

SEQ ID NO: 66 - DNA, 672 bp, synthetic construct
  misc_feature 1..672: note = Description of Artificial Sequence: Synthetic polynucleotide
  source 1..672: mol_type = other DNA
tctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagctgacgtcctttccatggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctgtttcgcctcggcgtccggtccgtgttgcttggtcgtcacctgtgcagaattgcgaaccatggattcca

SEQ ID NO: 67 - DNA, 672 bp, synthetic construct
  misc_feature 1..672: note = Description of Artificial Sequence: Synthetic polynucleotide
  source 1..672: mol_type = other DNA
tctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctgtttcgcctcggcgtccggtccgtgttgcttggtcgtcacctgtgcagaattgcgaaccatggattcca

SEQ ID NO: 68 - protein, 299 aa, synthetic construct
  REGION 1..299: note = Description of Artificial Sequence: Synthetic polypeptide
GIRKFKKFNKVRVVRALDAVALPQPLGVPNESQALSQRFTFSPGQDIQLIPPLINLLMSIEPDVIYAGHDNTKPDTSSSLLTSLNQLGERQLLSVVKWSKSLPGFRNLHIDDQITLIQYSWMSLMVFGLGWRSYKHVSGQMLYFAPDLILNEQRMKESSFYSLCLTMWQIPQEFVKLQVSQEEFLCMKVLLLLNTIPLEGLRSQTQFEEMRSSYIRELIKAIGLRQKGVVSSSQRFYQLTKLLDNLHDLVKQLHLYCLNTFIQSRALSVEFPEMMSEVIAAQLPKILAGMVKPLLFHKK

SEQ ID NO: 69 - DNA, 780 bp, synthetic construct
  misc_feature 1..780: note = Description of Artificial Sequence: Synthetic polynucleotide
  source 1..780: mol_type = other DNA
cagtacctgccagatacagacgatcgtcaccggattgaggagaaacgtaaaaggacatatgagaccttcaagagcatcatgaagaagagtcctttcagcggacccaccgacccccggcctccacctcgacgcattgctgtgccttcccgcagctcagcttctgtccccaagccagcaccccagccctatccctttacgtcatccctgagcaccatcaactatgatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctg

SEQ ID NO: 70 - protein, 260 aa, synthetic construct
  REGION 1..260: note = Description of Artificial Sequence: Synthetic polypeptide
QYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALL

SEQ ID NO: 71 - DNA, 123 bp, synthetic construct
  misc_feature 1..123: note = Description of Artificial Sequence: Synthetic polynucleotide
  source 1..123: mol_type = other DNA
cagtgtaccaactacgccctgctgaaactggccggcgacgtggaatctaatcctggacctggatctggcgagggacgcgggagtctactgacgtgtggagacgtggaggaaaaccctggacct

SEQ ID NO: 72 - protein, 41 aa, synthetic construct
  REGION 1..41: note = Description of Artificial Sequence: Synthetic polypeptide
QCTNYALLKLAGDVESNPGPGSGEGRGSLLTCGDVEENPGP

SEQ ID NO: 73 - DNA, 699 bp, synthetic construct
  misc_feature 1..699: note = Description of Artificial Sequence: Synthetic polynucleotide
  source 1..699: mol_type = other DNA
atgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaat

SEQ ID NO: 74 - protein, 233 aa, synthetic construct
  REGION 1..233: note = Description of Artificial Sequence: Synthetic polypeptide
MSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 75 - DNA, 134 bp, synthetic construct
  misc_feature 1..134: note = Description of Artificial Sequence: Synthetic polynucleotide
  source 1..134: mol_type = other DNA
cgggtttcgtaacaatcgcatgaggattcgcaacgccttcggcgtagccgatgtcgcgctcccgtctcagtaaaggtcggcgtagccgatgtcgcgcaatcggactgccttcgtacggcgtagccgatgtcgcg

SEQ ID NO: 76 - protein, 4 aa, synthetic construct
  REGION 1..4: note = Description of Artificial Sequence: Synthetic peptide
WRPW